(19) United States
(12) Patent Application Publication
(10) Pub. No.: US 2006/0121466 A1
(43) Pub. Date: Jun. 8, 2006

(54) Hammerhead ribozymes

(75) Inventors: Anastasia Khvorova, Boulder, CO (US); Sumedha Jayasena, Thousand Oaks, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: AMGEN INC., Thousand Oaks, CA (US)

(21) Appl. No.: 10/517,638

(22) PCT Filed: Jun. 12, 2003
(86) PCT No.: PCT/US03/18499

§ 371 Date: Dec. 13, 2004

Related U.S. Application Data
(60) Provisional application No. 60/389,147, filed on Jun. 13, 2002.

(51) Int. Cl. C12Q 1/68; C07H 21/02; C12P 21/06

(52) U.S. Cl. 435/6; 435/69.1; 435/320.1; 435/325; 536/23.1

(57) ABSTRACT

The present invention relates to non-natural cis- and trans-cleaving hammerhead ribozymes and methods for selecting non-natural ribozymes. The present invention also relates to methods of cleaving a target RNA, methods for reducing the level of a target RNA in a cell, and methods of reducing the level of a protein in a cell. The present invention also provides pharmaceutical compositions comprising the non-natural ribozymes of the invention.

### FIELD OF THE INVENTION

The present invention relates to hammerhead ribozymes. The present invention also relates to methods of selecting hammerhead ribozymes that cleave in the presence of low Mg²⁺. The present invention also relates to methods of cleaving RNA in vitro and in vivo.

### BACKGROUND OF THE INVENTION

Hammerhead ribozymes have been identified as motifs within a number of plant viroids. A viroid is circular single-stranded RNA of about 240 to 400 nucleotides that typically does not encode any proteins. Many viroids have been found to contain hammerhead and/or hairpin ribozymes, which are proposed to cleave multimeric RNA strands into monomeric viroid units following rolling-circle replication.

Hammerhead ribozymes typically comprise a highly conserved 13 nucleotide core, which is located at the junction of three helices, or stems. Naturally-occurring ribozymes, such as those found in plant viroids, are typically cis-cleaving ribozymes, which means that they are comprised of a single contiguous RNA molecule that cleaves itself.

In designing certain synthetic ribozymes that target desired sequences or have altered stem and loop structures, researchers have found that efficient in vitro cleavage requires very high concentrations of magnesium (Mg²⁺). In certain embodiments, it may be useful to have ribozymes that cleave at physiological Mg²⁺ concentrations. In certain embodiments, it may be useful to have ribozymes that cleave at low Mg²⁺ concentrations.

### SUMMARY OF THE INVENTION

In certain embodiments, the invention provides a non-natural cis-cleaving hammerhead ribozyme comprises a core, a stem I, a stem II, a stem III, a loop I, and a loop II. In certain embodiments, loop I and loop II are derived from loop I and loop II of a first hammerhead ribozyme selected from cherry small circular RNA+ (Scc+), cherry small circular RNA− (Scc−), Lucerne transient streak virusoid+ (sLTSV+), Lucerne transient streak virusoid− (sLTSV−), Tobacco ringspot virus satellite RNA+ (sTRSV+), Arabis mosaic virus (sArMV), Chicory yellow mottle virus satellite RNA (sCYMV), Barley yellow dwarf virus satellite RNA− (sBYDV−), Barley yellow dwarf virus satellite RNA+ (sBYDV+), Peach latent mosaic virus RNA+ (PLMVd+), Peach latent mosaic virus RNA− (PLMVd−), Chrysanthemum chlorotic mottle viroid+ (CChMVd+), Chrysanthemum chlorotic mottle viroid− (CChMVd−), Subterraneum clover mottle virusoid (vSCMoV), and velvet tobacco mottle virusoid (vVTMoV). In certain embodiments, at least one of stem I, stem II, and stem III is derived from a second hammerhead ribozyme that is not the same as the first hammerhead ribozyme.

In certain embodiments, a non-natural cis-cleaving hammerhead ribozyme comprises a core, a stem I, a stem II, a stem III, a bulge within stem I, and a loop II. In certain embodiments, loop II and the bulge within stem I are derived from loop II and a bulge within stem I of a first hammerhead ribozyme selected from Notophthalmus viridescens satellite RNA (newt), Ambystoma talpoideum (Am. ta.), Amphiuma tridactylum (Am. tr.), Schistosoma mansoni hammerhead ribozyme (Schistozyme), D. baccettii cricket hammerhead ribozyme (cricketzyme A), D. schiavazzii cricket hammerhead ribozyme (cricketzyme B), and Avocado sunblotch viroid+ (ASBV+). In certain embodiments, at least one of stem I, stem II, and stem III is derived from a second hammerhead ribozyme that is not the same as the first hammerhead ribozyme.

In certain embodiments, a non-natural cis-cleaving hammerhead ribozyme comprises a core, a stem I, a stem II, a stem III, loop I, and a bulge within stem II. In certain embodiments, loop I and the bulge within stem II are derived from a first ribozyme selected from Chrysanthemum chlorotic mottle viroid− (CChMVd−) and Barley yellow dwarf virus satellite RNA (sBYDV+). In certain embodiments, at least one of stem I, stem II, and stem III is derived from a second hammerhead ribozyme that is not the same as the first hammerhead ribozyme.

In certain embodiments, a non-natural cis-cleaving hammerhead ribozyme comprises a core, a stem I, a stem II, a stem II, a bulge within stem I, and a bulge within stem II. In certain embodiments, the bulge within stem I and the bulge within stem II are derived from a first ribozyme selected from Avocado sunblotch viroid− (ASBV−) and Carnation small viroid-like RNA+ (CarSV+). In certain embodiments, at least one of stem I, stem II, and stem III is derived from a second hammerhead ribozyme that is not the same as the first hammerhead ribozyme.

In certain embodiments, the invention provides a non-natural cis-cleaving hammerhead ribozyme comprising structure III, shown in FIG. 41A(3). In certain embodiments, J, K, and N are each independently selected from A, C, G, and U; Y is selected from C and U; H is selected from A, C, and U; a is a sequence of 4-20 nucleotides, wherein each nucleotide is independently selected from A, C, G, and U; b, c, d, e, f, and g are each a sequence of 2-20 nucleotides, wherein each nucleotide is independently selected from A, C, G, and U; h is a sequence of 1-20 nucleotides, wherein each nucleotide is independently selected from A, C, G, and U; a, b, JKCGUACG, and c together are stem I; d and e together are stem II; and f and g together are at least a portion of stem III. In certain embodiments, the invention provides a non-natural hammerhead ribozyme, wherein at least a portion of f and g together comprises an aptamer capable of binding a small molecule.

In certain embodiments, the invention provides a non-natural cis-cleaving hammerhead ribozyme comprising structure IV, shown in FIG. 41A(4). In certain embodiments, N is selected from A, C, G, and U; Y is selected from C and U; X is selected from G and C; Z is selected from G and A; H is selected from A, C, and U; a is a sequence of 4-20 nucleotides, wherein each nucleotide is independently selected from A, C, G, and U; b, c, d, e, f, and g are each a sequence of 2-20 nucleotides, wherein each nucleotide is independently selected from A, C, G, and U; h is a sequence of 1-20 nucleotides, wherein each nucleotide is independently selected from A, C, G, and U; a, b, GM, and c together are stem I; d and e together are stem II; and f and g together are stem III. In certain embodiments, the invention provides a non-natural hammerhead ribozyme, wherein at least a portion of f and g together comprises an aptamer capable of binding a small molecule.

In certain embodiments, the invention provides a non-natural cis-cleaving hammerhead ribozyme comprising structure VI, shown in FIG. 41B(6). In certain embodiments, J, K, and N are each independently selected from A, C, G, and U; Y is selected from C and U; H is selected from A, C, and U; a is a sequence of 4-20 nucleotides, wherein each nucleotide is independently selected from A, C, G, and U; b, c, d, e, f, and g are each a sequence of 2-20 nucleotides, wherein each nucleotide is independently selected from A, C, G, and U; m is a sequence of 1-20 nucleotides, wherein each nucleotide is independently selected from A, C, G, and U; a, b, JKCGUACG, and c together are stem I; d and e together are stem II; and f and g together are at least a portion of stem III. In certain embodiments, the invention provides a non-natural hammerhead ribozyme, wherein at least a portion of f and g together comprises an aptamer capable of binding a small molecule.

In certain embodiments, the invention provides a non-natural cis-cleaving hammerhead ribozyme comprising structure VII, shown in FIG. 41B(7). In certain embodiments, N is selected from A, C, G, and U; Y is selected from C and U; X is selected from G and C; Z is selected from G and A; H is selected from A, C, and U; a is a sequence of 4-20 nucleotides, wherein each nucleotide is independently selected from A, C, G, and U; b, c, d, e, f, and g are each a sequence of 2-20 nucleotides, wherein each nucleotide is independently selected from A, C, G, and U; m is a sequence of 1-20 nucleotides, wherein each nucleotide is independently selected from A, C, G, and U; a, b, GM, and c together are stem I; d and e together are stem II; and f and g together are stem III. In certain embodiments, the invention provides a non-natural hammerhead ribozyme, wherein at least a portion of f and g together comprises an aptamer capable of binding a small molecule.

In certain embodiments, the invention provides a non-natural cis-cleaving hammerhead ribozyme that cis-cleaves at an initial rate of at least 0.5 min⁻¹ in a buffer comprising 50 mM Tris (pH 7.0) and 1 mM Mg²⁺ at 37° C. In certain embodiments, the invention provides a non-natural cis-cleaving hammerhead ribozyme that cis-cleaves at an initial rate of at least 0.5 min⁻¹ in a buffer comprising 50 mM Tris (pH 7.0) and 0.5 mM Mg²⁺ at 37° C. In certain embodiments, the invention provides a non-natural cis-cleaving hammerhead ribozyme that cis-cleaves at an initial rate of at least 0.5 min⁻¹ in a buffer comprising 50 mM Tris (pH 7.0) and 0.1 mM Mg²⁺ at 37° C.

In certain embodiments, the invention provides a polynucleotide comprising a first nucleic acid sequence, wherein the first nucleic acid sequence encodes a non-natural cis-cleaving hammerhead ribozyme. In certain embodiments, the polynucleotide further comprises a second nucleic acid sequence, wherein the second nucleic acid sequence encodes an RNA that is not a non-natural hammerhead ribozyme. In certain embodiments, the first nucleic acid sequence is inserted in frame into the second nucleic acid sequence. In certain embodiments, the second nucleic acid sequence comprises a non-coding region, wherein the non-coding region is selected from a 3′-untranslated region (3′-UTR), a 5′-untranslated region (5′-UTR), and an intron. In certain embodiments, the first nucleic acid sequence is inserted into the non-coding region.

In certain embodiments, the invention provides a vector comprising that polynucleotide. In certain embodiments, the invention provides a host cell comprising the polynucleotide.

In certain embodiments, the invention provides a non-natural trans-cleaving hammerhead ribozyme comprising a core, a stem I, a stem II, a stem III, a bulge within stem I, and a loop II. In certain embodiments, loop II and the bulge within stem I are derived from loop II and a bulge within stem I of a first hammerhead ribozyme selected from Notophthalmus viridescens satellite RNA (newt), Ambystoma talpoideum (Am. ta.), Amphiuma tridactylum (Am. tr.), Schistosoma mansoni hammerhead ribozyme (Schistozyme), D. baccettii cricket hammerhead ribozyme (cricketzyme A), D. schiavazzii cricket hammerhead ribozyme (cricketzyme B), and Avocado sunblotch viroid+ (ASBV+). In certain embodiments, at least one of stem II, stem II, and a portion of stem I is derived from a second hammerhead ribozyme that is not the same as the first hammerhead ribozyme.

In certain embodiments, the invention provides a non-natural trans-cleaving hammerhead ribozyme comprising a core, a stem I, a stem II, a stem II, a bulge within stem I, and a bulge within stem II. In certain embodiments, the bulge within stem I and the bulge within stem II are derived from a first ribozyme selected from Avocado sunblotch viroid− (ASBV−) and Carnation small viroid-like RNA+ (CarSV+). In certain embodiments, at least one of stem II, a portion of stem I, and a portion of stem II is derived from a second hammerhead ribozyme that is not the same as the first hammerhead ribozyme.

In certain embodiments, a non-natural trans-cleaving hammerhead ribozyme comprising strand A of structure I, shown in FIG. 41A(1), is provided. In certain embodiments, the non-natural trans-cleaving hammerhead ribozyme cleaves a target RNA sequence comprising strand B of structure 1. In certain embodiments, J, K, and N are each independently selected from A, C, G, and U; Y is selected from C and U; H is selected from A, C, and U; a is a sequence of 4-20 nucleotides, wherein each nucleotide is independently selected from A, C, G, and U; b, c, d, e, f, and g are each a sequence of 2-20 nucleotides, wherein each nucleotide is independently selected from A, C, G, and U; a, b, JKCGUACG, and c together are stem I; d and e together are stem II; and f and g together are at least a portion of stem III.

In certain embodiments, a non-natural trans-cleaving hammerhead ribozyme comprising strand A of structure II, shown in FIG. 41A(2), is provided. In certain embodiments, N is selected from A, C, G, and U; Y is selected from C and U; X is selected from G and C; Z is selected from G and A; H is selected from A, C, and U; a is a sequence of 4-20 nucleotides, wherein each nucleotide is independently selected from A, C, G, and U; b, c, d, e, f, and g are each a sequence of 2-20 nucleotides, wherein each nucleotide is independently selected from A, C, G, and U; a, b, GAA, and c together are stem I; d and e together are stem II; and f and g together are stem III.

In-certain embodiments, the invention provides a non-natural trans-cleaving hammerhead ribozyme that cleaves the target RNA sequence at an initial rate of at least 0.5 min⁻¹ in a buffer comprising 50 mM Tris (pH 7.0) and 1 mM Mg²⁺ at 37° C. In certain embodiments, the non-natural trans-cleaving hammerhead ribozyme cleaves the target RNA sequence at an initial rate of at least 0.5 min¹ in a buffer comprising 50 mM Tris (pH 7.0) and 0.5 mM Mg²⁺ at 37° C. In certain embodiments, the non-natural trans-cleaving hammerhead ribozyme cleaves the target RNA sequence at an initial rate of at least 0.5 min⁻¹ in a buffer comprising 50 mM Tris (pH 7.0) and 0.1 mM Mg²⁺ at 37° C.

In certain embodiments, the invention provides a polynucleotide comprising a nucleic acid encoding a non-natural trans-cleaving hammerhead ribozyme. In certain embodiments, the invention provides a vector comprising that polynucleotide. In certain embodiments, the invention provides a host cell comprising the polynucleotide.

In certain embodiments, the invention provides a method of cleaving a target RNA sequence in a cell comprising introducing into a cell a polynucleotide comprising a nucleic acid encoding a non-natural trans-cleaving hammerhead ribozyme. In certain embodiments, a method of cleaving a target RNA sequence in a cell comprises introducing into a cell a polynucleotide comprising a nucleic acid encoding a non-natural trans-cleaving hammerhead ribozyme.

In certain embodiments, the invention provides a pharmaceutical composition comprising a polynucleotide comprising a nucleic acid encoding a non-natural trans-cleaving hammerhead ribozyme in a pharmaceutically acceptable carrier. In certain embodiments, a method of cleaving a target RNA in a mammal comprising administering the pharmaceutical composition is provided. In certain embodiments, the invention provides a method of decreasing the level of a target RNA in a mammal comprising administering the pharmaceutical composition. In certain embodiments, the invention provides a method of decreasing the level of a target RNA in a mammal comprising administering the pharmaceutical composition. In certain embodiments, the invention provides a method of decreasing the expression of a protein encoded by a target RNA in a mammal comprising administering the pharmaceutical composition.

In certain embodiments, the invention provides a method of making a trans-cleaving hammerhead ribozyme, comprising forming a library of hammerhead ribozyme molecules having structure V, shown in FIG. 41A(5). In certain embodiments, N is selected from A, C, G, and U; Y is selected from C and U; H is selected from A, C, and U; a is a sequence of 4-20 nucleotides, wherein each nucleotide is independently selected from A, C, G, and U; b, c, d, e, f, and g are each a sequence of 2-20 nucleotides, wherein each nucleotide is independently selected from A, C, G, and U; h is a sequence of 1-20 nucleotides, wherein each nucleotide is independently selected from A, C, G, and U; j is a bulge of 1-20 nucleotides, wherein each nucleotide is individually selected from A, C, G, and U; k is a loop of 1-20 nucleotides, wherein each nucleotide is individually selected from A, C, G, and U; a, b, j, and c together are stem I; d and e together are stem II; and f and g together are stem III. In certain embodiments, each member of the library comprises the same N, Y, H, a, b, c, d, e, f, g, and h; and each member of the library does not comprise the same j and k. In certain embodiments, each member of the library comprises the same N, Y, H, a, b, c, d, e, f, g, h, and j; and each member of the library does not comprise the same k. In certain embodiments, each member of the library comprises the same N, Y, H, a, b, c, d, e, f, g, h, and k; and each member of the library does not comprise the same j.

In certain embodiments, the invention provides a method of making a trans-cleaving hammerhead ribozyme, comprising forming a library of hammerhead ribozyme molecules having structure VIII, shown in FIG. 41B(8). In certain embodiments, N is selected from A, C, G, and U; Y is selected from C and U; H is selected from A, C, and U; a is a sequence of 4-20 nucleotides, wherein each nucleotide is independently selected from A, C, G, and U; b, c, d, e, f, and g are each a sequence of 2-20 nucleotides, wherein each nucleotide is independently selected from A, C, G, and U; m is a sequence of 1-20 nucleotides, wherein each nucleotide is independently selected from A, G and U; j is a bulge of 1-20 nucleotides, wherein each nucleotide is individually selected from A, C, G, and U; k is a loop of 1-20 nucleotides, wherein each nucleotide is individually selected from A, C, G, and U; a, b, j, and c together are stem I; d and e together are stem II; and f and g together are stem III. In certain embodiments, each member of the library comprises the same N, Y, H, a, b, c, d, e, f, g, and m; and each member of the library does not comprise the same j and k. In certain embodiments, each member of the library comprises the same N, Y, H, a, b, c, d, e, f, g, m, and j; and each member of the library does not comprise the same k. In certain embodiments, each member of the library comprises the same N, Y, H, a, b, c, d, e, f, g, m, and k; and each member of the library does not comprise the same j.

In certain embodiments, the method further comprises incubating the library in the presence of less than 1 mM Mg²⁺. In certain embodiments, the method further comprises selecting a hammerhead ribozyme that cleaves at an initial rate of at least 0.5 min⁻¹. In certain embodiments, the selecting comprises denaturing polyacrylamide gel electrophoresis (denaturing PAGE). In certain embodiments, the method further comprises making a trans-cleaving hammerhead ribozyme comprising the nucleic acid sequence b, j, c, CYGANGA, d, k, e, GAAA, f. In certain embodiments, making the trans-cleaving hammerhead ribozyme comprises transcribing a DNA that encodes the sequence b, j, c, CYGANGA, d, k, e, GAAA, f. In certain embodiments, making the trans-cleaving hammerhead ribozyme comprises chemically synthesizing a nucleic acid comprising the sequence b, j, c, CYGANGA, d, k, e, GAAA, f.

### BRIEF DESCRIPTION OF THE FIGURES

FIG. 1 shows a schematic representation of certain embodiments of a stem with an asymmetric bulge (a), a stem with a symmetric bulge (b), and a stem with two asymmetric bulges (c).

FIG. 2 shows schematic representations of the cis-cleaving hammerhead ribozymes sTRSV and vLTSV−, and certain embodiments of chimeric ribozymes having the core and stems from sTRSV and loop I (sTRSV+vLT−1), loop II (sTRSV+vLT−2), or both loops (sTRSV+vLT−1 & 2) from vLTSV−. Loop sequences from sTRSV are indicated by an open circle (∘), and loop sequences from vLTSV are indicated by a closed circle (●).

FIG. 3 shows schematic representations of the cis-cleaving hammerhead ribozymes sTRSV and PLMVd, and certain embodiments of chimeric ribozymes having the core and stems from sTRSV and loop I (sTRSV+PL−1), loop II (sTRSV+PL−2), or both loops (sTRSV+PL−1 & 2) from PLMVd. Loop sequences from sTRSV are indicated by an open circle (∘), and loop sequences from PLMVd are indicated by a closed circle (●).

FIG. 4 shows the activity of certain wild-type and chimeric hammerhead ribozymes in 50 mM Tris-HCl (pH 7.0) and 1 mM MgCl₂. The activity is shown as the percentage of ribozyme that has cleaved as a function of time. FIG. 4A shows the activity of sTRSV, vLTSV−, and PLMVd. FIG. 4B shows the activity of sTRSV, vLTSV−, and chimeric ribozymes sTRSV+vLT1, sTRSV+vLT2, and sTRSV+vLT1 & vLT2. FIG. 4C shows the activity of sTRSV, PLMVd, and chimeric ribozymes sTRSV+PL1, sTRSV+PL2, and sTRSV +PL1 & PL2.

FIG. 5 shows the activity of certain wild-type and chimeric hammerhead ribozymes in phosphate-buffered saline (PBS) and 1 mM MgCl₂. FIG. 5A shows the activity of sTRSV, vLTSV−, and PLMVd. FIG. 5B shows the activity of sTRSV, vLTSV−, and chimeric ribozymes sTRSV+vLT1, sTRSV+vLT2, and sTRSV+vLT1 & vLT2. FIG. 5C shows the activity of sTRSV, PLMVd, and chimeric ribozymes sTRSV+PL1, sTRSV+PL2, and sTRSV+PL1 & PL2.

FIG. 6 shows the activity of certain wild-type and chimeric hammerhead ribozymes in 50 mM Tris-HCl (pH 7.0) and 0.1 mM MgCl₂. FIG. 6A shows the activity of sTRSV, vLTSV−, and PLMVd. FIG. 6B shows the activity of sTRSV, vLTSV−, and chimeric ribozymes sTRSV +vLT1, sTRSV+vLT2, and sTRSV+vLT1 & vLT2. FIG. 6C shows the activity of sTRSV, PLMVd, and chimeric ribozymes sTRSV+PL1, sTRSV+PL2, and sTRSV+PL1 & PL2.

FIG. 7 shows the Mg²⁺ dependence of the initial rate (k_{obs}) of sTRSV, PLMVd, sTRSV+PL1, sTRSV+PL2, and sTRSV+PL1 & PL2. K_{obs} is calculated as shown in Example 6.

FIG. 8 depicts a plasmid construct used to test the in vivo activity of various wild type and chimeric ribozymes. The secreted alkaline phosphatase (SEAP) gene is cloned downstream of a cytomegalovirus (CMV) promoter and upstream of a bovine growth hormone 3′ untranslated region (BGH 3′-UTR). The wild-type or chimeric ribozyme coding sequence is inserted into the BGH 3′ UTR between the XbaI and SalI sites.

FIG. 9 shows the in vivo activity of various wild-type and chimeric ribozymes using the construct shown in FIG. 8 in transiently transfected HEK 293 cells.

FIG. 10A shows a schematic structure of the sTRSV hammerhead ribozyme. Each numbered position in loop I and loop II was individually substituted with a cytosine, except for the cytosine at position 5, which was substituted with a guanosine. The circled nucleotides are positions where mutation had a deleterious effect on the initial rate (K_{obs}) of the ribozyme. FIG. 10B shows the initial rate (K_{obs}) of the wild-type and mutant sTRSV hammerhead ribozymes in 50 mM Tris-HCl (pH 7.0) 0.1 mM Mg²⁺.

FIG. 11 shows the structures of Schistozyme 1/1 cis-cleaving hammerhead ribozyme (A) and a I/III trans-cleaving hammerhead ribozyme derived from Schistozyme 1/1 cis-cleaving hammerhead ribozyme (C). Mutations introduced into the bulge within stem I of Schistozyme cis-cleaving hammerhead ribozyme (B) and Schistozyme trans-cleaving hammerhead ribozyme (D) are also shown.

FIG. 12 shows the activity of the Schistozyme cis- and trans-cleaving ribozymes of FIG. 11(A), as-well as the mutants of the Schistozyme cis- and trans-cleaving ribozymes of FIG. 11(B) in 1 mM Mg²⁺ and in 0.1 mM Mg²⁺.

FIG. 13 shows the Mg²⁺ -dependence of the activity of the Schistozyme cis- and trans-cleaving ribozymes and the mutants of the Schistozyme cis- and trans-cleaving ribozymes. The activity is expressed as the % ribozyme cleaved in 1 minute.

FIG. 14 shows certain exemplary schemes for selecting a cis-cleaving hammerhead ribozyme from a library of hammerhead ribozymes having randomized loop sequences. A library of hammerhead ribozymes having randomized sequences in loops I and II is incubated to allow the ribozymes to cis-cleave (I). The cleaved ribozymes are then separated from the uncleaved ribozymes by denaturing polyacrylamide gel electrophoresis (denaturing PAGE), and the cleaved ribozymes are isolated from the gel (II). The cleaved ribozymes are reverse transcribed into cDNA and amplified by PCR (III). During the PCR step, the 5′ fragment that was cleaved from the ribozyme is reintroduced as part of a primer sequence, along with an RNA polymerase promoter for transcribing the PCR products into RNA. The duplex DNA templates are then in vitro transcribed to produce a new library that is enriched for active ribozymes (IV). The duplex DNA templates may also be cloned and sequenced (b). Steps (I) through (IV) may be repeated until the desired level of ribozyme activity is achieved.

FIG. 15 shows certain exemplary schemes for selecting a cis-cleaving hammerhead ribozyme. As described in FIG. 14, a library of hammerhead ribozymes having randomized loop sequences is incubated to allow the ribozymes to cis-cleave (I). The cleaved ribozymes are isolated following separation by denaturing PAGE (II). A synthetic RNA fragment comprising the 5′ fragment that was cleaved from the ribozyme is then ligated to the cleaved ribozymes under conditions that disfavor cleavage (III). The ligated ribozyme is then reverse transcribed and amplified by PCR (IV). A primer used for PCR introduces an RNA polymerase promoter, and the DNA is then transcribed to produce a new library of cis-cleaving ribozymes (V). Steps (I) through (V) may then be repeated until the desired level of cis-cleaving ribozyme activity is achieved.

FIG. 16 shows certain exemplary schemes for selecting a trans-cleaving hammerhead ribozyme. Each ribozyme in the library comprises the sequence of the target RNA as one strand of stems I and III, shown as the region flanked by solid arrows (▾). Each member of the library has randomized sequences in loop II and the bulge within stem I. The library is incubated under conditions that that allow the ribozymes to cleave the targeted RNA sequence (I). The cleaved ribozymes are then isolated from the uncleaved ribozymes following separation by denaturing PAGE. The cleaved ribozymes are reverse transcribed into cDNA and amplified by PCR (III). As described above for FIG. 14, primers used during PCR reintroduce the 5′ sequence that was cleaved from the ribozyme, along with an RNA polymerase promoter. The amplified DNA is then transcribed to produce a new library of hammerhead ribozymes enriched for active ribozymes (IV). The amplified DNA may also be cloned and sequenced (b). Steps (I) through (IV) may be repeated until the desired level of ribozyme activity is achieved.

FIG. 17 depicts schematic representations of certain cis- and trans-cleaving ribozymes. The arrow indicates the cleavage site. FIG. 17A shows a III/III cis-cleaving ribozyme. FIG. 17B shows a I/I cis-cleaving ribozyme. FIG. 17C shows a I/III trans-cleaving ribozyme. FIG. 17D shows a I/II trans-cleaving ribozyme. In FIGS. 17C and 17D, the substrate strand that is cleaved by the trans-cleaving hammerhead ribozyme is represented by a broken line.

FIG. 18 schematically shows the conversion of a III/III cis-cleaving hammerhead ribozyme to either a I/III or a I/II trans-cleaving hammerhead ribozyme according to certain embodiments. FIG. 18A shows a III/III cis-cleaving hammerhead ribozyme. FIG. 18B shows a I/III trans-cleaving hammerhead ribozyme. FIG. 18C shows a I/II trans-cleaving ribozyme.

FIG. 19 shows a schematic representation of Schistosoma mansoni hammerhead ribozyme (Schistozyme) I/I cis-cleaving hammerhead ribozyme and two I/I cis-cleaving hammerhead ribozymes of D. baccettii cricket hammerhead ribozyme (cricketzyme A) and D. schiavazzii cricket hammerhead ribozyme (cricketzyme B).

FIG. 20 schematically shows the conversion of I/I (A) and II/II (B) cis-cleaving hammerhead ribozymes to I/III and II/III trans-cleaving hammerhead ribozymes, respectively.

FIG. 21 shows the activity of two I/III trans-cleaving hammerhead ribozymes at 0.1 mM Mg²⁺ and at 1 mM Mg ²+. Ribozyme A1 has a symmetric bulge within stem I.

FIG. 22 shows a schematic representation of four non-natural trans-cleaving hammerhead ribozymes (TCHRs) derived from Schistozyme I/I cis-cleaving hammerhead ribozyme. TCHR-5:4 contains 5 base pairs in stem III and 4 base pairs after the bulge within stem I. TCHR-7:4 contains 7 base pairs in stem III and 4 base pairs after the bulge within stem I. TCHR-9:4 contains 9 base pairs in stem III and 4 base pairs after the bulge within stem I. TCHR-7:6 contains 7 base pairs in stem III and 6 base pairs after the bulge within stem I.

FIG. 23 shows the activity of TCHR-5:4, TCHR-7:4, TCHR-9:4, and TCHR-7:6 in 50 mM Tris-HCl (pH 7.0) and 0.1 mM MgCl₂.

FIG. 24A shows the initial rates (K_{obs}) for TCHR-5:4, TCHR-7:4, TCHR-9:4, and TCHR-7:6 in 50 mM Tris-HCl (pH 7.0) and 0.1 mM MgCl₂ (dark bars) or 50 mM Tris-HCl (pH 7.0), 0.1 mM MgCl₂, and 0.5 M NaCl (light bars). FIG. 24B shows the K_{obs} values and the activity relative to TCHR-5:4 of each TCHR variant in 50 mM Tris-HCl (pH 7.0) and 0.1 mM MgCl₂. K_{obs} was calculated as described in Example 6.

FIG. 25 shows the activity of TCHR-7:6 and TCHR-9:4 in PBS and 0.1 mM MgCl₂.

FIG. 26 shows a schematic representation of the non-natural I/III trans-cleaving hammerhead ribozyme HH16, which lacks a bulge within stem I.

FIG. 27 shows the activity of TCHR-5:4 and HH-16 in 50 mM Tris-HCl (pH 7.0) and 0.1 mM MgCl₂, in the presence and absence of 0.5 M NaCl.

FIG. 28 shows the activity of TCHR-5:4 and HH-16 in 50 mM MES (2-[N-Morpholino]ethanesulfonic acid) and 10 mM MgCl₂ at pH 5.5 or pH 6.0.

FIG. 29 shows the percent cleaved in one minute of TCHR-5:4 or HH-16 in increasing concentrations of LiCl in 50 mM Tris-HCl (pH 7.0).

FIG. 30 shows the pH dependence of TCHR-5:4 and HH16 in 0.1 mM MgCl₂. FIG. 30A shows the activity of TCHR-5:4 in 50 mM Tris-HCl and 0.1 mM MgCl₂, at various pH levels. FIG. 30B shows the activity of HH16 in 50 mM Tris-HCl and 0.1 mM MgCl₂, at various pH levels. FIG. 30C shows the log of the initial rate (K_{obs}) of TCHR-5:4 and HH16 as a function of pH.

FIG. 31 shows the activity of TCHR-5.4 (A) and HH-16 (B) in Tris-HCl (pH 7.0) and 0.1 mM MgCl₂ at 4, 10, 20, 30, and 37° C. The initial rate (K_{obs}) as a function of temperature for TCHR-5:4 (C) and HH-16 (D) is also shown.

FIG. 32 shows the percent cleaved in 1 minute of TCHR-5:4 and HH-16 in Tris-HCl (pH 7.0), as a function of Mg²⁺ concentration.

FIG. 33 shows an exemplary strategy according to certain embodiments for generating a I/III trans-cleaving hammerhead ribozyme based on Schistozyme hammerhead ribozyme. FIG. 33A shows the Schistozyme hammerhead ribozyme. FIG. 33B shows TCHR-5:4, a I/III trans-cleaving hammerhead ribozyme derived from the Schistozyme. FIG. 33C shows a I/III trans-cleaving hammerhead ribozyme derived from TCHR-5:4, wherein the symmetric bulge within stem I has been converted to an asymmetric bulge. In certain embodiments, an asymmetric bulge allows for selection of the bulge sequence while keeping the substrate RNA sequence constant.

FIG. 34 shows the effect of converting the symmetric bulge of TCHR-7:6 to an asymmetric bulge according to certain embodiments. FIG. 34A shows the structure of TCHR-7:6 (left) and the structure of a mutant TCHR-7:6 having an asymmetric bulge (right). FIG. 34B shows the activity of TCHR-7:6 and the mutant TCHR-7:6 in Tris-HCl (pH 7.0) and 0.1 mM MgCl₂. FIG. 34C shows the same data as in B, except the percent cleavage of the mutant TCHR-7:6 is graphed according to the scale on the right.

FIG. 35 shows a schematic representation of two randomized libraries for isolating I/III trans-cleaving hammerhead ribozymes according to certain embodiments. Library A contains a randomized asymmetric bulge within stem I. Library B contains a randomized loop II.

FIG. 36 shows the oligonucleotide templates A and B used to generate ribozyme library A and ribozyme library B, respectively, in Example 8. T7-A and T7-B are primers that add a T7 promoter (shown in bold) onto the 5′ end of template A and template B, respectively, during PCR amplification of the library. RT-A and RT-B are reverse primers for PCR amplification and may also be used for reverse transcription. Antisense-A and antisense-B are oligonucleotides included in the transcription reaction to prevent cleavage of the ribozymes during transcription.

FIG. 37 shows the activity of the pools of ribozymes obtained from library A (left panel) and library B (right panel) after 3, 5, and 6 rounds of selection.

FIG. 38 shows individual sequences obtained after seven rounds of selection with library A. FIG. 38A shows the full-length ribozyme sequences. The box shows the location of the eight nucleotides of the bulge that were randomized in the original library. The consensus sequence is shown in bold at the bottom. AAAUA is the sequence of loop III. FIG. 38B shows the frequency of each sequence in the pool.

FIG. 39 shows the individual sequences obtained after seven rounds of selection with library B. The box shows the location of the six nucleotides of loop II that were randomized in the original library. The frequency of each hexamer is shown at the bottom.

FIG. 40 shows the activity of several in vitro selected cis-cleaving hammerhead ribozymes. FIG. 40A shows the activity in 50 mM Tris-HCl (pH 7.0) and 0.1 mM MgCl₂ of several individual ribozymes selected from library A. The sequence of the bulge within loop I for each of the ribozymes is shown below the figure. For comparison, the activity of HH2, a cis-cleaving hammerhead ribozyme derived from HH16, is included, along with TCHR-4:5. FIG. 40B shows the activity in 50 mM Tris-HCl (pH 7.0) and 0.1 mM MgCl₂ of several individual ribozymes selected from library B. The loop II sequences of each of the ribozymes is shown below the figure. For comparison, the activity of HH2 and PLMVd are included.

FIGS. 41A and 41B shows schematic diagrams of several trans-cleaving hammerhead ribozymes ((1) and (2)) and cis-cleaving hammerhead ribozymes ((3) - (8)).

FIGS. 42A-D show the sequences of certain naturally-occurring hammerhead ribozymes. Each sequence is broken up to show the location of certain features. The features are labeled as follows: I designates the first strand of stem I, LI designates loop I, I′ designates the second strand of stem I, C designates a portion of the core, II designates the first strand of stem II, LII designates loop I, II′ designates the second strand of stem II, III designates the first strand of stem III, LIII designates loop III, III′ designates the second strand of stem III, and CS designates the cleavage site. For each stem, the “first strand” is the strand that extends 5′ to 3′ from the core and the “second strand” is the strand that extends 5′ to 3′ towards the core. The dashed lines indicate the location of additional sequence that is not depicted in the figure.

FIG. 43A shows the template sequences that were used to transcribe chimeric hammerhead ribozymes in Example 1. FIG. 43B shows the antisense oligos that were included in the transcription reaction to prevent cleavage in Example 1.

### DETAILED DESCRIPTION OF CERTAIN EMBODIMENTS OF THE INVENTION

It is to be understood that both the foregoing general description and the following detailed description are exemplary and explanatory only and are not restrictive of the invention, as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

Standard techniques may be used for recombinant DNA, oligonucleotide synthesis, and tissue culture and transformation (e.g., electroporation lipofection, etc.). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those well known and commonly used in the art. Standard techniques may be used for chemical syntheses, chemical analyses, pharmaceutical preparation, formulation, and delivery, and treatment of patients.

Definitions

Unless otherwise indicated, a “ribozyme” as used herein, is a hammerhead ribozyme.

As used herein, a “hammerhead ribozyme” is comprised of one or more polynucleotides that together function as a phosphodiesterase. In certain embodiments, a hammerhead ribozyme is made up of one polynucleotide. In certain embodiments, a hammerhead ribozyme is made up of two polynucleotides. In certain embodiments, a hammerhead ribozyme is made up of more than two polynucleotides.

A hammerhead ribozyme contains a core, three stems that extend from the core, referred to herein as stem I, stem II, and stem III, and at least one loop, which is located on the opposite end of a stems from the core. In certain embodiments, the ribozyme contains one loop. In certain embodiments, when the ribozyme contains one loop, the loop is located at the end of stem II, and is referred to as loop II. In certain embodiments, a ribozyme contains two loops. In certain embodiments, one of the two loops is located at the end of stem I, and is referred to as loop I, and one of the two loops is located at the end of stem II, and is referred to as loop II.

In certain embodiments, a hammerhead ribozyme comprises RNA or an analog thereof. In certain embodiments, a hammerhead ribozyme comprises RNA or an analog thereof and DNA or an analog thereof. In certain embodiments, the ribozyme core is RNA or an analog thereof. In certain embodiments, one or more of the stems of the ribozyme comprises DNA or an analog thereof.

In certain embodiments, the hammerhead ribozyme core comprises the polynucleotide sequence 5′- . . . UAH . . . WYGANGA . . . GAAA . . . -3′, wherein H is selected from A, C, and U; W is selected from C, U, and A; Y is selected from C and U; and N is selected from A, C, G, and U. The “cleavage site” of the hammerhead ribozyme is shown as a carat (ˆ) mark. The ellipses represent intervening portions of the ribozyme that are not part of the core. In certain embodiments, the ellipses represent the stems of the hammerhead ribozyme.

In certain embodiments, the core and intervening portions are comprised of one polynucleotide. In certain embodiments, the core and intervening portions are comprised of more than one polynucleotide. In certain embodiments, the core is comprised of two polynucleotides. In certain embodiments, the core is comprised of polynucleotide 5′- . . . UAH . . . -3′ and polynucleotide 5′- . . . WYGANGA . . . GAAA . . . -3′. In certain embodiments, the core is comprised of polynucleotide 5′- . . . GAAA . . . UAH . . . -3′ and polynucleotide 5′- . . . WYGANGA . . . .3′.

In certain embodiments, the core contains the sequence 5′-WYGANGAN-3′ or 5′-WYGANGANN-3′, where each N is a nucleotide and each N may the same or different. In certain embodiments, the core contains the sequence 5′-NGAAA-5′, where N is a nucleotide.

As used herein, “complementary” refers to a nucleotide or nucleotide sequence that hybridizes to a given nucleotide or nucleotide sequence. For instance, for DNA, the nucleotide A is complementary to T and vice versa, and the nucleotide C is complementary to G and vice versa. For instance, in RNA, the nucleotide A is complementary to the nucleotide U and vice versa, and the nucleotide C is complementary to the nucleotide G and vice versa. Complementary nucleotides include those that undergo Watson and Crick base pairing and those that base pair in alternative modes. For instance, as used herein for RNA, the nucleotide G is complementary to the nucleotide U and vice versa, and the nucleotide A is complementary to the nucleotide G and vice versa. Therefore, in an RNA molecule, the complementary base pairs are A and U, G and C, G and U, and A and G. Other combinations, e.g., A and C or C and U, are considered to be non-complementary base pairs.

A complementary sequence is comprised of individual nucleotides that are complementary to the individual nucleotides of a given sequence, where the complementary nucleotides are ordered such that they will pair sequentially with the nucleotides of the given sequence. Such a complementary sequence is said to be the “complement” of the given sequence. For example, complements of the given sequence, 5′-ACUAGUC-3′, include 3′-UGAUCAG-5′ and 3′-UGGACGG-3′, among others. In the latter sequence, the third and sixth base pairs are both non-Watson and Crick G/U complementary base pairs.

A “stem” as used herein, is a nucleic acid motif that extends from the ribozyme core, at least a portion of which is double-stranded. In certain embodiments, there is a loop at the opposite end of the stem from the ribozyme core, and this loop connects the two strands of the double-stranded stem. In certain embodiments, a stem comprises 2 to 20 complementary base pairs. In certain embodiments, a stem comprises 2 to 10 complementary base pairs. In certain embodiments, a stem comprises 3, 4, 5, 6, 7, 8, or 9 complementary base pairs.

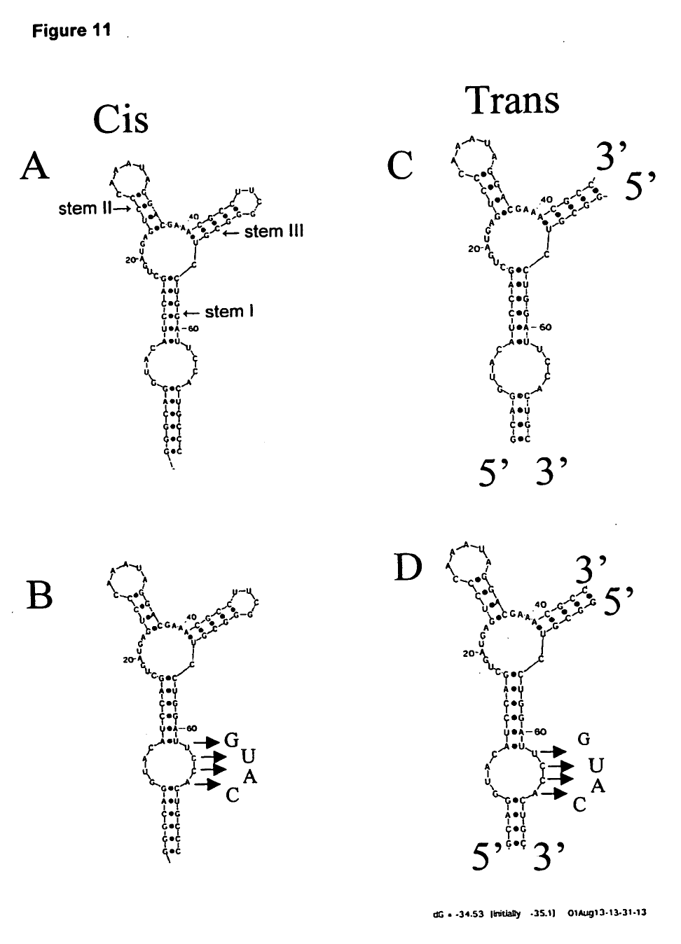

Stems are numbered according to where they extend from the core sequence. In certain embodiments, a hammerhead ribozyme contains three stems, which are referred to as stem I, stem II, and stem III. In certain embodiments, stem I extends from the core between the sequence UAH and the sequence WYGANGA. In certain embodiments, stem II extends from the core between the sequence WYGANGA and the sequence GAAA. In certain embodiments, stem III extends from the core between the sequence GAAA and the sequence UAH. Thus, in certain embodiments, a ribozyme may be configured as follows: 5′-[first strand of stem III] UAH [first strand of stem I] . . . [second strand of stem I] WYGANGA [first strand of stem II] . . . [second strand of stem II] GAAA [second strand of stem III]-3′. The ellipses in this example represent loop sequences that connect the first and second strands of stem I and stem II. Another exemplary configuration for a ribozyme according to certain embodiments is shown in FIG. 11A. Although the loops are located at the ends of stem II and stem III rather than stem I and stem II, the labeling of the stems relative to the core remains the same as described above.

As used herein, a “bulge” is a sequence of nucleotides that is not paired with another strand and is flanked on both sides by double-stranded nucleic acid sequences. In certain embodiments, a bulge is located within a stem. When a bulge is located within a stem, the nucleotides of the bulge are considered to be part of the stem. In certain embodiments, the bulge is located within stem I. In certain embodiments, the bulge is located within stem II. In certain embodiments, a hammerhead ribozyme comprises more than one bulge. In certain embodiments, a bulge within a stem is located two base pairs from the core. In certain embodiments, a bulge within a stem is located three base pairs from the core. In certain embodiments, a bulge within a stem is located four base pairs from the core. In certain embodiments, a bulge within a stem is located five base pairs from the core. In certain embodiments, a bulge within a stem is located six base pairs from the core. In certain embodiments, a bulge within a stem is located seven base pairs from the core. In certain embodiments, a bulge within a stem is located eight base pairs from the core.

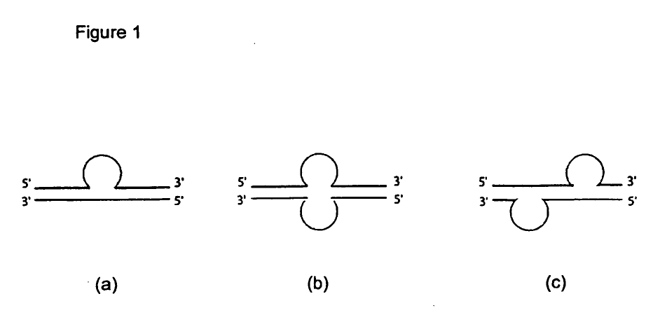

In certain embodiments, one or both strands of the stem contains a bulge. In certain embodiments, only one strand of the stem contains a bulge. When only one strand of the stem contains a bulge, the bulge is referred to as an “asymmetric bulge,” a non-limiting example of which is shown In FIG. 1(a). In certain embodiments, an asymmetric bulge may comprise 1 to 20 nucleotides. In certain embodiments, an asymmetric bulge may comprise 2 to 10 nucleotides. In certain embodiments, an asymmetric bulge may comprise 3 to 8 nucleotides.

When both strands of the stem contain bulges, and the bulges are opposite each other, the bulge is referred to as a “symmetric bulge,” a non-limiting example of which is shown in FIG. 1(b). In certain embodiments, a symmetric bulge may comprise 1 to 20 nucleotides on each strand, so that a total of 2 to 40 nucleotides of the stem are contained in the symmetric bulge. In certain embodiments, a symmetric bulge contains a total of 2 to 20 nucleotides. In certain embodiments, a symmetric bulge contains a total of 4 to 16 nucleotides. The number of nucleotides from each strand in a symmetric bulge need not be equal. For example, one strand of the stem may have a 6 nucleotide bulge, while the second strand of the stem may have an 8 nucleotide bulge opposite the 6 nucleotide bulge. In that example, the symmetric bulge contains 14 nucleotides.

When both strands contain bulges, but the bulges are not opposite each other, then each strand is said to contain an asymmetric bulge, and the stem is said to contain two asymmetric bulges, a non-limiting example of which is shown in FIG. 1(c).

In certain embodiments, at least 30% of the nucleotides in a stem are part of a complementary base pair. The remaining base pairs may be mismatched, non-complementary base pairs, or may be part of a bulge. In certain embodiments, at least 40% of the nucleotides in a stem are part of a complementary base pair. In certain embodiments, at least 50% of the nucleotides in a stem are part of a complementary base pair. In certain embodiments, at least 60% of the nucleotides in a stem are part of a complementary base pair. In certain embodiments, at least 70% of the nucleotides in a stem are part of a complementary base pair. In certain embodiments, at least 80% of the nucleotides in a stem are part of a complementary base pair. In certain embodiments, at least 90% of the nucleotides in a stem are part of a complementary base pair. In certain embodiments, at least 95% of the nucleotides in a stem are part of a complementary base pair. In certain embodiments, at least 99% of the nucleotides in a stem are part of a complementary base pair. In certain embodiments, 100% of the nucleotides in a stem are part of a complementary base pair.

As used herein, a “loop” is a sequence of nucleotides that is not paired with another strand and is located at the distal end of a stem that is opposite the core. In certain embodiments, a loop is between 1 to 20 nucleotides long. In certain embodiments, a loop is between 2 and 10 nucleotides long. In certain embodiments, a loop is between 3 and 8 nucleotides long. The loop is numbered according to the stem to which it is attached. Therefore, loop I is located at the end of stem I opposite the core, loop II is located at the end of stem II opposite the core, and loop III is located at the end of stem III opposite the core.

As used herein, a “stem/loop” refers to the entire stem, along with any bulges within that stem, and the loop at the end of the stem. For example, stem/loop I includes stem I, including any bulges within stem I, and loop I. If a stem, lacks a loop, then stem/loop refers to the stem, along with any bulges within that stem.

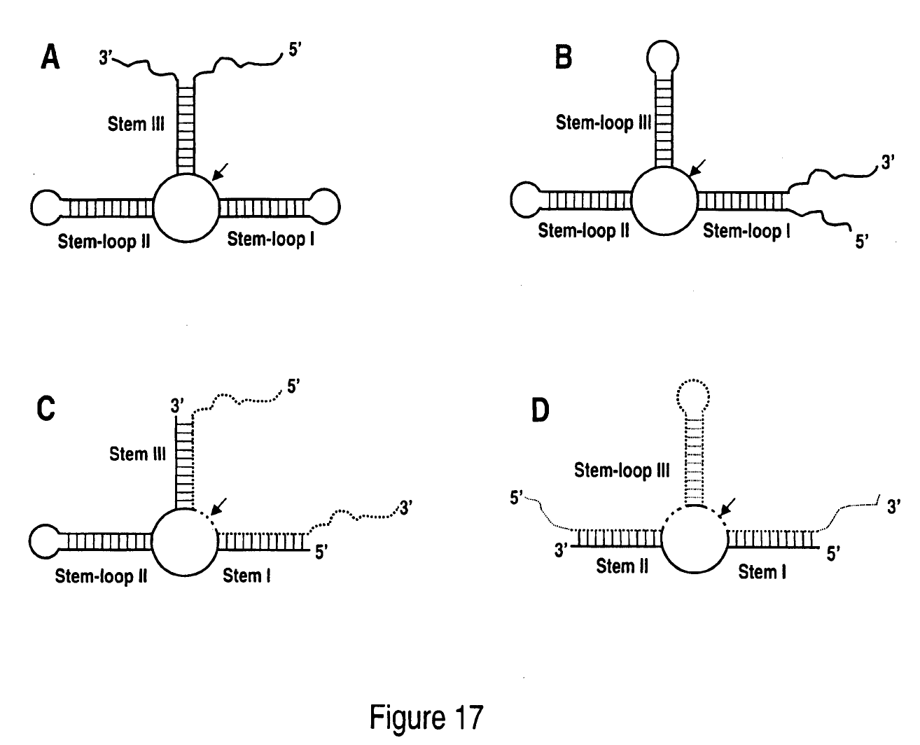

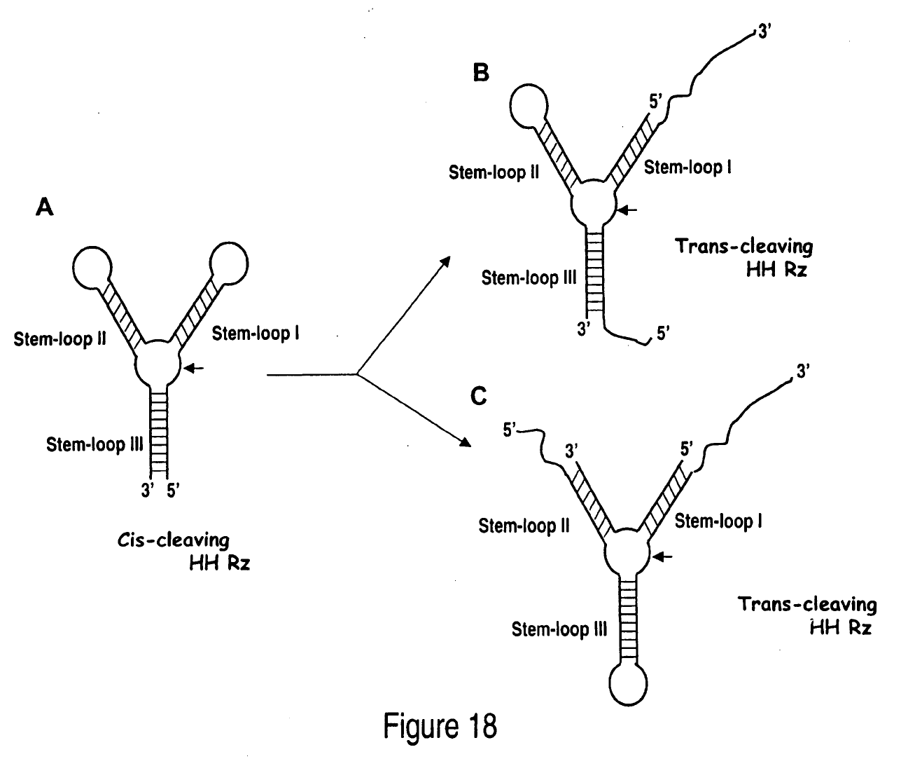

As used herein, a “cis-cleaving hammerhead ribozyme” is a hammerhead ribozyme that, prior to cleavage, is comprised of a single polynucleotide. A cis-cleaving hammerhead ribozyme is capable of cleaving itself. Nonlimiting exemplary cis-cleaving hammerhead ribozymes are shown in FIGS. 17A and 17B. In certain embodiments, the 5′ and 3′ termini of the polynucleotide that comprises the cis-cleaving hammerhead ribozyme are located at the end of stem III. See, e.g., FIG. 17A. In certain embodiments, the 5′ and 3′ termini of the polynucleotide that comprises the cis-cleaving hammerhead ribozyme are located at the end of stem I. See, e.g., FIG. 17B.

As used herein, a “trans-cleaving hammerhead ribozyme” is a hammerhead ribozyme that, prior to cleavage, is comprised of at least two polynucleotides.

In certain embodiments, a trans-cleaving ribozyme is comprised of two polynucleotides. In certain embodiments, one of the polynucleotides comprises, 5′ to 3′, one strand of stem III, part of the core including the cleavage site, and one strand of stem I, and a second polynucleotide comprises, 5′ to 3′, the other strand of stem I, part of the core, stem II, loop II, another part of the core, and the other strand of stem III. Such a trans-cleaving hammerhead ribozyme is referred to as a “I/III trans-cleaving hammerhead ribozyme” or a “I/III trans-cleaving ribozyme.” See, e.g., the nonlimiting embodiment in FIG. 17C. In certain embodiments, one of the polynucleotides comprises, 5′ to 3′, one strand of stem I, part of the core, and one strand of stem II, and a second polynucleotide comprises, 5′ to 3′, the other strand of stem II, another part of the core, stem III, loop III, part of the core including the cleavage site, and the other strand of stem I. Such a trans-cleaving hammerhead ribozyme is referred to as a “I/II trans-cleaving hammerhead ribozyme” or a “I/II trans-cleaving ribozyme.” See, e.g., the nonlimiting embodiment in FIG. 17D.

In certain embodiments, the term “trans-cleaving hammerhead ribozyme” refers to the polynucleotide or polynucleotides of the ribozyme that are not cleaved. In certain such embodiments, the strand that is cleaved by the ribozyme may be referred to as the “substrate” strand. In certain embodiments, the substrate strand is a target RNA. In various embodiments, the target RNA may be any RNA, including, but not limited to, any RNA found in a cell, such as an mRNA, a tRNA, an snRNA, etc. In various embodiments, target RNA may be an RNA in vitro, including RNAs normally found in a cell, and RNAs created in vitro, such as a synthetic RNA or an in vitro transcribed RNA.

As used herein, the term “non-natural” hammerhead ribozyme refers to a hammerhead ribozyme sequence that does not occur in its entirety in nature.

The term “nucleotide”, as used herein, refers to naturally- and non-naturally- occurring nucleotides and nucleotide analogs. Nucleotides include, but are not limited to, adenosine, cytosine, guanosine, thymidine, uracil, 4-acetylcytosine, 8-hydroxy-N6-methyladenosine, aziridinyl-cytosine, pseudoisocytosine, 5-(carboxyhydroxylmethyl) uracil, 5-fluorouracil, 5-bromouracil, 5-carboxymethylaminomethyl-2-thiouracil, 5-carboxy-methylaminomethyluracil, dihydrouracil, inosine, N6-iso-pentenyladenine, 1-methyladenine, 1-methylpseudouracil, 1-methylguanine, 1-methylinosine, 2,2-dimethyl-guanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, 5′-methoxycarbonyl-methyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine and 2,6-diaminopurine.

The terms “nucleic acid,” “nucleic acid sequence,” “nucleic acid molecule,” and “polynucleotide” refer to a DNA sequence or analog thereof, or an RNA sequence or analog thereof. Nucleic acids are formed from nucleotides, including, but not limited to, the nucleotides listed above.

As used herein, “low Mg²⁺” concentration of less than about 1 mM. In certain embodiments, the Mg²⁺ concentration is less than about 0.5 mM Mg²⁺. In certain embodiments, the Mg²⁺ concentration is less than about 0.1 mM Mg²⁺.

As used herein, the “activity” of a ribozyme may be determined by any of the following methods: (a) initial rate of cleavage (k_{obs}), which is calculated in certain embodiments using the method described in McCaffrey et al. (1999) in Intracellular Ribozyme Applications: Principles and Protocols, Horizon Scientific Press, pp. 57-68, which is incorporated herein by reference for any purpose; (b) the percent ribozyme cleaved in 1 minute under certain conditions; and/or (c) the maximum percent of ribozymes cleaved under certain conditions in a defined amount of time.

As used herein, “inserted in frame” means that a first nucleic acid sequence is inserted into a second nucleic acid sequence such that it does not alter the reading frame of a coding region of the second nucleic acid sequence. In certain embodiments, the first nucleic acid sequence is of a length that is a multiple of three, so that it encodes an integer number of amino acids. In certain embodiments, the first nucleic acid sequence, once inserted into the second nucleic acid sequence, does not contain any stop codons in the coding frame of the second nucleic acid sequence.

Certain Exemplary Embodiments of the Invention

Certain exemplary embodiments of the invention relate to non-natural cis- and trans-cleaving hammerhead ribozymes. In certain embodiments, the invention relates to hammerhead ribozymes that cleave in the presence of low concentrations of Mg²⁺.

In certain embodiments, the invention relates to methods of making non-natural trans-cleaving hammerhead ribozymes by selecting cis-cleaving hammerhead ribozymes and converting them to trans-cleaving hammerhead ribozymes.

In certain embodiments, the invention relates to methods of cleaving a target RNA in vivo. In certain embodiments, the invention relates to methods of reducing protein expression in vivo. In certain embodiments, the invention relates to methods of reducing the level of a target mRNA in vivo. In certain embodiments, the invention relates to pharmaceutical compositions comprising the ribozymes. In certain embodiments, the invention relates to pharmaceutical compositions comprising polynucleotides encoding ribozymes.

In certain embodiments, a putative tertiary interaction between loop I and loop II of a hammerhead ribozyme results in greater activity at lower Mg²⁺ concentrations. In certain embodiments, a putative tertiary interaction between loop I and a bulge within loop II of a hammerhead ribozyme results in greater activity at lower Mg²⁺ concentrations. In certain embodiments, a putative tertiary interaction between loop II and a bulge within loop I of a hammerhead ribozyme results in greater activity at lower Mg²⁺ concentrations. In certain embodiments, a putative tertiary interaction between a bulge within loop I and a bulge within loop II of a hammerhead ribozyme results in greater activity at lower Mg²⁺ concentrations. In certain embodiments, mutations within loop I or a bulge within stem I of a hammerhead ribozyme can be compensated for by mutations in loop II or a bulge within stem II. In certain embodiments, hammerhead ribozymes that undergo putative interactions may be either cis-cleaving hammerhead ribozymes or trans-cleaving hammerhead ribozymes.

In certain embodiments, hammerhead ribozymes cleave in the presence of less than 1 mM Mg²⁺. In certain embodiments, hammerhead ribozymes-cleave in the presence of less than 0.5 mM Mg²⁺. In certain embodiments, hammerhead ribozymes cleave in the presence of 0.1 mM Mg²⁺.

In certain embodiments, at least 20% of cis-cleaving or trans-cleaving ribozymes are cleaved when incubated at 37° C. in 50 mM Tris (pH 7.0) and 1 mM MgCl₂ for 30 minutes. In certain embodiments, at least 30% of cis-cleaving or trans-cleaving ribozymes are cleaved when incubated at 37° C. in 50 mM Tris (pH 7.0) and 1 mM MgCl₂ for 30 minutes. In certain embodiments, at least 40% of cis-cleaving or trans-cleaving ribozymes are cleaved when incubated at 37° C. in 50 mM Tris (pH 7.0) and 1 mM MgCl₂ for 30 minutes. In certain embodiments, at least 50% of cis-cleaving or trans-cleaving ribozymes are cleaved when incubated at 37° C. in 50 mM Tris (pH 7.0) and 1 mM MgCl₂ for 30 minutes. In certain embodiments, at least 75% of cis-cleaving or trans-cleaving ribozymes are cleaved when incubated at 37° C. in 50 mM Tris (pH 7.0) and 1 mM MgCl₂ for 30 minutes. In certain embodiments, at least 90% of cis-cleaving or trans-cleaving ribozymes are cleaved when incubated at 37° C. in 50 mM Tris (pH 7.0) and 1 mM MgCl₂ for 30 minutes.

In certain embodiments, at least 20% of cis-cleaving or trans-cleaving ribozymes are cleaved when incubated at 37° C. in 50 mM Tris (pH 7.0) and 0.1 mM MgCl₂ for 30 minutes. In certain embodiments, at least 30% of cis-cleaving or trans-cleaving ribozymes are cleaved when incubated at 37° C. in 50 mM Tris (pH 7.0) and 0.1 mM MgCl₂ for 30 minutes. In certain embodiments, at least 40% of cis-cleaving or trans-cleaving ribozymes are cleaved when incubated at 37° C. in 50 mM Tris (pH 7.0) and 0.1 mM MgCl₂ for 30 minutes. In certain embodiments, at least 50% of cis-cleaving or trans-cleaving ribozymes are cleaved when incubated at 37° C. in 50 mM Tris (pH 7.0) and 0.1 mM MgCl₂ for 30 minutes. In certain embodiments, at least 75% of cis-cleaving or trans-cleaving ribozymes are cleaved when incubated at 37° C. in 50 mM Tris (pH 7.0) and 0.1 mM MgCl₂ for 30 minutes. In certain embodiments, at least 90% of cis-cleaving or trans-cleaving ribozymes are cleaved when incubated at 37° C. in 50 mM Tris (pH 7.0) and 0.1 mM MgCl₂ for 30 minutes.

In certain embodiments, a ribozyme has a k_{obs} of at least 0.1 min⁻¹ at 37° C. in buffer comprising 50 mM Tris-HCl (pH 7.0) and 1 mM MgCl₂. In certain embodiments, a ribozyme has a k_{obs} of at least 0.5 min⁻¹ at 37° C. in buffer comprising 50 mM Tris-HCl (pH 7.0) and 1 mM MgCl₂. In certain embodiments, a ribozyme has a k_{obs} of at least 1 min⁻¹ at 37° C. in buffer comprising 50 mM Tris-HCl (pH 7.0) and 1 mM MgCl₂. In certain embodiments, a ribozyme has a k_{obs} of at least 0.1 min⁻¹ at 37° C. in buffer comprising 50 mM Tris-HCl (pH 7.0) and 0.5 mM MgCl₂. In certain embodiments, a ribozyme has a k_{obs} of at least 0.5 min⁻¹ at 37° C. in buffer comprising 50 mM Tris-HCl (pH 7.0) and 0.5 mM MgCl₂. In certain embodiments, a ribozyme has a k_{obs} of at least 1 min⁻¹ at 37° C. in buffer comprising 50 mM Tris-HCl (pH 7.0) and 0.5 mM MgCl₂. In certain embodiments, a ribozyme has a k_{obs} of at least 0.1 min⁻¹ at 37° C. in buffer comprising 50 mM Tris-HCl (pH 7.0) and 0.1 mM MgCl₂. In certain embodiments, a ribozyme has a k_{obs} of at least 0.5 min⁻¹ at 37° C. in buffer comprising 50 mM Tris-HCl (pH 7.0) and 0.1 mM MgCl₂. In certain embodiments, a ribozyme has a k_{obs} of at least 1 min⁻¹ at 37° C. in buffer comprising 50 mM Tris-HCl (pH 7.0) and 0.1 mM MgCl₂.

Certain naturally-occurring hammerhead ribozymes function as cis-cleaving hammerhead ribozyme. Certain naturally-occurring ribozymes have much greater activity than certain synthetic ribozymes that have been developed in the lab, especially at low Mg²⁺ concentrations. In certain embodiments, non-natural cis-cleaving hammerhead ribozymes are provided that have activity comparable to certain naturally-occurring hammerhead ribozymes, even at low Mg²⁺ concentrations.

In certain embodiments, the invention provides non-natural chimeric cis-cleaving hammerhead ribozymes. In certain embodiments, chimeric hammerhead ribozymes are made by replacing one or more loop sequences of a first hammerhead ribozyme with the corresponding loop sequences from a second hammerhead ribozyme. In certain embodiments, chimeric hammerhead ribozymes are made by replacing one or more bulge sequences of a first hammerhead ribozyme with the corresponding bulge sequences from a second hammerhead ribozyme. In certain embodiments, chimeric hammerhead ribozymes are made by replacing one or more stem sequences of a first hammerhead ribozyme with the corresponding stem sequences from a second hammerhead ribozyme. In certain embodiments, chimeric hammerhead ribozymes are made by replacing one or more stem/loop sequences of a first hammerhead ribozyme with the corresponding stem/loop sequences from a second hammerhead ribozyme. In certain chimeric ribozyme embodiments, one or both ribozymes are naturally-occurring hammerhead ribozymes. In certain chimeric ribozyme embodiments, one or both ribozymes are non-natural hammerhead ribozymes.

In certain embodiments, loop I and loop II of a first hammerhead ribozyme are replaced with loop I and loop II from a second hammerhead ribozyme. In certain embodiments, replacing both of the loops of a first hammerhead ribozyme with both of the loops of a second hammerhead ribozyme results in a chimeric ribozyme that has greater activity than a chimeric ribozyme with only one of the loops replaced. In certain embodiments, one or more stems and/or loops of a first hammerhead ribozyme selected from cherry small circular RNA+ (Scc+), cherry small circular RNA− (Scc−), Lucerne transient streak virusoid+ (sLTSV+), Lucerne transient streak virusoid− (sLTSV−), Tobacco ringspot virus satellite RNA+ (sTRSV+), Arabis mosaic virus (sArMV), Chicory yellow mottle virus satellite RNA (sCYMV), Barley yellow dwarf virus satellite RNA− (sBYDV−), Barley yellow dwarf virus satellite RNA+ (sBYDV+), Peach latent mosaic virus RNA+ (PLMVd+), Peach latent mosaic virus RNA− (PLMVd−), Chrysanthemum chlorotic mottle viroid+ (CChMVd+), Chrysanthemum chlorotic mottle viroid− (CChMVd−), Subterraneum clover mottle virusoid (vSCMoV), and velvet tobacco mottle virusoid (vVTMoV) replace one or more stems and/or loops of a second hammerhead ribozyme, where the first and second hammerhead ribozymes are different.

In certain embodiments, loop II and a bulge within stem I of a first hammerhead ribozyme are replaced with loop II and a bulge within stem I from a second hammerhead ribozyme. In certain embodiments, replacing loop II and the bulge within stem I of a first hammerhead ribozyme with loop II and the bulge within stem I of a second hammerhead ribozyme results in a chimeric ribozyme that has greater activity than a chimeric ribozyme with only one of those regions replaced. In certain embodiments, loop II and/or the bulge within stem I of a first hammerhead ribozyme selected from Notophthalmus viridescens satellite RNA (newt), Ambystoma talpoideum (Am. ta.), Amphiuma tridactylum (Am. tr.), Schistosoma mansoni hammerhead ribozyme (Schistozyme), D. baccettii cricket hammerhead ribozyme (cricketzyme A), D. schiavazzii cricket hammerhead ribozyme (cricketzyme B), and Avocado sunblotch viroid+ (ASBV+), replace loop II and/or the bulge within stem I of a second hammerhead ribozyme, where the first and second hammerhead ribozymes are different.

In certain embodiments, loop I and a bulge within stem II of a first hammerhead ribozyme are replaced with loop I and a bulge within stem II from a second hammerhead ribozyme. In certain embodiments, replacing loop I and the bulge within stem II of a first hammerhead ribozyme with the bulge within stem II and loop I of a second hammerhead ribozyme results in a chimeric ribozyme that has greater activity than a chimeric ribozyme with only one of those regions replaced. In certain embodiments, loop I and/or the bulge within stem II of a first hammerhead ribozyme selected from Chrysanthemum chlorotic mottle viroid− (CChMVd−) and Barley yellow dwarf virus satellite RNA (sBYDV+) replace loop I and/or the bulge within stem II of a-second hammerhead ribozyme, where the first and second hammerhead ribozymes are different.

In certain embodiments, a bulge within stem I and a bulge within stem II of a first hammerhead ribozyme are replaced with a bulge within stem I and a bulge within stem II from a second hammerhead ribozyme. In certain embodiments, replacing the bulge within stem I and the bulge within stem II of a first hammerhead ribozyme with the bulge within stem I and the bulge within stem II of a second hammerhead ribozyme results in a chimeric ribozyme that has greater activity than a chimeric ribozyme with only one of those regions replaced. In certain embodiments, the bulge within stem I and/or the bulge within stem II of a first hammerhead ribozyme selected from Avocado sunblotch viroid− (ASBV−) and Carnation small viroid-like RNA+ (CarSV+) replace the bulge within stem I and/or the bulge within stem II of a second hammerhead ribozyme, where the first and second hammerhead ribozymes are different.

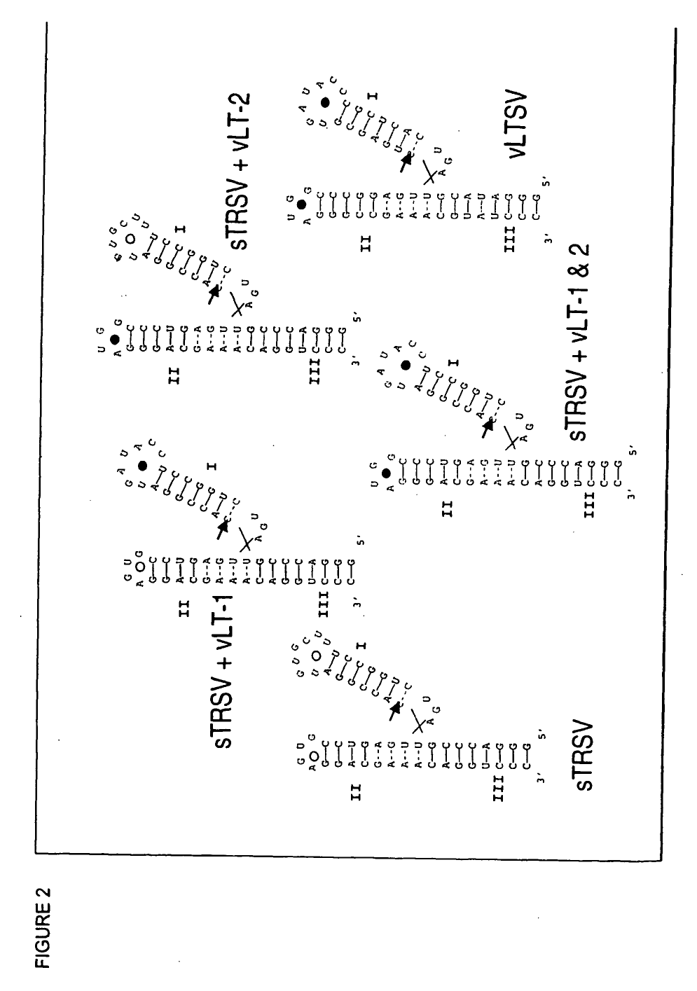

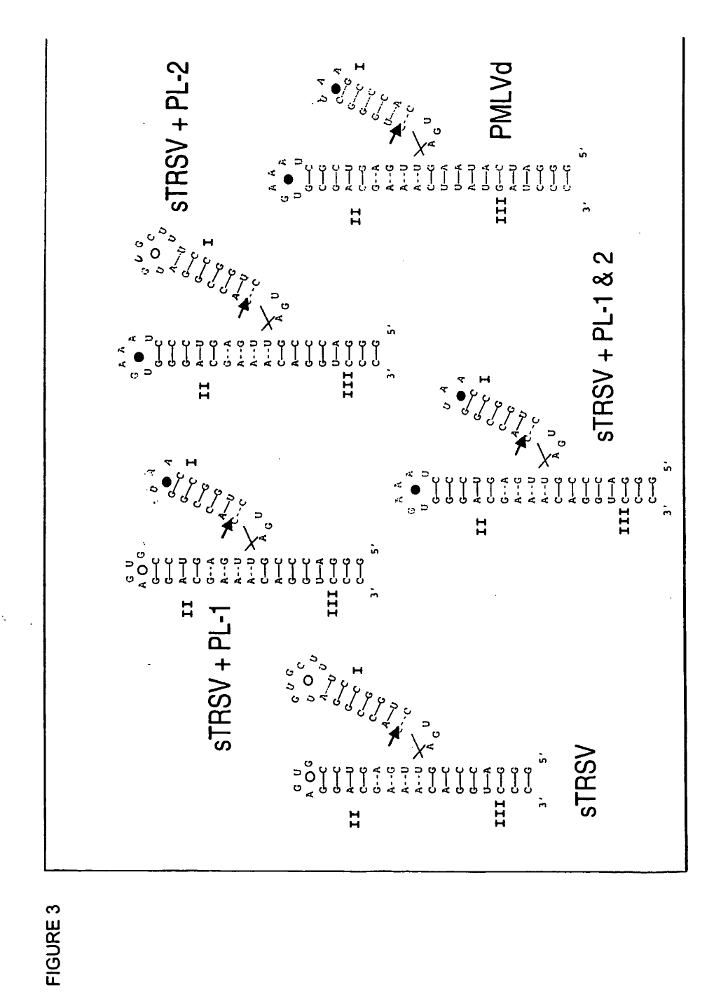

Certain nonlimiting examples of such chimeric cis-cleaving hammerhead ribozymes are shown in FIGS. 2 and 3.

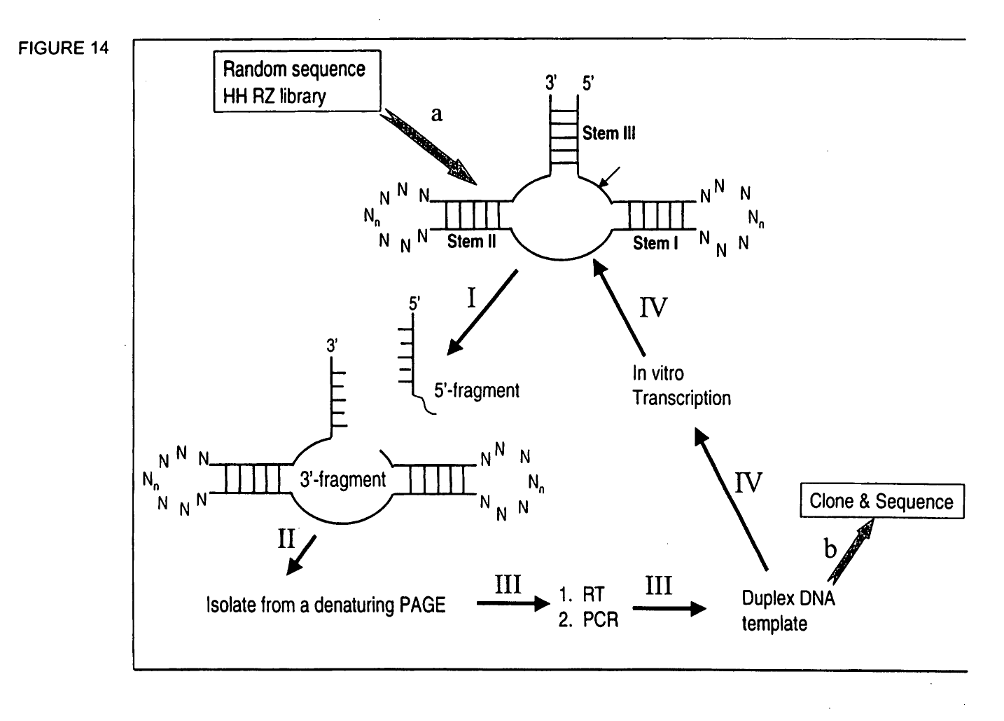

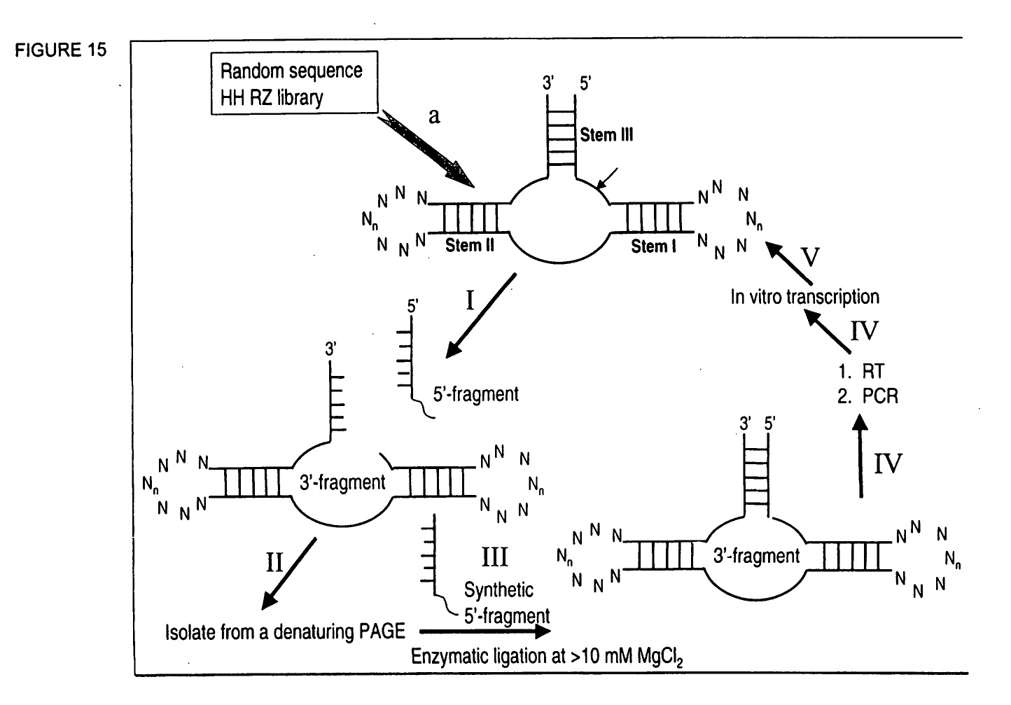

In certain embodiments, non-natural cis-cleaving hammerhead ribozymes can be selected in vitro. Two nonlimiting exemplary selection schemes are shown in FIGS. 14 and 15. In certain embodiments, a library of ribozymes is made in which loop I is randomized across the members of the library. In certain embodiments, a library of ribozymes is made in which loop II is randomized across the members of the library. In certain embodiments, a library of ribozymes is made in which loop I and loop II are randomized across the members of the library. In certain embodiments, the core, stem I, stem II, and stem III are constant across the members of the library. In certain embodiments, either loop I or loop II is also constant across the members of the library.

In certain embodiments, a randomized loop is between 2 and 20 nucleotides long. In certain embodiments, a randomized loop is between 3 and 10 nucleotides long. In certain embodiments, a randomized loop is between 3 and 8 nucleotides long. In certain embodiments, a randomized loop is 3, 4, 5, 6, 7, or 8 nucleotides long.

In certain embodiments, a library of ribozymes having constant core and stem sequences and one or more randomized loop sequences is synthesized by in vitro transcription. In certain embodiments, a library of ribozymes is chemically synthesized. Chemical RNA synthesis is described, e.g., in Scaringe, S. A. et al. (1998) J. Am. Chem. Soc., 120:11820-11821.

In certain embodiments, the library of ribozymes is incubated under conditions allowing cleavage of active ribozymes, and those ribozymes that have cleaved are separated from substantially all ribozymes that have not cleaved. In certain embodiments, incubation of the ribozyme library occurs at 37° C. in buffer comprising 50 mM Tris-HCl (pH 7.0) and 0.1 mM MgCl₂. In certain embodiments, the ribozymes that have cleaved are separated from substantially all of the ribozymes that have not cleaved on the basis of size and/or charge. In certain embodiments, the separating step is denaturing polyacrylamide gel electrophoresis, or denaturing PAGE.

In certain embodiments, following separation of the ribozymes that cleaved from substantially all of the ribozymes that have not cleaved, the cleaved ribozymes are isolated, reverse transcribed, and amplified by PCR. In certain embodiments, one of the primers used for PCR adds back the sequence that was cleaved from the ribozyme, and one of the primers adds a promoter sequence for transcribing the PCR product into RNA. In certain embodiments, the promoter sequence is a promoter for an RNA polymerase. In certain embodiments, any suitable RNA polymerases may be used, including, but not limited to, T7 RNA polymerase, SP6 RNA polymerase, and Qβ-replicase. In certain embodiments, following PCR amplification of the ribozymes, the PCR products are cloned into a vector, transformed into bacteria, and individual clones are isolated and sequenced.

In certain embodiments, the PCR-amplified ribozyme sequences are transcribed and the selection process is repeated. In certain embodiments, the selection process is repeated 1 to 20 times. In certain embodiments, the selection process is repeated 3 to 10 times. In certain embodiments, the selection process is repeated 5, 6, or 7 times.

In certain embodiments, at least one of the stems of the non-natural cis-cleaving hammerhead ribozyme includes an aptamer sequence that is capable of binding a small molecule. For a discussion of nonlimiting exemplary aptamer sequences and small molecules, see, e.g., published PCT sequence is part of stem II. In certain embodiments, binding of the small molecule affects ribozyme activity. In certain embodiments, binding of the small molecule activates ribozyme activity. In certain embodiments, binding of the small molecule represses ribozyme activity.

In certain embodiments, a polynucleotide encoding a non-natural cis-cleaving hammerhead ribozyme is inserted into a gene. In certain embodiments, a polynucleotide encoding a ribozyme is inserted into an exon, an intron, a 5′-untranslated region, or a 3′-untranslated region of a gene. In certain embodiments, a polynucleotide encoding a ribozyme is inserted in frame into an exon of a gene. In certain embodiments, the gene is inserted into a vector. In certain embodiments, the vector is introduced into a host cell. Certain suitable methods of introduction include, but are not limited to, transformation, transfection, transduction, passive uptake, and conjugation-induced (see, e.g., Hermanson, G T (1995) Bioconjugate Techniques, Academic Press). Suitable host cells include, but are not limited to, bacteria cells, yeast cells, insect cells, mouse cells, rat cells, monkey cells, human cells, etc. In certain embodiments, following transcription of the gene, the ribozyme cleaves the mRNA. In certain embodiments, cleavage of the mRNA results in reduced mRNA levels and/or reduced levels of the protein encoded by the gene.

In certain embodiments, the ribozyme inserted into the gene contains an aptamer sequence capable of binding a small molecule. In certain embodiments, cleavage of the mRNA transcribed from the gene in vivo is affected by incubation of the host cell with the small molecule that binds to the aptamer. In certain embodiments, cleavage of the mRNA is increased by incubation with the small molecule. In certain embodiments, cleavage of the mRNA is decreased by incubation with the small molecule.

In certain embodiments, a polynucleotide sequence encoding a non-natural cis-cleaving hammerhead ribozyme in inserted into a gene in a vector, and the vector is administered to an animal. Suitable vectors include, but are not limited to, viral vectors, including retroviruses, adenoviruses, and lentiviruses. In certain embodiments, the gene containing the ribozyme-encoding polynucleotide sequence is inserted into an animal, such as a mouse, by standard transgenic methods. Such methods are described, e.g., in Transgenic Animals; Academic Press, Inc. (1992); ISBN: 0123045304. In certain embodiments, the gene containing the ribozyme-encoding polynucleotide sequence is administered into an animal using a virus. Suitable methods of administration using viruses are described, e.g., in Gene Therapy Technologies, Applications and Regulations: From Laboratory to Clinic; John Wiley & Sons (1999); ISBN: 0471967092.

As discussed above, trans-cleaving hammerhead ribozymes are ribozymes that are comprised of two or more polynucleotides. In certain embodiments, trans-cleaving hammerhead ribozymes comprise a core, three stems, and may or may not have one or more loops. In certain embodiments, two or more separate polynucleotides contribute to the core of a trans-cleaving hammerhead ribozyme. Trans-cleaving hammerhead ribozymes may exist in several different configurations. Two nonlimiting exemplary trans-cleaving hammerhead ribozymes, each containing two separate polynucleotides, are shown in FIGS. 17C and 17D. FIG. 17C shows an exemplary I/III trans-cleaving hammerhead ribozyme. FIG. 17D shows an exemplary I/II trans-cleaving hammerhead ribozyme. In each case, the substrate strand, which is the strand that is cleaved, is shown as a broken line.

In certain embodiments, trans-cleaving hammerhead ribozymes may be converted from cis-cleaving hammerhead ribozymes by removing one of the loops of the cis-cleaving hammerhead ribozyme and creating polynucleotide termini where the loop was. A nonlimiting exemplary conversion of a cis-cleaving hammerhead ribozyme into a trans-cleaving hammerhead ribozyme is shown in FIGS. 17A and 17C, where loop I of the cis-cleaving hammerhead ribozyme was removed to create the trans-cleaving hammerhead ribozyme of FIG. 17C. Another nonlimiting exemplary conversion of a cis-cleaving hammerhead ribozyme to a trans-cleaving hammerhead ribozyme is shown in FIGS. 17B and 17D, where loop II was removed to create the trans-cleaving hammerhead ribozyme of FIG. 17D. The substrate polynucleotide strand that is cleaved in FIGS. 17C and 17D is shown as a broken line.

In certain embodiments, removal of loop I or loop II to create a trans-cleaving hammerhead ribozyme from a cis-cleaving hammerhead ribozyme results in a large reduction in ribozyme activity. In certain embodiments, a bulge within the stem may compensate at least partially for the reduction in activity resulting from the loss of the loop at the end of the stem. Therefore, in certain embodiments, a cis-cleaving hammerhead ribozyme may be converted to a trans-cleaving hammerhead ribozyme by removing a loop and replacing it with a bulge within the stem from which the loop was removed.

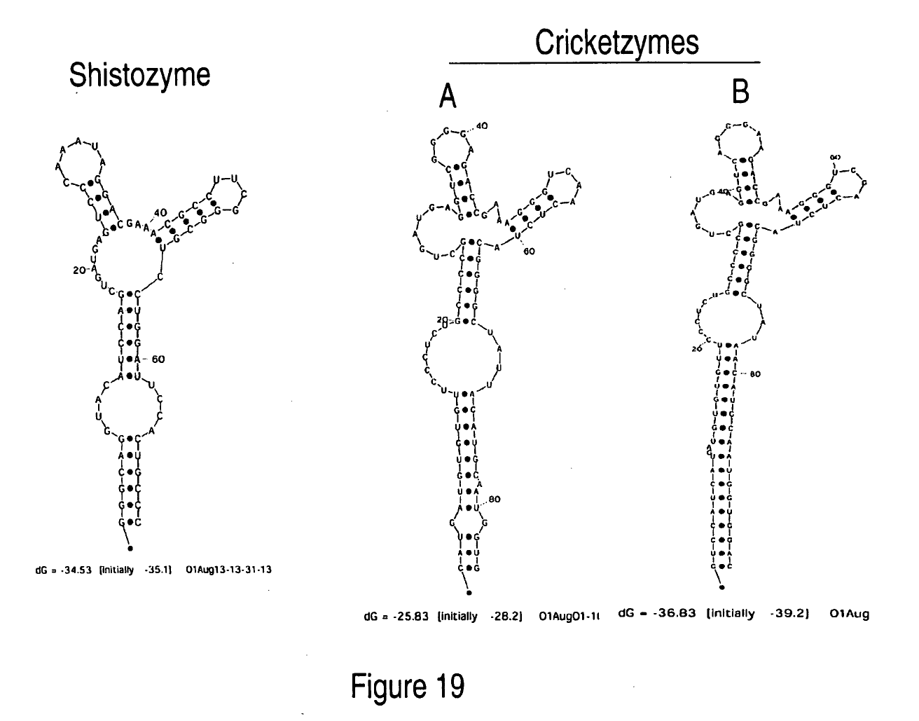

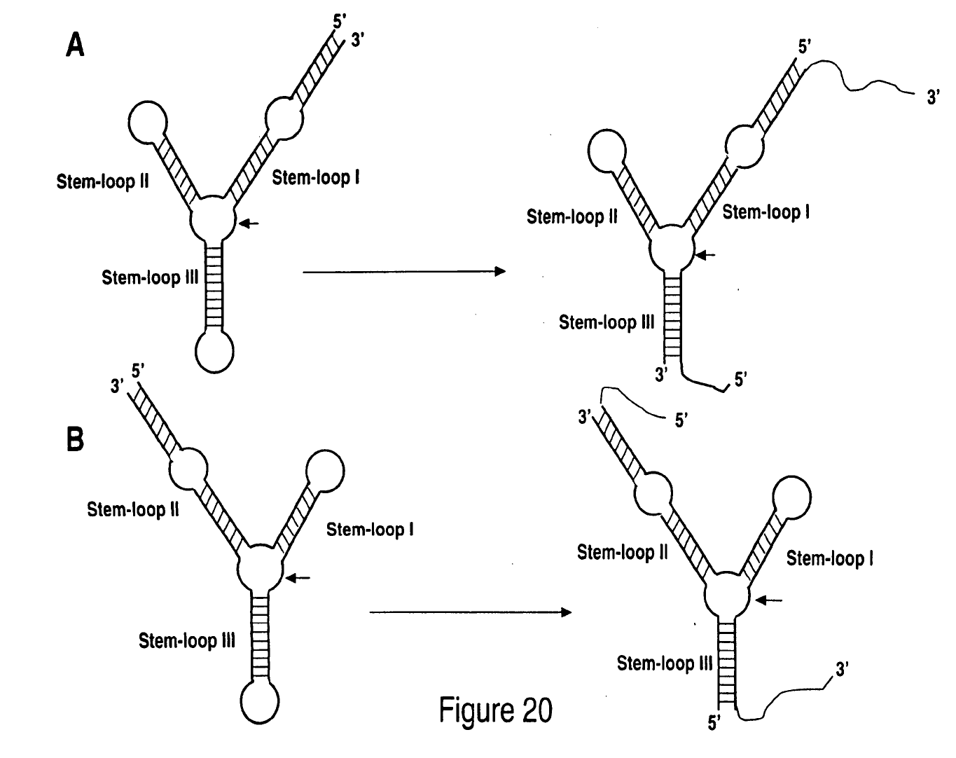

In certain embodiments, a natural I/I cis-cleaving hammerhead ribozyme may be converted to a I/III trans-cleaving hammerhead ribozyme by removing loop III. For example, Schistozyme is a I/I cis-cleaving hammerhead ribozyme that has a bulge within stem I. By removing loop III, Schistozyme can be converted into a I/III trans-cleaving hammerhead ribozyme. In certain embodiments, the removal of loop III has little or no effect on ribozyme activity. In certain embodiments, other I/I cis-cleaving hammerhead ribozymes, such as the Dolichopoda cave cricket ribozymes shown in FIG. 19, may also be converted to I/III trans-cleaving hammerhead ribozymes by removing loop III.

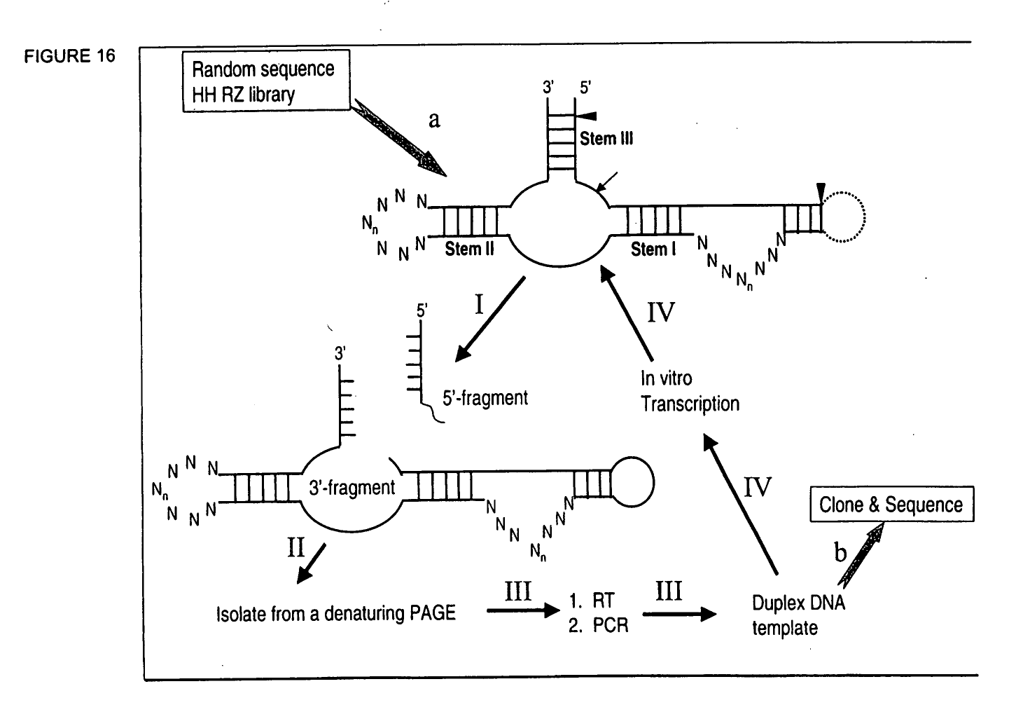

In certain embodiments, trans-cleaving hammerhead ribozymes may be selected in vitro. A nonlimiting exemplary selection scheme is shown in FIG. 16. In certain embodiments, trans-cleaving hammerhead ribozymes may be created by selecting a cis-cleaving hammerhead ribozyme. After selection of a cis-cleaving hammerhead ribozyme, a loop is removed to create the trans-cleaving hammerhead ribozyme. In certain embodiments, as shown in FIG. 16, a library of III/III cis-cleaving hammerhead ribozymes is made in which stem I contains a bulge. In certain embodiments, the bulge is an asymmetric bulge. In certain embodiments, following selection, loop I is removed to create a I/III trans-cleaving hammerhead ribozyme having a bulge within stem I.

In certain embodiments, a library of I/I cis-cleaving hammerhead ribozyme s is made in which stem I contains a bulge. In certain embodiments, the bulge is an asymmetric bulge. In certain embodiments, following selection, loop III is removed to create a I/III trans-cleaving hammerhead ribozyme having a bulge within stem I.

In certain embodiments, a library of ribozymes is made in which the bulge within stem I is randomized across the members of the library. In certain embodiments, a library of ribozymes is made in which loop II is randomized across the members of the library. In certain embodiments, a library of ribozymes is made in which both loop II and the bulge within stem I are randomized across the members of the library. In certain embodiments, for a library of III/III cis-cleaving hammerhead ribozymes, the core, stem I, loop I, stem II, and stem III are constant across the members of the library. In certain embodiments, for a library of I/I cis-cleaving hammerhead ribozymes, the core, stem I, stem II, stem III, and loop III are constant across the members of the library. In certain embodiments, either loop II or the bulge within stem I is also constant across the members of the library.

In certain embodiments, each randomized bulge is between 2 and 20 nucleotides long. In certain embodiments, each randomized bulge is between 3 and 10 nucleotides long. In certain embodiments, each randomized bulge is between 3 and 8 nucleotides long. In certain embodiments, each randomized bulge is 3, 4, 5, 6, 7, or 8 nucleotides long. In certain embodiments, each randomized loop is between 2 and 20 nucleotides long. In certain embodiments, each randomized loop is between 3 and 10 nucleotides long. In certain embodiments, each randomized loop is between 3 and 8 nucleotides long. In certain embodiments, each randomized loop is 3, 4, 5, 6, 7, or 8 nucleotides long.

In certain embodiments, a library of ribozymes is synthesized by in vitro transcription. In certain embodiments, a library of ribozymes is chemically synthesized.

In certain embodiments, the library of ribozymes is incubated under conditions allowing cleavage of the ribozymes and those ribozymes that have cleaved are separated from substantially all ribozymes that have not cleaved. In certain embodiments, incubation of the ribozyme library occurs at 37° C. in buffer comprising 50 mM Tris-HCl (pH 7.0) and 0.1 mM Mg²⁺. In certain embodiments, the ribozymes that have cleaved are separated from substantially all of the ribozymes that have not cleaved on the basis of size and/or charge. In certain embodiments, the separating step is denaturing polyacrylamide gel electrophoresis, or denaturing PAGE.

In certain embodiments, following separation of the ribozymes that cleaved from substantially all of the ribozymes that have not cleaved, the cleaved ribozymes are isolated, reverse transcribed, and amplified by PCR. In certain embodiments, one of the primers used for PCR adds back the sequence that was cleaved from the ribozyme, and one of the primers adds a promoter sequence for transcribing the PCR product into RNA. In certain embodiments, the promoter sequence is a promoter for RNA polymerase. In various embodiments, any suitable RNA polymerase promoter may be used, including, but not limited to, T7 RNA polymerase; SP6 RNA polymerase, and Qβ-replicase. In certain embodiments, following PCR amplification of the ribozymes, the PCR products are cloned into a vector, transformed into bacteria, and individual clones are isolated and sequenced.

In certain embodiments, the PCR-amplified ribozyme sequences are transcribed and the selection process is repeated. In certain embodiments, the selection process is repeated 1 to 20 times. In certain embodiments, the selection process is repeated 3 to 10 times. In certain embodiments, the selection process is repeated 5, 6, or 7 times.

In certain embodiments, following selection of a I/I cis-cleaving hammerhead ribozyme having a bulge within stem I, loop III is removed to create a I/III trans-cleaving hammerhead ribozyme. In certain embodiments, following selection of a III/III cis-cleaving hammerhead ribozyme having a bulge within stem I, loop I is removed to create a I/III trans-cleaving hammerhead ribozyme.

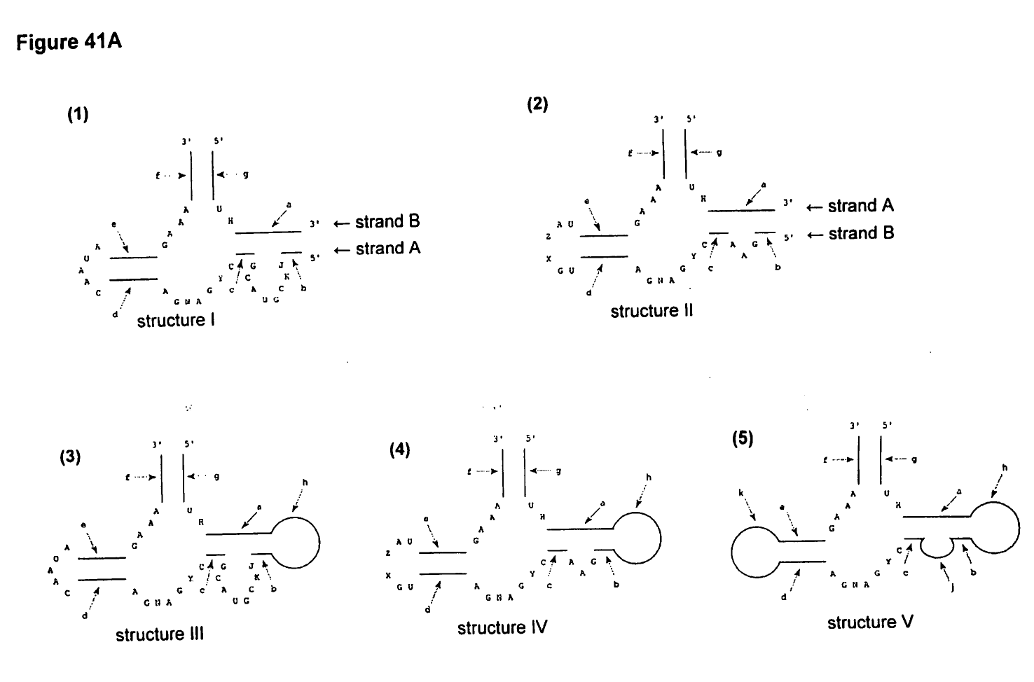

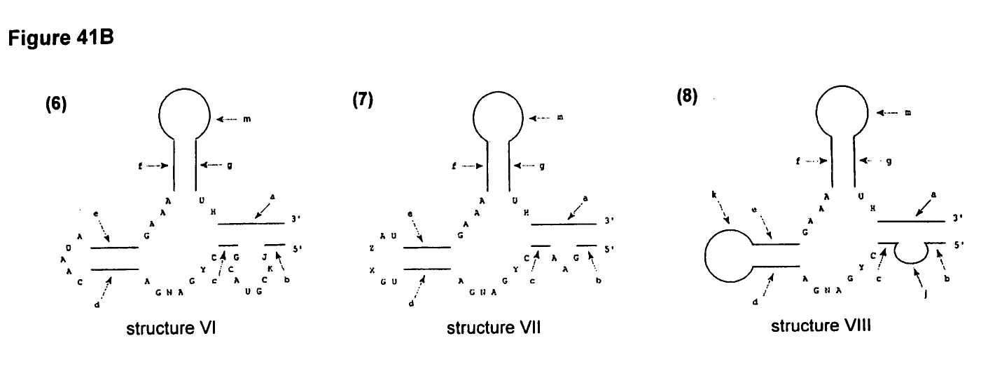

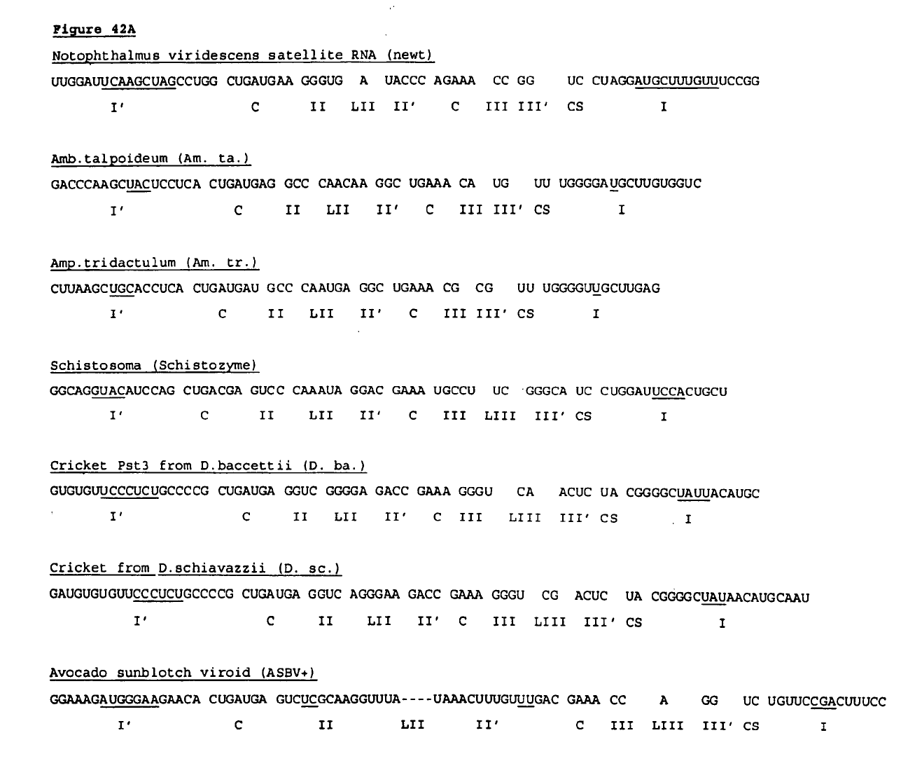

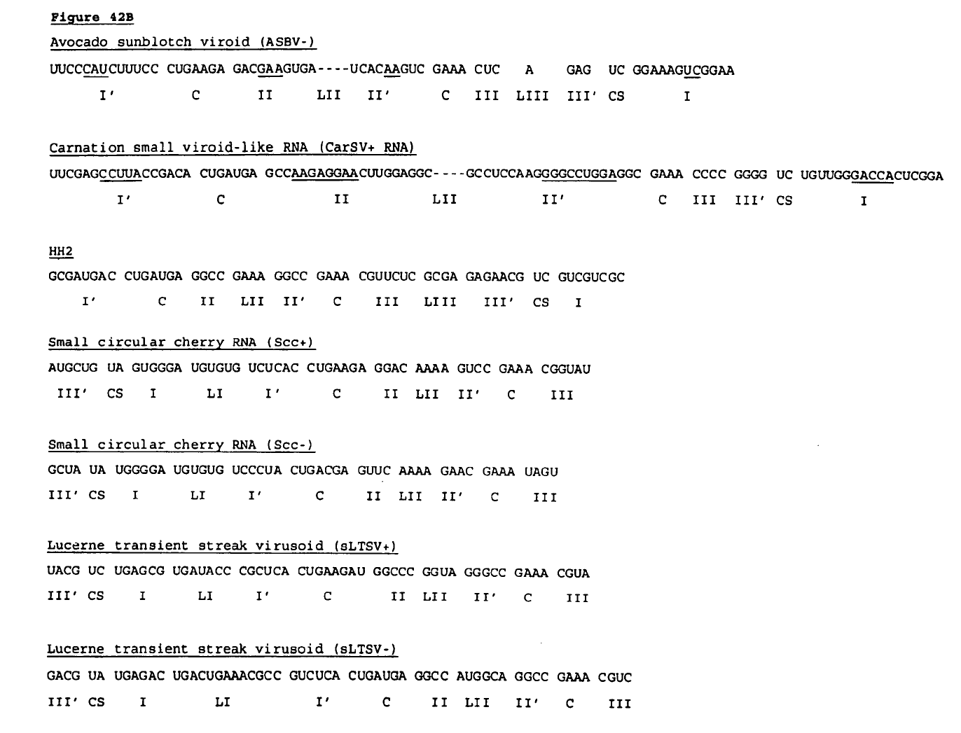

In certain embodiments, the invention provides a method of cleaving a target RNA. In certain embodiments, the target RNA is cleaved in vitro. In certain embodiments, the target RNA is cleaved in vivo. In certain embodiments, a trans-cleaving hammerhead ribozyme is selected, which cleaves the target RNA. In certain embodiments, a region of the target RNA that contains a 5′-UH-3′ dinucleotide is identified, where H is selected from A, C, and U. In certain embodiments, a III/III or III cis-cleaving hammerhead ribozymes is designed, wherein the portion of the strand of stem III that is 5′ of the cleavage site is identical to a portion of the target RNA that is 5′ of the UH dinucleotide and is immediately adjacent to that dinucleotide (see, e.g., FIG. 41A(5), g); and the portion of the strand of stem I that is 3′ of the cleavage site is identical to a portion of the target RNA that is 3′ of the UH dinucleotide and is immediately adjacent to that dinucleotide (see, e.g., FIG. 41A(5), a).

In certain embodiments, a portion of the other strand of stem III is complementary to the portion of the target RNA that is 5′ of the UH dinucleotide (see, e.g., FIG. 41A(5), f), and a portion of the other strand of stem I is complementary to the portion of the target RNA that is 3′ of the UH dinucleotide (see, e.g., FIG. 41A(5), b and c). The other strand of stem I is interrupted by a bulge such that the bulge is flanked on each side by at least two nucleotides that are complementary to the target RNA (see, e.g., FIG. 41A(5), j).

In certain embodiments, the bulge within stem I is constant and loop 2 is random across the members of the library. In certain embodiments, loop 2 is constant and the bulge within stem I is random across the members of the library. In certain embodiments, both loop II and the bulge within stem I are random across the members of the library.

In certain embodiments, selection is carried out substantially as described above. In certain embodiments, following selection, a III/III cis-cleaving hammerhead ribozyme may be converted to a I/III trans-cleaving hammerhead ribozyme by removing loop I. In certain embodiments, following selection, a I/I cis-cleaving hammerhead ribozyme may be converted to a I/III trans-cleaving hammerhead ribozyme by removing loop III. In certain embodiments, a trans-cleaving hammerhead ribozyme is made, which comprises one strand of stem I, including the bulge within stem I, a portion of the core, stem II and loop II, a second portion of the core, and one strand of stem III (see, e.g., FIG. 41A(5); the ribozyme comprises b, j, c, CYGANGA, d, k, e, GAAA, and f).

In certain embodiments, rather than selecting a ribozyme with certain sequences in loop II and in the bulge within stem I from a library, such sequences are chosen from a known hammerhead ribozyme. In certain embodiments, a trans-cleaving hammerhead ribozyme may be altered by adapting the stem I and stem III sequences according to the chosen target RNA, as described above, where a 5′-UH-3′ dinucleotide within the target RNA is chosen and certain portions of the ribozyme are chosen to be identical or complementary to target RNA sequences surrounding the 5′-UH-3′ dinucleotide. In certain embodiments, such adaptation has little or no effect on ribozyme activity.

In certain embodiments, a ribozyme is chemically modified. In certain embodiments, one or more nucleotides within a stem, loop, or bulge is chemically modified. In certain embodiments, at least one chemical modification is made to a base, ribose, or phosphate group. In certain embodiments, chemical modification results in a ribozyme that is more active than the ribozyme without the chemical modification. In certain embodiments, at least one nucleotide within at least one of stem I, a bulge within stem I, or loop I is chemically modified. In certain embodiments, at least one nucleotide within at least one of stem II, a bulge within stem II, or loop II is chemically modified. In certain embodiments, at least one nucleotide within at least one of stem I, a bulge within stem I, or loop I is chemically modified, and at least one nucleotide within at least one of stem II, a bulge within stem II, or loop II is also chemically modified. In certain embodiments, chemical modifications are introduced during chemical synthesis of a ribozyme using modified nucleosides. Certain chemically modified nucleosides are available, e.g., from Proligo.

In certain embodiments, a nucleotide within stem I is chemically modified. In certain embodiments, a nucleotide within stem II is chemically modified. Exemplary modifications include, but are not limited to, groups having an aldehyde, an amino, a thiol, and a malimide. In certain embodiments, a nucleotide within stem I is chemically modified to have an aldehyde, and a nucleotide within stem II is chemically modified to have an amine. In certain embodiments, a nucleotide within stem I is chemically modified to have an amine, and a nucleotide within stem II is chemically modified to have an aldehyde. In certain embodiments, a nucleotide within stem I is chemically modified to have a thiol, and a nucleotide within stem II is chemically modified to have a malimide. In certain embodiments, a nucleotide within stem I is chemically modified to have a malimide, and a nucleotide within stem II is chemically modified to have a thiol. In certain embodiments, a nucleotide within stem I is chemically modified to have a thiol, and a nucleotide within stem II is chemically modified to have a thiol.

In certain embodiments, when there is more than one chemically modified nucleotide in a ribozyme, at least two of the chemically modified nucleotides interact either covalently or noncovalently. In certain embodiments, at least two of the chemically modified nucleotides are capable of forming a disulfide bond. In certain embodiments, at least two of the chemically modified nucleotides are capable of forming an amide bond. In certain embodiments, at least two of the chemically modified nucleotides interact electrostatically. Exemplary chemical modifications are described, e.g., in U.S. Pat. Nos. 6,159,951; 6,379,954; 6,093,555; and in Gayret al. (1997) “Chemical and enzymatic approaches to construct modified RNAs” in Ribozyme Protocols, Humana Press.

In certain embodiments, a polynucleotide encoding a trans-cleaving hammerhead ribozyme is made, e.g., by reverse transcription or chemical synthesis. In certain embodiments, the polynucleotide is cloned into a vector. In certain embodiments, the vector also comprises sequences for expression of the ribozyme in vivo. Such sequences are known in the art and may include, but are not limited to, promoters, terminators, enhancers, polyadenylation signals, introns, etc. In certain embodiments, the vector is introduced into a host cell.

In certain embodiments, a target RNA is cleaved in vivo by introducing into a cell a trans-cleaving hammerhead ribozyme that cleaves the target RNA into a cell. In various embodiments, introduction of a ribozyme or a polynucleotide encoding a ribozyme into the cell is accomplished by methods known in the art, e.g., transforming, transfecting, infecting, transducing, introduction by passive uptake, or conjugation-induced introduction. In certain embodiments, introduction may also be accomplished, e.g., by injecting a cell with a ribozyme, which has been made by a method known in the art, such as in vitro transcription or chemical synthesis, and/or with a polynucleotide encoding a ribozyme. Introduction of the ribozyme may also occur by infecting the cell with an RNA virus carrying the ribozyme within its genome.

In certain embodiments, once introduced, the ribozyme cleaves a target RNA in vivo. In certain embodiments, following introduction of the ribozyme, levels of the target RNA are reduced in vivo. In certain embodiments, if the target RNA encodes a protein, levels of the protein may also be reduced following introduction of the ribozyme.

In certain embodiments, the invention provides methods for reducing RNA levels in an animal. Suitable animals include, but are not limited to, humans; non-human primates, including monkeys and chimpanzees; rodents, including mice, hamsters, and rats; livestock animals, including goats, sheep, chickens, pigs, and cows; and other animals, including rabbits, guinea pigs, cats, dogs, horses, etc. In certain embodiments, the invention provides methods for reducing RNA levels in other organisms, such as bacteria, yeast, insects, fish, and plants.

In certain embodiments, a ribozyme or a polynucleotide encoding a ribozyme is introduced into an organism. In certain embodiments, a pharmaceutical compositions comprising a ribozyme, together with a pharmaceutically acceptable diluent, carrier, solubilizer, emulsifier, preservative and/or adjuvant is provided.

In certain embodiments, acceptable formulation materials preferably are nontoxic to recipients at the dosages and concentrations employed.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In various embodiments, suitable formulation materials include, but are not limited to, amino acids (such as, but not limited to, glycine, glutamine, asparagine, arginine or lysine); antimicrobials; antioxidants (such as, but not limited to, ascorbic acid, sodium sulfite or sodium hydrogen-sulfite); buffers (such as, but not limited to, borate, bicarbonate, Tris-HCl, citrates, phosphates or other organic acids); bulking agents (such as, but not limited to, mannitol or alycine); chelating agents (such as, but not limited to, ethylenediamine tetraacetic acid (EDTA)); complexing agents (such as, but not limited to, caffeine, polyvinylpyrrolidone, beta-cyclodextrin or hydroxypropyl-beta-cyclodextrin); fillers; monosaccharides; disaccharides; and other carbohydrates (such as, but not limited to, glucose, mannose or dextrins); proteins (such as, but not limited to, serum albumin, gelatin or immunoglobulins); coloring, flavoring and diluting agents; emulsifying agents; hydrophilic polymers (such as, but not limited to, polyvinylpyrrolidone); low molecular weight polypeptides; salt-forming counterions (such as, but not limited to, sodium); preservatives (such as, but not limited to, benzalkonium chloride, benzoic acid, salicylic acid, thimerosal, phenethyl alcohol, methylparaben, propylparaben, chlorhexidine, sorbic acid or hydrogen peroxide); solvents (such as, but not limited to, glycerin, propylene glycol or polyethylene glycol); sugar alcohols (such as, but not limited to, mannitol or sorbitol); suspending agents; surfactants or wetting agents (such as, but not limited to, pluronics, PEG, sorbitan esters, polysorbates such as polysorbate 20, polysorbate 80, triton, tromethamine, lecithin, cholesterol, tyloxapal); stability enhancing agents (such as, but not limited to, sucrose or sorbitol); tonicity enhancing agents (such as, but not limited to, alkali metal halides, preferably sodium or potassium chloride, mannitol sorbitol); delivery vehicles; diluents; excipients and/or pharmaceutical adjuvants. (see, e.g., Remington's Pharmaceutical Sciences, 18^{th} Edition, A. R. Gennaro, ed., Mack Publishing Company (1990).

In certain embodiments, an optimal pharmaceutical composition may be determined by one skilled in the art depending upon, for example, the intended route of administration, delivery format and desired dosage. See, for example, Remington's Pharmaceutical Sciences, supra.

In certain embodiments, a primary vehicle or carrier in a pharmaceutical composition may be either aqueous or non-aqueous in nature. For example, in certain embodiments, a suitable vehicle or carrier may be water for injection, physiological saline solution or artificial cerebrospinal fluid, possibly supplemented with other materials common in compositions for parenteral administration. In certain embodiments, neutral buffered saline or saline mixed with serum albumin are further nonlimiting exemplary vehicles.

In certain embodiments, a ribozyme is administered to a subject in an amount of about 0.1 mg/kg to about 1 g/kg. In certain embodiments, a ribozyme is administered to a subject in an amount of about 1 mg/kg to about 200 mg/kg. In certain embodiments, a ribozyme is administered to a subject in an amount of about 5 mg/kg to about 100 mg/kg. In certain embodiments, a ribozyme is administered to a subject in an amount of about 10 mg/kg to about 60 mg/kg.

In certain embodiments, a ribozyme is used for the therapeutic treatment of an animal. In certain embodiments, a ribozyme is used for diagnostic or other non-therapeutic purposes.

### EXAMPLES

The following examples, including the experiments conducted and results achieved are provided for illustrative purposes only and are not to be construed as limiting the present invention in any way.

### Example 1

### Chimeric Hammerhead Ribozymes

Three different naturally occurring ribozymes belonging to the hammerhead family, tobacco ringspot virus (sTRSV), lucerne transient streak virus (vLTSV), and peach latent mosaic viroid (PLMVd), were used to study certain chimeric hammerhead ribozymes. Each of these ribozymes has the same core motif while their stems and loops are different.

To test the effects of changes to loops I and II on the activity of those hammerhead ribozymes, loop I, loop II, or both loop I and loop II of sTRSV were swapped with the corresponding loops from either vLTSV− or PLMVd. The resulting chimeric ribozymes were designated sTRSV+vLT−1 (sTRSV containing loop I from vLTSV), sTRSV+vLT−2 (sTRSV containing loop II from vLTSV), sTRSV+vLT−1&2 (sTRSV containing both loop I and loop II from vLTSV), sTRSV+PL−1 (sTRSV containing loop I from PLMVd), sTRSV+PL−2 (sTRSV containing loop II from PLMVd), and sTRSV+PL−1 & 2 (sTRSV containing both loop I and loop II from PLMVd). Those chimeric hammerhead ribozymes are depicted schematically in FIGS. 2 and 3.

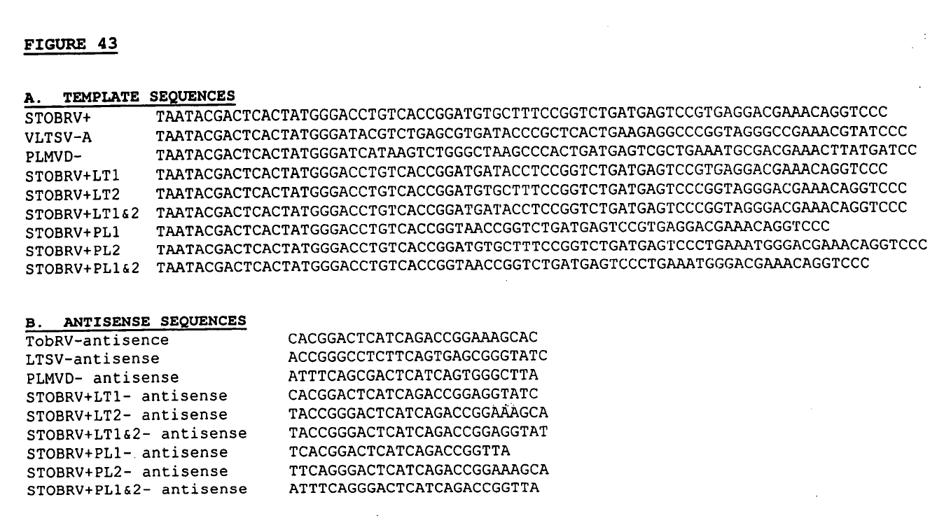

All of the wild-type and chimeric ribozymes were transcribed in vitro using a DNA template carrying a T7 RNA polymerase promoter. The template sequences are shown in FIG. 43A. In vitro transcription reactions contained antisense DNA oligonucleotides (oligos) to prevent ribozyme cleavage during transcription. The antisense sequences are shown in FIG. 43B. Generally, the antisense oligos hybridized to a portion of stem I, a portion of stem II, and the portion of the core that connects stem I and stem II. A separate antisense oligonucleotide was designed for each ribozyme.

Fifty-microliter in vitro transcription reactions were carried out at 37° C. and comprised 80 mM HEPES (pH 7.5), 20 mM MgCl₂, 40 mM DTT, 2 mM Spermidine, 4 mM each NTP (ATP, CTP, GTP, and UTP), 1.5 μl α-[³²P]UTP (3000 Ci/mMol; NEN), 400 units of T7 RNA polymerase (Epicentre Technologies), 10-50 pmol of DNA template, and 40 μM of an antisense oligonucleotide. Separate transcription reactions were carried out for each template. Reactions were incubated for 1-2 hours, followed by DNAse I (10 units, Promega Corporation) digestion for 15 minutes at 37° C. to remove the DNA template. EDTA was then added to a final concentration of 30 mM. The reaction was passed through a P-30 gel filtration spin column (Bio-Rad) to remove unincorporated nucleotides from the RNA transcripts. The RNA was then precipitated with ethanol. The precipitated RNA was pelleted by centrifugation and resuspended in loading buffer, which included 7 M urea and 5 mM EDTA, and then purified by denaturing polyacrylamide gel electrophoresis (PAGE).

The initial rate of cleavage, or k_{obs}, for each of the wild-type and chimeric ribozymes was measured as the percentage cleaved as a function of time using the method described in McCaffrey et al. (1999) in Intracellular Ribozyme Applications: Principles and Protocols, Horizon Scientific Press, pp. 57-68, which is incorporated herein by reference for any purpose.

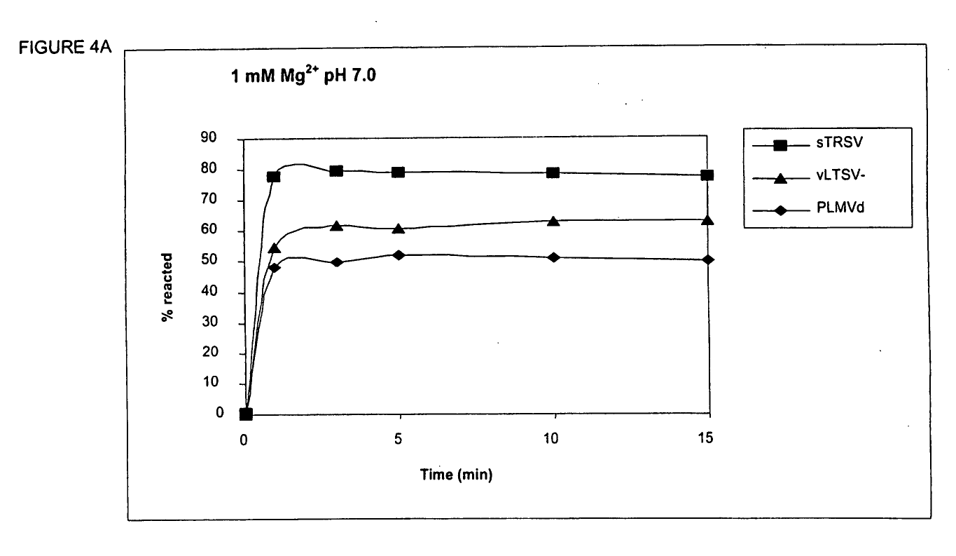

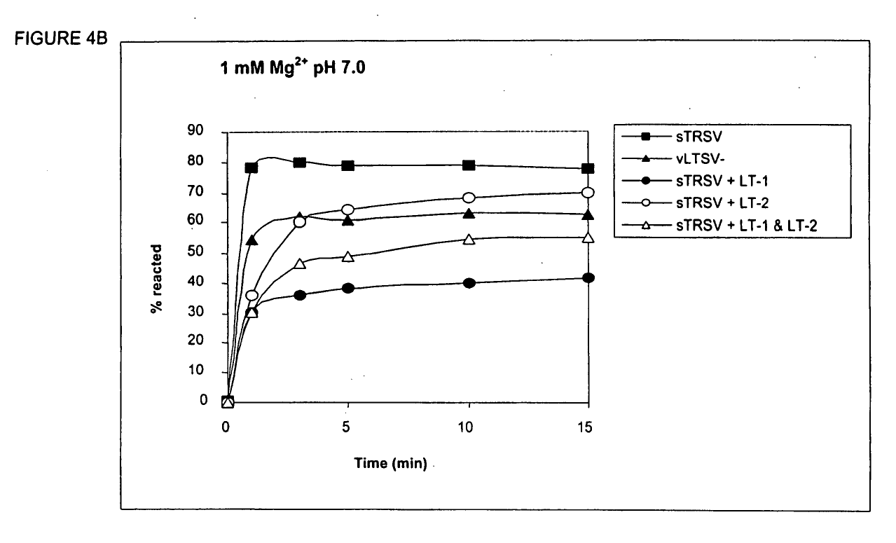

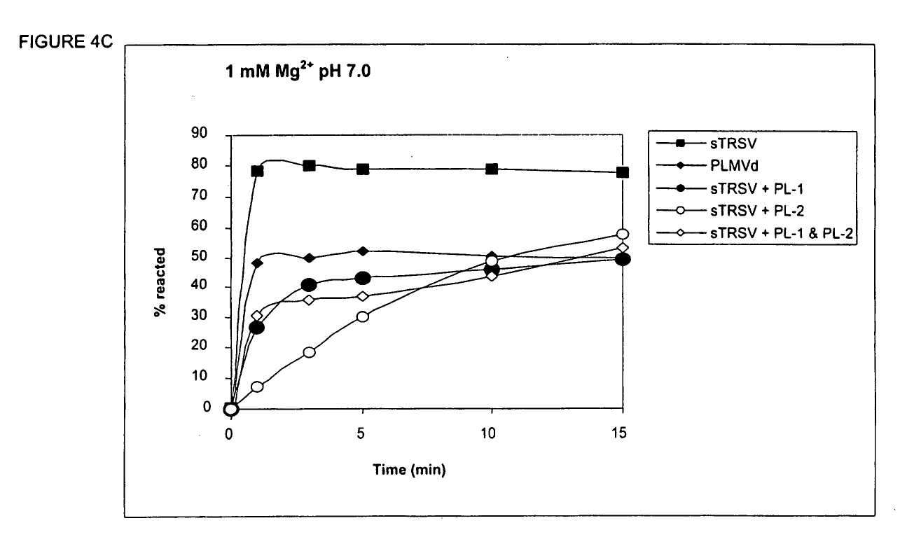

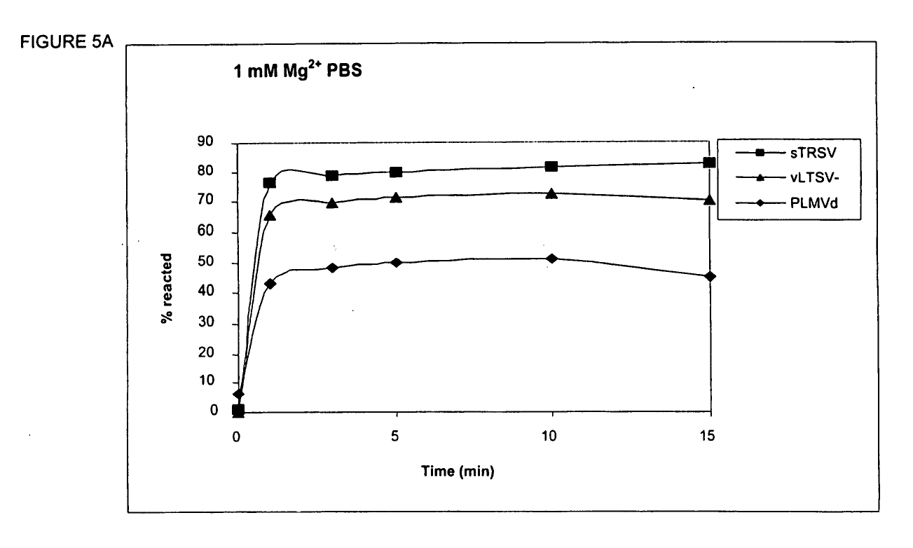

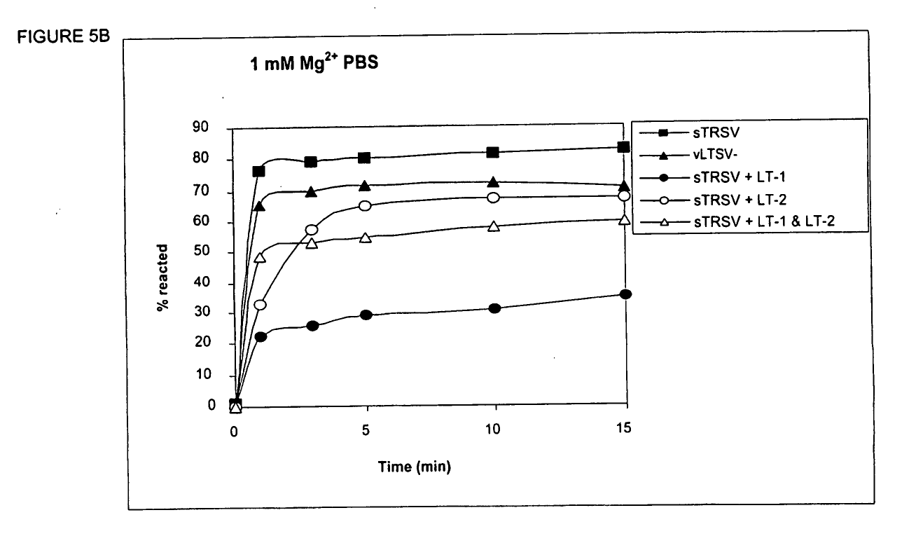

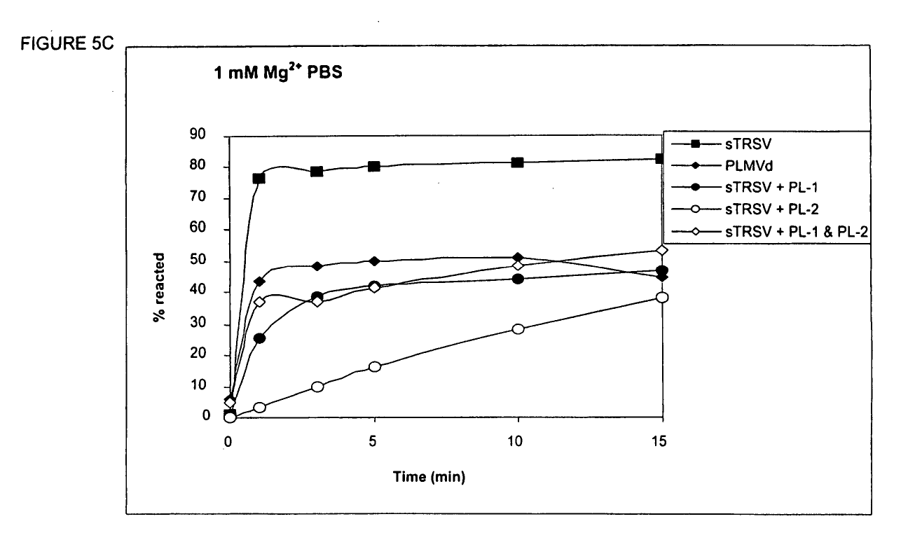

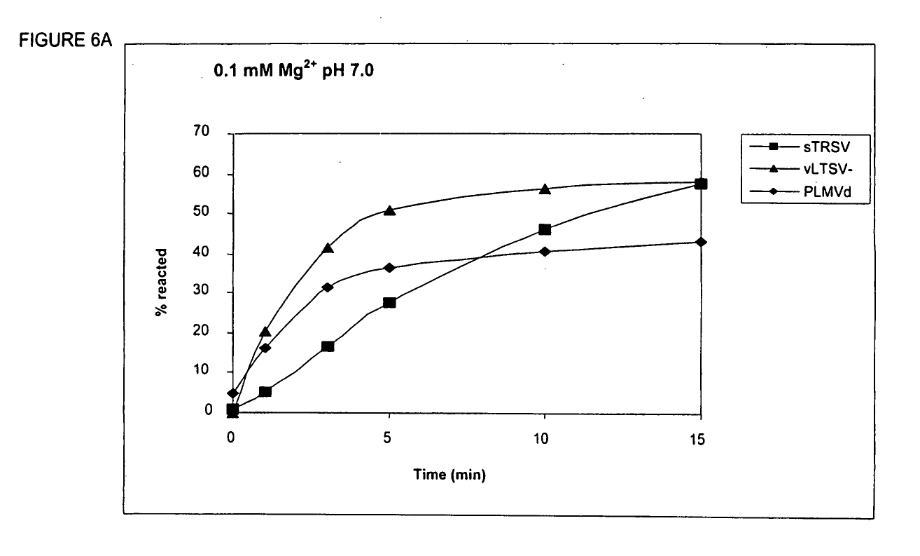

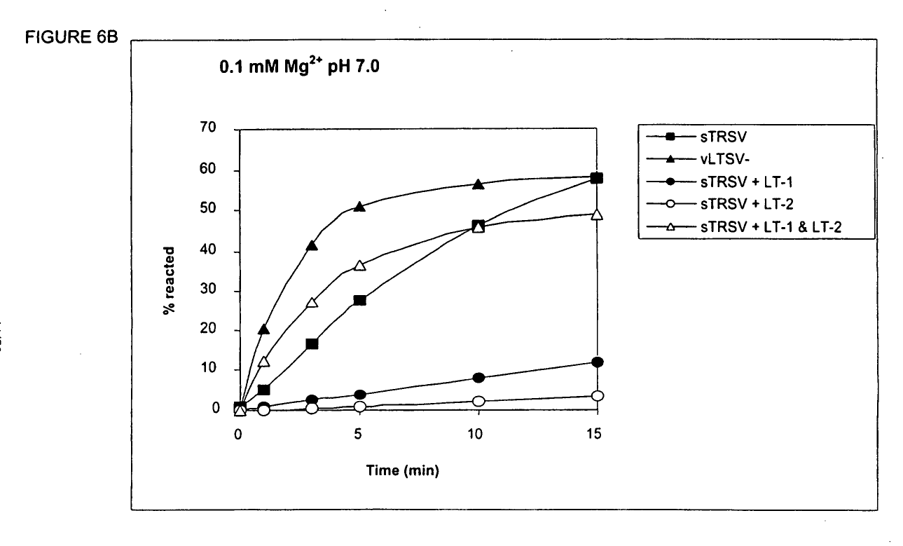

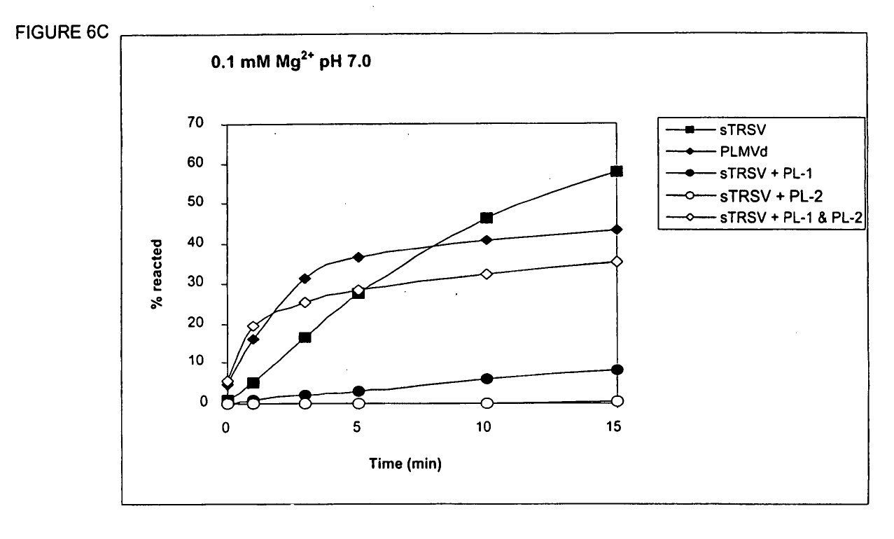

Three different conditions were used to measure the rate of cleavage: Tris-HCl (pH 7.0), 1 mM MgCl₂ (FIG. 4); PBS (pH 7.5), 1 mM MgCl₂ (FIG. 5); and Tris-HCl (pH 7.0), 0.1 mM MgCl₂ (FIG. 6). Ribozymes were first dissolved in 0.0002 mM EDTA and preheated to desired temperature. Cleavage reactions were started by adding an appropriate volume of the desired 2× buffer so that the final buffer composition in the reaction was 1×.

Any differences between the activities of each wild-type ribozyme in Tris buffer versus in PBS were not great (see FIGS. 4A and 5A). Furthermore, any differences between the activities of sTRSV, vLTSV, and PLMVd in either buffer at 1 mM MgCl₂ were not great (FIGS. 4A and 5A).

Substituting either loop I or loop II of sTRSV with the corresponding loop from vLTSV− or PLMVd resulted in a decrease in activity at 1 mM Mg²⁺ (see FIG. 4, B and C; and FIG. 5, B and C). Substituting loop I of sTRSV with loop I of vLTSV− had a greater effect than substituting loop II-of sTRSV-with loop II of vLTSV− (see FIGS. 4B and 5B). Substitution of both loop I and loop II of sTRSV with loops I and II from vLTSV− resulted in a small decrease in activity as compared to sTRSV or vLTSV−. In contrast, substituting loop II of sTRSV with loop II of PLMVd had a greater effect on the activity of sTRSV than substituting loop I of sTRSV with loop I of PLMVd (see FIGS. 4C and 5C). Substituting both loop I and loop II of sTRSV with loop I and loop II of PLMVd resulted in a small decrease in activity as compared to sTRSV or vLTSV−.

The effect of loop substitutions is more profound when measured at 0.1 mM Mg²⁺ (FIG. 6 and Table 1). The percentage of ribozymes cleaved as a function of time was measured in Tris-HCl (pH 7.0) and 0.1 mM Mg²⁺. When either loop I or loop II of sTRSV is individually th loop from vLTSV− or PLMVd, the rate of cleavage decreased (FIG. 6, B and C). The initial rate of cleavage (K_{obs}) decreased by up to 300 fold when either loop of sTRSV was substituted with a loop from vLTSV− or PLMVd. Substitution of both loop I and loop II of sTRSV with loop I and loop II from either vLTSV− or PLMVd resulted in near wild-type activity (FIG. 6, B and C).

TABLE 1
Wild-type and chimeric ribozyme kinetics at 0.1 mM Mg²⁺

| ribozyme | k_{obs} (min⁻¹) | fold loss of activity |
|---|---|---|
| sTRSV | 1.2 | 1 |
| vLTSV | 1.4 | 1 |
| PLMVd | 7.1 × 10⁻¹ | 2 |
| sTRSV + LT-1 | 1.6 × 10⁻¹ | 7 |
| sTRSV + LT-2 | 4.5 × 10⁻² | 26 |
| sTRSV + LT-1 & LT-2 | 1.0 | 1 |
| sTRSV + PL-1 | 1.1 × 10⁻¹ | 11 |
| sTRSV + PL-2 | 3.9 × 10⁻³ | 309 |
| sTRSV + PL-1 & PL-2 | 4.4 × 10⁻¹ | 3 |

These results suggest that the sequence of loops I and II are important for hammerhead ribozyme activity, especially at low Mg²⁺ concentrations. One possible explanation for that observation is that loop I and loop II interact with one another for optimal activity. This explanation may explain why substitution of both loops of sTRSV with both loops from either vLTSV− or PLMVd results in a chimeric ribozyme with greater activity than a chimeric ribozyme with only one loop substituted. This possible explanation, however, is in no way limiting, and such interactions are not required.

### Example 2

### Effect of Mg²⁺ Concentration on the Activity of Chimeric Hammerhead Ribozymes

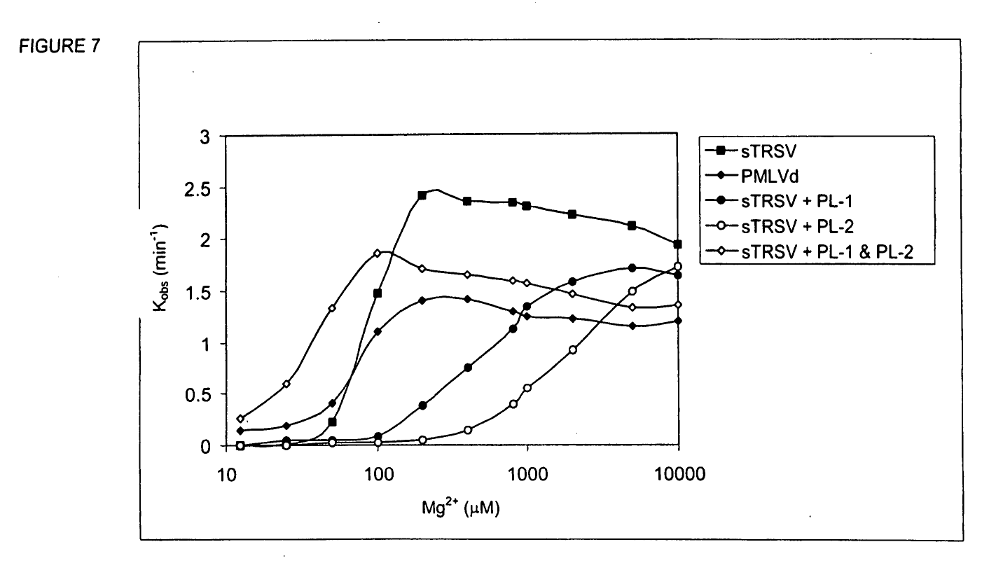

The effect of Mg²⁺ concentration on hammerhead ribozyme activity was determined by measuring the rate of cleavage for sTRSV and PLMVd wild-type and chimeric ribozymes at Mg²⁺ concentrations ranging from 0.01 mM to 10 mM (FIG. 7). The cleavage rate of both sTRSV and PLMVd reached half-saturation at approximately 0.05 mM Mg²⁺. Substitution of either loop I or loop II of sTRSV with the corresponding loop of PLMVd resulted in an increase of the apparent K_{eff} for Mg²⁺ of 1 to 2 orders of magnitude (Table 2). K_{eff} is the effective concentration of Mg²⁺ at the half maximal activity of the ribozyme.

TABLE 2
K_{eff} for Mg²⁺ of wild-type and chimeric ribozymes

| ribozyme | K_{eff} for Mg²⁺ (mM) |
|---|---|
| sTRSV | 0.07 |
| PLMVd | 0.062 |
| sTRSV + PL-1 | 0.43 |
| sTRSV + PL-2 | 8 |
| sTRSV + PL-1 & PL-2 | 0.03 |

sTRSV−PL1 reached half saturation at 0.43 mM Mg²⁺, while sTRSV−PL2 reached half saturation at approximately 8 mM Mg²⁺. As observed previously, substitution of loop II of sTRSV with loop II of PLMVd had a more profound effect than substitution of loop I of sTRSV with loop I of PLMVd, especially at 0.1 mM Mg²⁺ (see FIG. 6C). At higher Mg²⁺ concentration, however, substitution of only one loop of sTRSV was more readily tolerated by the ribozyme (see, e.g., FIGS. 4C and 5C). Thus, the deleterious effect of substituting only one loop on the cleavage rate of the ribozyme appears to be reduced by increasing the Mg²⁺ concentration.

A possible explanation for that observation is that interactions between nucleotides of loops I and II create a high affinity Mg²⁺ binding site that contributes to faster ribozyme cleavage. Another possible explanation is that interactions between loop I and loop II of a ribozyme may result in a lower energy transition state so that the ribozyme has greater activity at lower Mg²⁺ concentrations that ribozymes that lack such interactions. These possible explanations, however, are in no way limiting, and such Mg²⁺ binding sites and loop I/loop II interactions are not required.

Finally, substitution of both loops I and II of sTRSV with the corresponding loops of PLMVd resulted in a K_{eff} for Mg+² that was even lower than the K_{eff} of either wild-type sTRSV or wild-type PLMVd (0.03 mMA versus 0.07 mM and 0.062 mM, respectively). As discussed above, a possible explanation for these results is that tertiary interactions that contribute to the rate of cleavage may be mediated by the nucleotide sequences of loops I and II of the ribozyme. This possible explanation, however, is in no way limiting, and such interactions are not required.

### Example 3

### In Vivo Activity of Chimeric Hammerhead Ribozymes

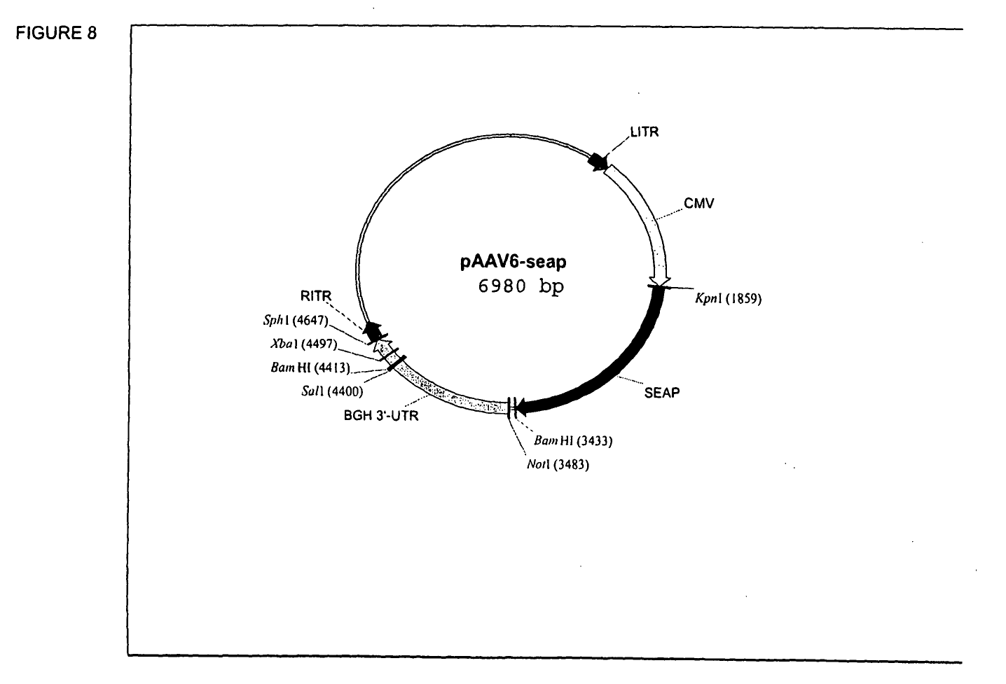

To demonstrate that the hammerhead ribozymes that cleaved at 0.1 mM MgCl₂ are functional in vivo, several wild-type and chimeric hammerhead ribozymes were cloned into a bovine growth hormone 3′-untranslated region (BGH 3′-UTR) downstream of a secreted alkaline phosphatase (SEAP) gene. The SEAP gene and the BGH 3′-UTR were located downstream of a cytomegalovirus (CMV) promoter in an adeno-associated viral vector (pAAV-6-seap; FIG. 8).

pAAV-6-seap was constructed by amplifying the seap gene (Promega) by PCR and cloning it into pCL (Promega) as a Kpn1/BamH1 fragment. The BGH 3′-UTR from pcDNA3.1 was cloned downstream of the seap gene. Additional restriction sites, SalI and XbaI, were introduced into the 3′-UTR by PCR cloning.

cDNA encoding each of the following hammerhead ribozymes were cloned into the XbaI and SalI sites of the pAAV-6-seap vector, within the BGH 3′ UTR: Schistosoma mansoni hammerhead ribozyme (Schistozyme; a naturally-occurring hammerhead ribozyme), PLMVd, vLTSV, sTRSV, sTRSV+PL−1 & PL−2, sTRSV+PL−1, and sTRSV+PL−2. Each recombinant vector was then purified and analyzed by restriction digest with SalI and XbaI for the presence of the ribozyme insert. The insert identities were further confirmed by sequencing.

HEK 293 cells (ATCC No. CRL 1573) plated in 96-well microtiter plates were used to test each construct. The cells were grown to 90% confluence in Dulbecco's Modified Eagle Medium (DMEM), supplemented with 2 mM Glutamine and 10% fetal bovine serum (FBS) and maintained at 37° C. with 5% CO₂. Fifty nanograms per well of each pAAV-6-seap construct containing an inserted ribozyme was transfected into HEK 293 cells using Lipofectamine 2000 (Invitrogen), according to the instructions provided with the product. Twenty-four hours after transfection, 15 μL of medium from each well was transferred to a white opaque 96-well flat bottom microtiter plate (Costar), and SEAP activity was measured using a chemiluminescent SEAP assay (Great EscAPe SEAP assay kit, Clontech) according to the manufacturer's instructions.

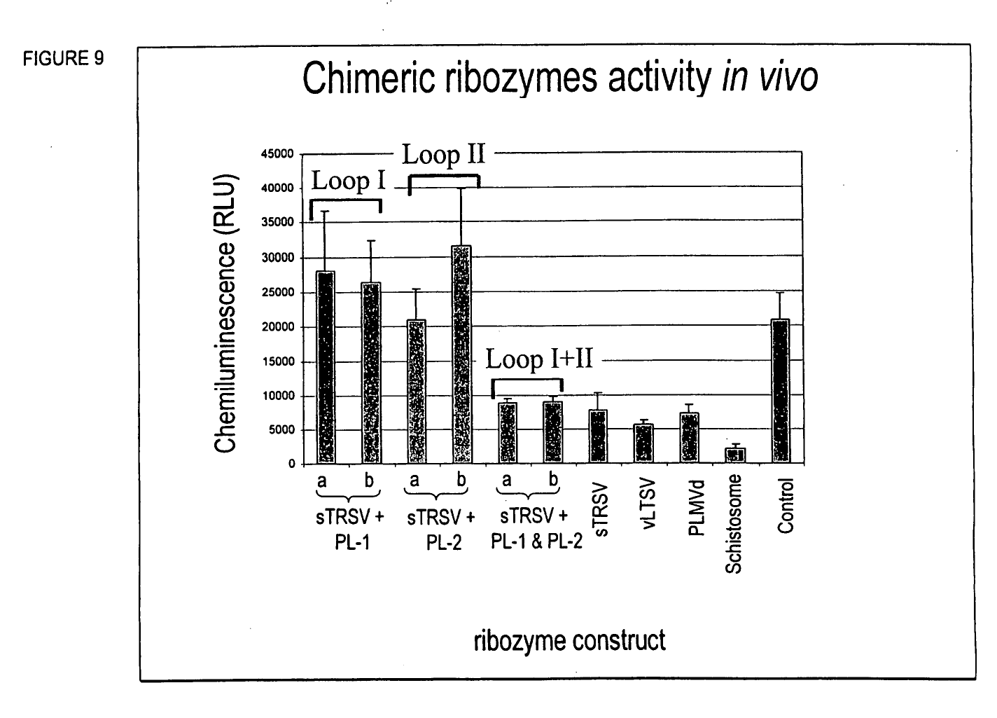

Results of in vivo activity of the wild-type and chimeric hammerhead ribozymes tested are shown in FIG. 9. The SEAP gene and BGH 3′-UTR (with inserted ribozyme) are transcribed from the CMV promoter to produce an mRNA containing the SEAP mRNA and the BGH 3′-UTR, which contains the inserted ribozyme, followed by the poly A tail. If the ribozyme cis-cleaves, then the poly A tail is cleaved from the mRNA and the mRNA will be degraded. In that case, the SEAP protein will not be expressed and the amount of chemiluminescence detected in the chemiluminescent SEAP assay will be lower than that observed from the control transfection, which uses the pAAV-6-seap construct without an inserted ribozyme.

All three naturally occurring hammerhead ribozymes, PLMVd, vLTSV and sTRSV, were active in vivo. As a result, lower levels of chemiluminescence were observed with each of those ribozymes inserted into the BGH 3′-UTR than was observed with the control. Schistozyme was also active in vivo. In contrast, recombinant plasmids carrying sTRSV+PL−1 or sTRSV+PL−2, which have only one loop of sTRSV replaced with a loop from PLMVd resulted in no substantial decrease in chemiluminescence from the of sTRSV were replaced with loops I and II from PLMVd, the resulting chimeric ribozyme was active in vivo.

The ribozymes that did not cleave efficiently in 0.1 mM Mg²⁺, sTRSV+PL−1 and sTRSV+PL−2, failed to cleave in vivo in this experiment. Those that were active in 0.1 mM Mg²⁺, including the wild-type ribozymes and chimeric sTRSV+PL−1 & PL−2, were active in vivo in this experiment. These results suggest that, in certain embodiments, the in vitro activity of a hammerhead ribozyme at low Mg⁺² (such as 0.1 mM) can serve as a predictor of the in vivo activity of the ribozyme.

### Example 4

### sTRSV Mutants

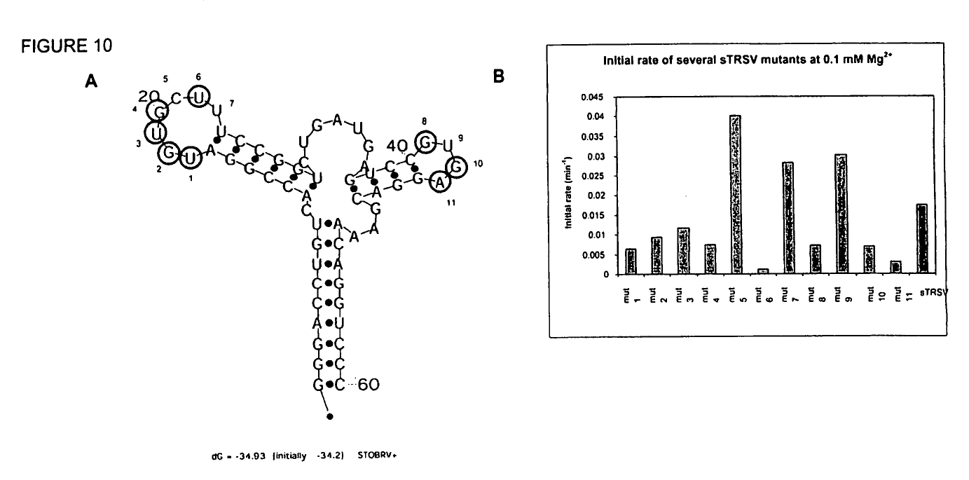

To observe the impact of changing the nucleotides within loops I and II of sTRSV on activity at low Mg⁺² concentrations, all 11 nucleotides in both loops of sTRSV were substituted with cytosine, with the exception of the cytosine shown as nucleotide 5, which was substituted with guanosine (FIG. 10A). The cleavage rate of wild type sTRSV and each of the mutants is shown in FIG. 10B. Mutation at seven of the eleven positions showed a deleterious effect on the cleavage rate of the ribozyme in 50 mM Tris-HCl (pH 7.0) and 0.1 mM MgCl₂. Mutation at positions 6 and 11 appeared to have the strongest effect on the cleavage rate.

One possible explanation for those results is that there is an interactions between the nucleotides in loop I and loop II of sTRSV and mutation of one of those nucleotides may disrupts that interaction and cause a reduction in the cleavage rate of the ribozyme at low Mg²⁺ concentrations. This possible explanation, however, is in no way limiting, and such interactions are not required.

Substitution of certain nucleotides in loop I and loop 2, specifically at positions 5, 7, and 9, enhanced the rate of cleavage beyond that of the wild type ribozyme. According to certain embodiments, one may be able to design certain hammerhead ribozymes with advantageous cleavage rates using the in vitro selection methods depicted, e.g., in FIGS. 14-16.

### Example 5

### Schistozyme Mutants

Schistozyme is a naturally occurring ribozyme that exists in a I/I form, which has a loop III, but lacks loop I, because the 5′ and 3′ termini are located at the end of stem I. Stem I of Schistozyme contains a bulge. Mutants were constructed of both the cis-cleaving form of Schistozyme and the trans-cleaving form of Schistozyme. Each mutant changes four nucleotides in the bulge within stem I (see FIG. 11).

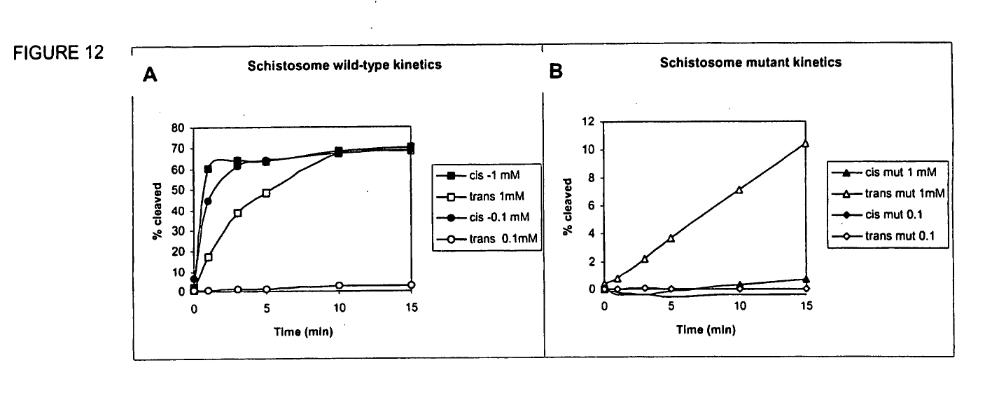

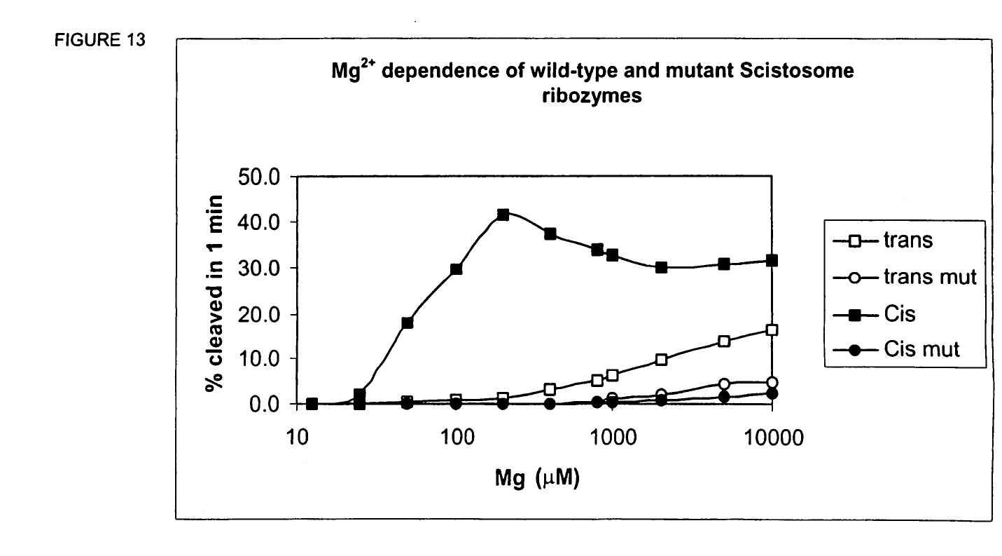

As shown in FIG. 12 and Table 3, removal of the bulge within stem I of the cis-cleaving Schistozyme results in a decrease in cleavage activity of 2 orders of magnitude at 1 mM Mg²⁺. At 0.1 mM Mg²⁺, cleavage is nearly abolished in both the cis-cleaving and trans-cleaving mutant Schistozymes. The effect of Mg²⁺ concentration on the activity of wild-type and mutant Schistozymes is shown in FIG. 13. One possible explanation for the decreased activity of the mutants is that mutation of the nucleotides in the bulge within stem I negatively affects an interaction between that bulge and loop II. Another possible explanation is that, because the nucleotides were mutated to base pair with the other strand of the symmetric bulge, the increased complementarity of stem I increased the melting temperature and slowed product release following cleavage. These possible explanations, however, are in no way limiting, and such interactions and/or melting temperature differences are not required.

TABLE 3
Schistozyme wild-type and mutant kinetics

| ribozyme | Mg²⁺ conc. | k_{obs} (min⁻¹) | fold loss of activity |
|---|---|---|---|
| cis | 1 mM | 3.2 | 1 |
| trans | 1 mM | 3.3 | 0.96 |
| cis mutant | 1 mM | 1.84 × 10⁻² | 173 |
| trans mutant | 1 mM | 2.359 × 10⁻¹ | 13 |
| cis | 0.1 mM | 3.3 | 0.97 |
| trans | 0.1 mM | 5.72 × 10⁻² | 55 |
| cis mutant | 0.1 mM | nd | >10000 |
| trans mutant | 0.1 mM | nd | >10000 |

### Example 6

### Trans-Cleaving Schistozyme Hammerhead Ribozyme Mutants

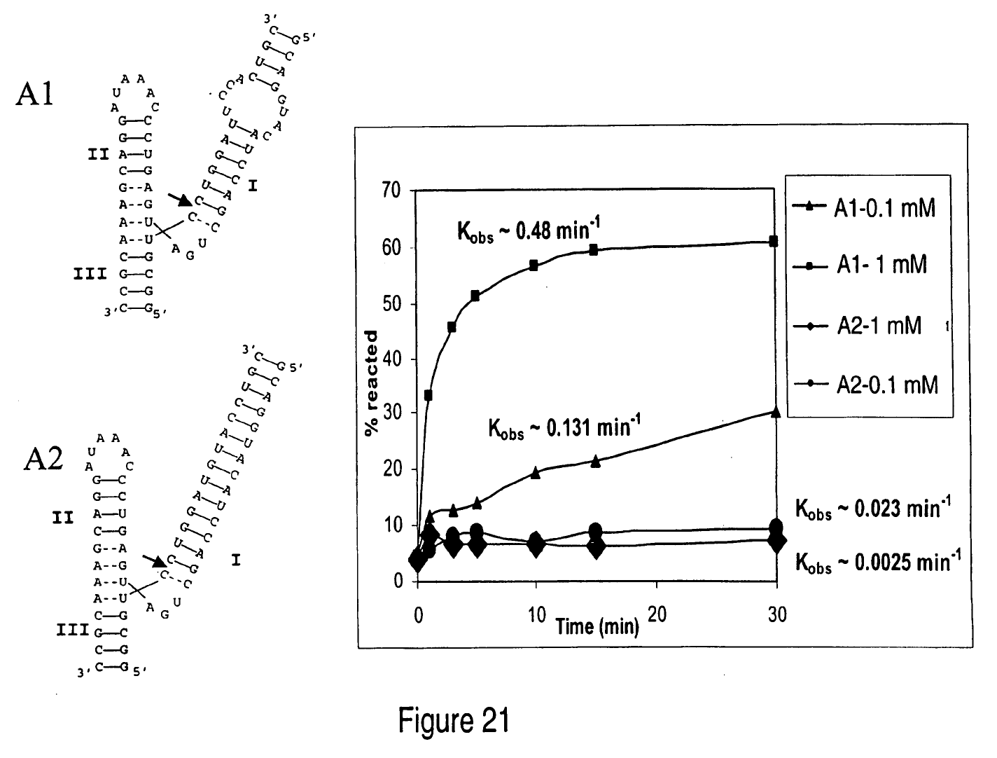

The Schistozyme cis-cleaving hammerhead ribozyme was converted to a trans-cleaving hammerhead ribozyme by removing loop III (FIG. 21, A1). A mutant Schistozyme trans-cleaving ribozyme, which lacks the bulge within stem I, was also made (FIG. 21, A2). In FIG. 21, A1 and A2, the substrate strand is the strand that has its 5′ terminus at the end of stem III and its 3′ terminus at the end of stem I.

Synthetic RNAs corresponding to the substrate strand and the ribozyme strand of each of the trans-cleaving hammerhead ribozymes were purchased from Dharmacon (Boulder, Colo.). To monitor the cleavage of the substrate strand, the substrate strand was radiolabeled with ³²P-y-ATP using T4 Polynucleotide Kinase (T4PK) by standard methods, and the labeled RNA was purified by denaturing PAGE.

Reactions were carried out in 0.1 mM Mg²′ and 50 mM Tris-HCl (pH 7.0) at 37° C. Reactions were stopped by addition of 2 volumes of loading buffer (7M urea, 95% formamide, 30 mM EDTA). The extent of cleavage was determined after separating the reaction products on a 10% polyacrylamide gels under denaturing conditions. The starting materials and the cleavage products were quantitated using a Molecular Dynamics Phosphoimager. The initial rate, K_{obs}, was calculated for each ribozyme by plotting the percent cleaved versus time using KaleidaGraph™ software (Synergy Software) and using the method described in McCaffrey et al. (1999) in Intracellular Ribozyme Applications: Principles and Protocols, Horizon Scientific Press, pp. 57-68, which is incorporated herein by reference for any purpose.

As shown in FIG. 21, eliminating the bulge within stem I of the Schistozyme trans-cleaving ribozyme resulted in almost a 200-fold loss of activity at 0.1 mM Mg²⁺ (˜0.48 min⁻¹ for the Schistozyme trans-cleaving ribozyme versus ˜0.0025 min⁻¹ for the mutant Schistozyme trans-cleaving ribozyme). At 1 mM Mg²⁺, the loss in activity was about 5-fold (˜0.131 min⁻¹ for the Schistozyme trans-cleaving ribozyme versus ˜0.023 min⁻¹ for the mutant). One possible explanation for these results is that the bulge within stem I contributes to effective catalysis at low Mg²⁺ concentrations. This possible explanation, however, is in no way limiting, and such a contribution is not required.

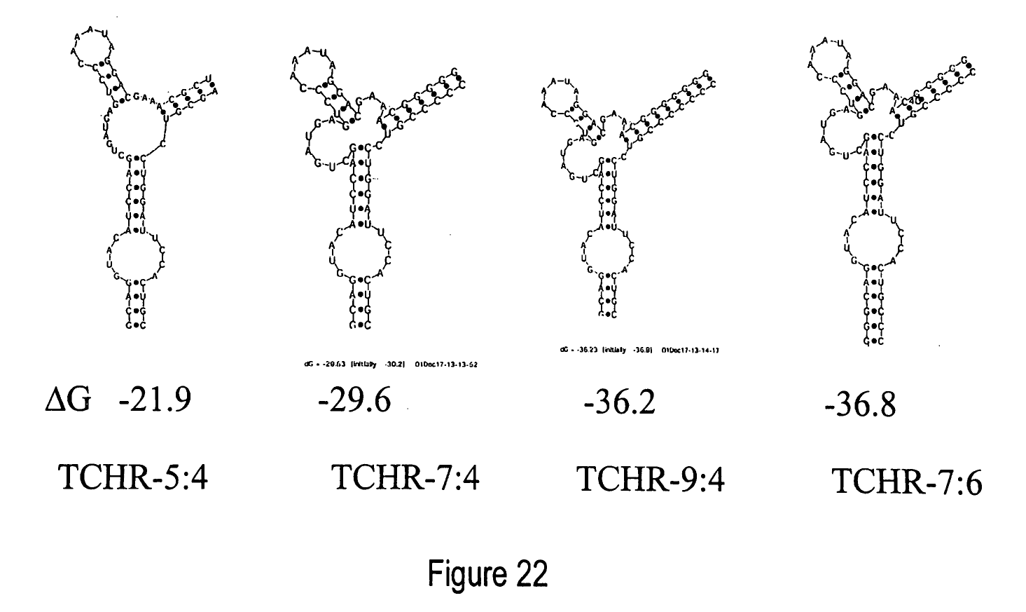

Next, the effect of altering the length of stems I and II was studied using the Schistozyme trans-cleaving ribozyme. As shown in FIG. 22, four variants were designed: TCHR-5:4 had 5 base pairs in stem III and 4 base pairs after the bulge within stem I; TCHR-7:4 had 7 base pairs in stem III and 4 base pairs after the bulge within stem I; TCHR-9:4 had 9 base pairs in stem III and 4 base pairs after the bulge within stem I; and TCHR-7:6 had 7 base pairs in stem III and 6 base pairs after the bulge within stem I.

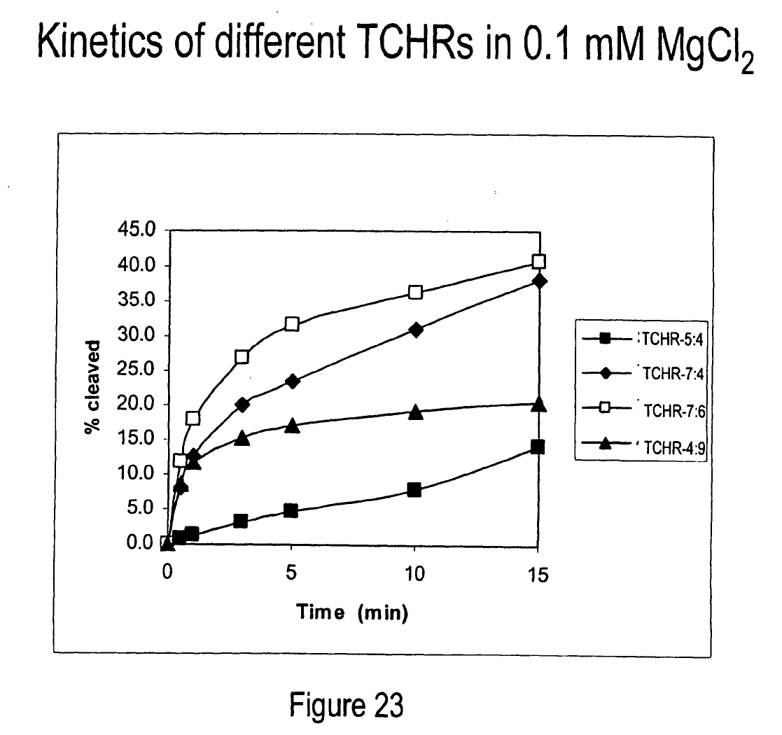

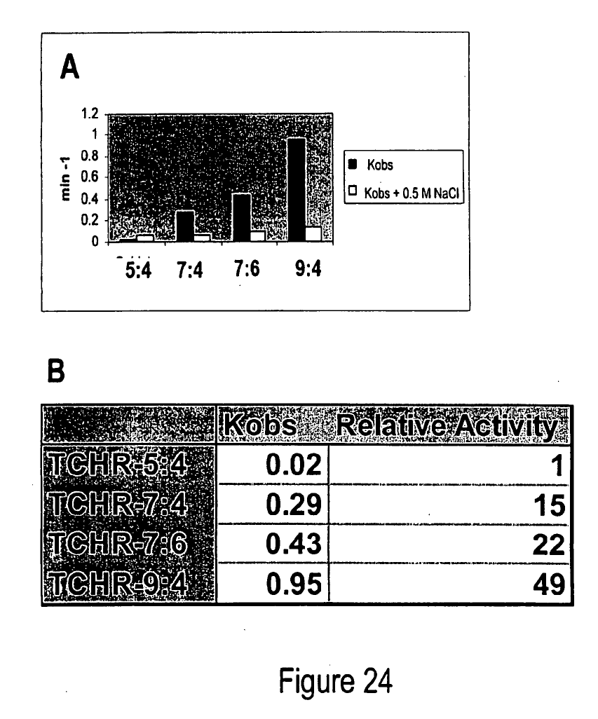

The activity of each of the four variants was measured as described above for the trans-cleaving Schistozyme and the mutant Schistozyme, in a buffer comprising 50 mM Tris-HCl (pH 7.0) and 0.1 mM MgCl₂. FIG. 23 shows that TCHR-7:6 reached the highest percent cleaved in fifteen minutes, followed by TCHR-7:4, then TCHR-9:4, and then TCHR-5.4. As shown in FIG. 24A, however, the initial rate was fastest for TCHR-9:4, followed by TCHR-7:6, then TCHR-7:4, and then TCHR-5:4. Therefore, it appears that in certain embodiments, ribozymes with longer stems have faster initial cleavage rates. These results, however, show that in certain embodiments, certain larger stems result in a reduction of the percent cleavage (see, e.g., TCHR-9:4). These results also showed that in certain embodiments, shorter stems may also result in lower percent cleavage.

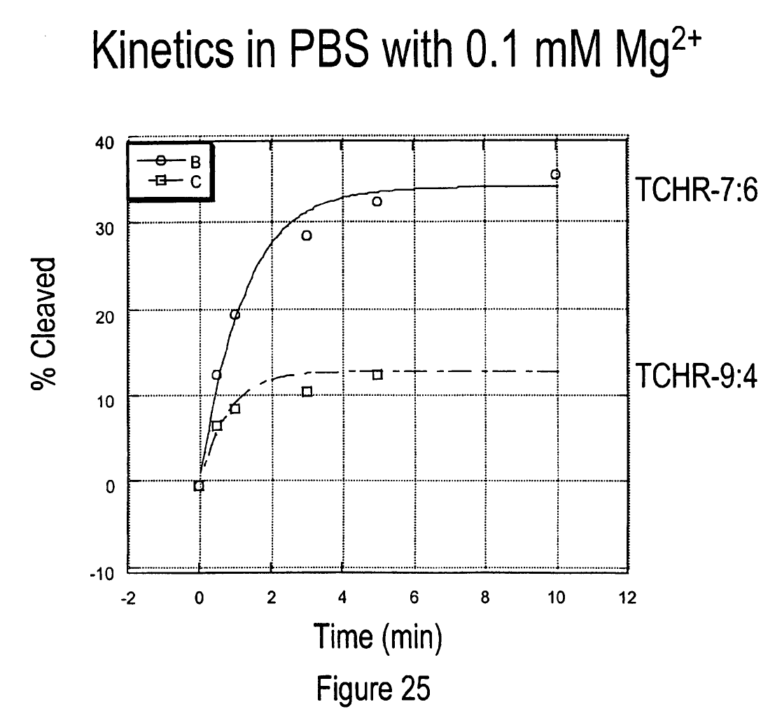

The previous studies were conducted in the absence of monovalent ions. To study the effect of such ions on ribozyme cleavage in certain embodiments, cleavage reactions with TCHR-5:4, TCHR-7:4, TCHR-9:4, and TCHR-7:6 were carried out in a buffer containing 50 mM Tris-HCl (pH 7.0), 0.1 mM Mg²⁺, and 0.5 M NaCl. As shown in FIG. 24A, the initial rate of cleavage in the presence of 0.5 M NaCl in this experiment was less dependent on the length of stems I and III and was generally slower than cleavage in the absence of NaCl. However, the percent cleavage was slightly greater than the percent cleavage without NaCl, and the percent cleavage differed with different lengths of stems I and III. A cleavage experiment with just two of the ribozyme variants performed in PBS containing 0.1 mM Mg²⁺, shown in FIG. 25, produced similar results. TCHR-9:4 resulted in a lower percent cleavage than TCHR-7:6.

### Example 7

### Comparison of TCHR Variants and Non-Natural Ribozyme HH16

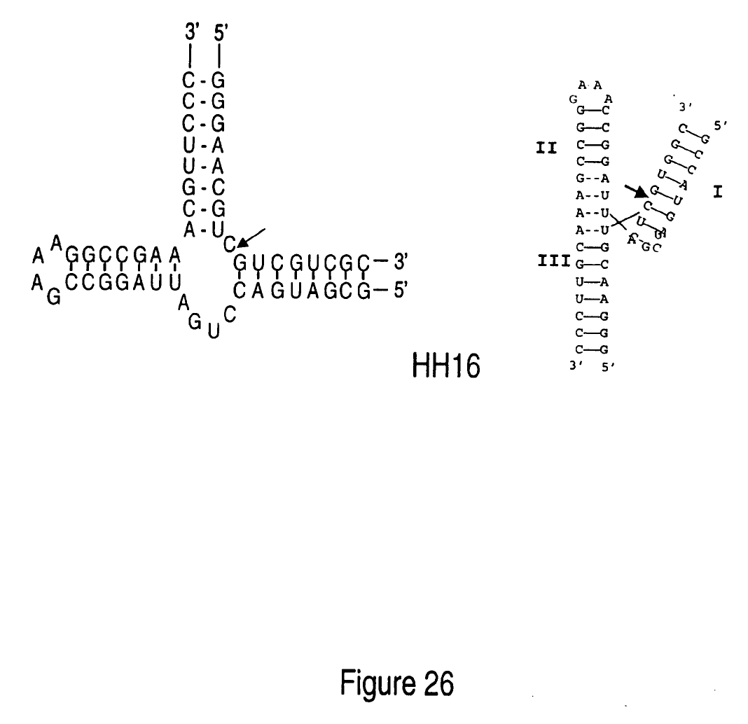

HH16 is a non-natural trans-cleaving hammerhead ribozyme disclosed, e.g., in Long et al. (1994) Proc. Natl. Acad. Sci. USA 91: 6977-6981. HH16 does not include a bulge within stem I. See FIG. 26.

A. Activity of HH16 and TCHR-5:4 in low Mg²⁺

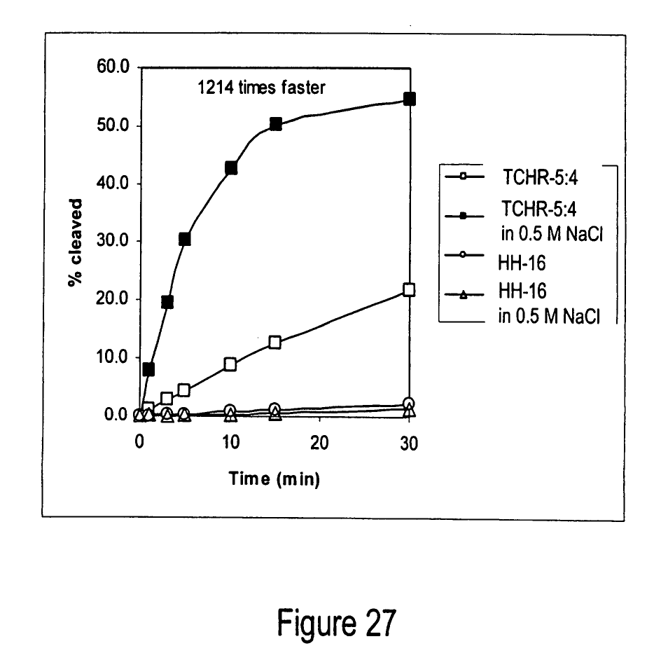

FIG. 27 shows a comparison of the activity of HH16 to the activity of TCHR-5:4 in 50 mM Tris-HCl (pH 7.0) and 0.1 mM Mg²⁺, with and without 0.5 M NaCl. HH16 showed nearly undetectable cleavage during a 30 minute incubation at 37° C. in 0.1 mM MgCl₂, regardless of the presence of 0.5 M NaCl. In contrast, TCHR-5:4 reached greater than 50% cleavage within 15 minutes in the presence of 0.5 M NaCl, with a K_{obs} of 0.5 min⁻¹. In the absence of NaCl, TCHR-5:4 cleavage is slower.

B. Activity of HH16 and TCHR-5:4 at pH 5.5 and DH 6.0

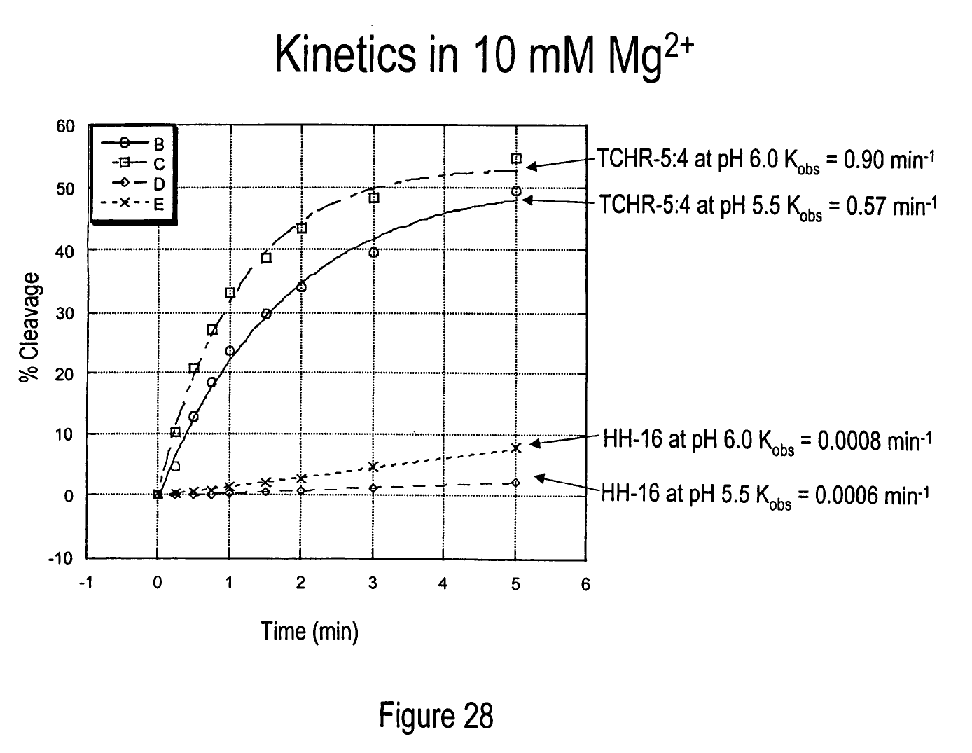

To analyze certain kinetics of TCHR-5:4 and HH16, their activity at pH 6.0 and pH 5.5 were measured in 10 mM MgCl₂. As shown in FIG. 28, cleavage of TCHR-5:4 is faster than cleavage of HH16 under both pH conditions.

As shown in FIG. 21, the elimination of the bulge within stem I of the Schistozyme trans-cleaving hammerhead ribozyme resulted in a decrease in the activity. One possible explanation for the lower activity of HH16 is its lack of a bulge within stem I. As discussed previously, a possible explanation for better activity when a bulge is present within stem I is a possible interaction between loop II and a bulge within stem I. This possible explanation, however, is in no way limiting, and such interactions are not required.

C. Activity of HH16 and TCHR-5:4 in LiCl

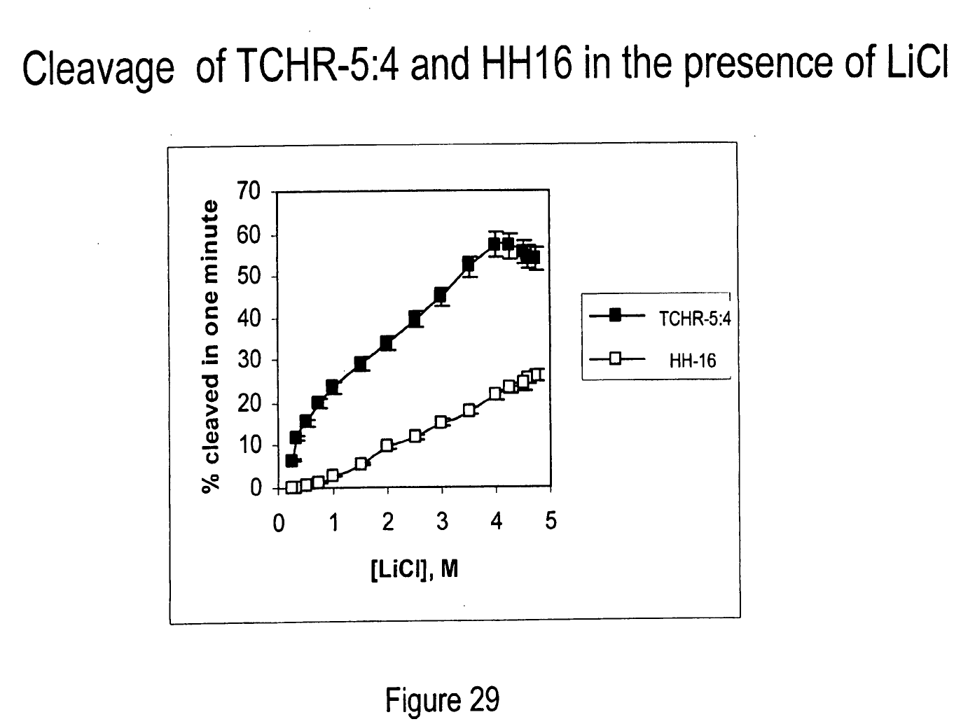

High concentrations of monovalent ions have also been shown to induce the cleavage reaction in ribozymes (see, e.g., Curtis et al. (2001) RNA 7: 546-552; O'Rear et al. (2001) RNA 7: 537-545). To determine whether HH16 and TCHR-5:4 differ in their ability to cleave in the presence of only monovalent ions, cleavage reactions were performed in buffer containing various concentrations of LiCl and 10 mM EDTA to substantially eliminate free Mg⁺² ions. As shown in FIG. 29, HH16 showed a linear relationship between the percent cleaved in one minute and the concentration of LiCl. This result is consistent with the previous reports (see, e.g., Curtis et al. (2001) RNA 7: 546-552; O'Rear et al. (2001) RNA 7: 537-545).

In contrast, TCHR-5:4 showed a greater percent cleavage over all concentrations LiCl, as compared to HH16. Furthermore, the activity of TCHR-5:4 reached a plateau at concentrations of 4 M LiCl or greater, while HH16 did not plateau over all concentrations used. One possible explanation for the better activity of TCHR-5:4 in LiCl as compared to HH16 is that the bulge within stem I of TCHR-5:4 may interact with loop II and contribute to greater activity, and this interaction may not require a high concentration of counter ions. This possible explanation, however, is in no way limiting, and such interactions are not required.

D. Effect of DH on the activity of HH16 and TCHR-5:4 in low Mg²⁺

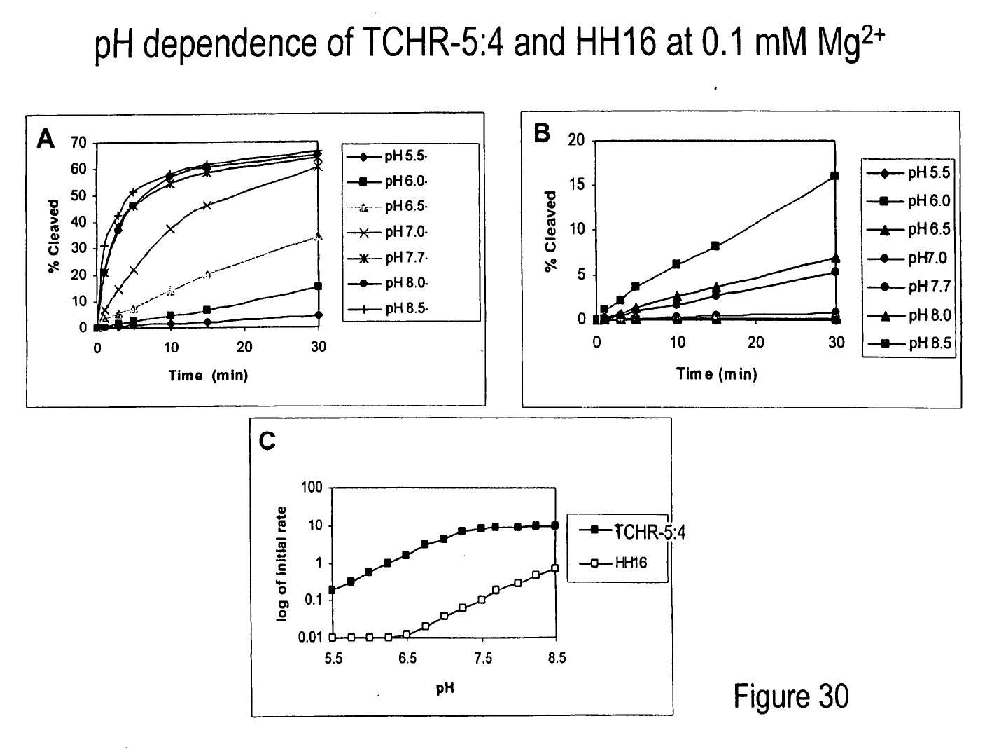

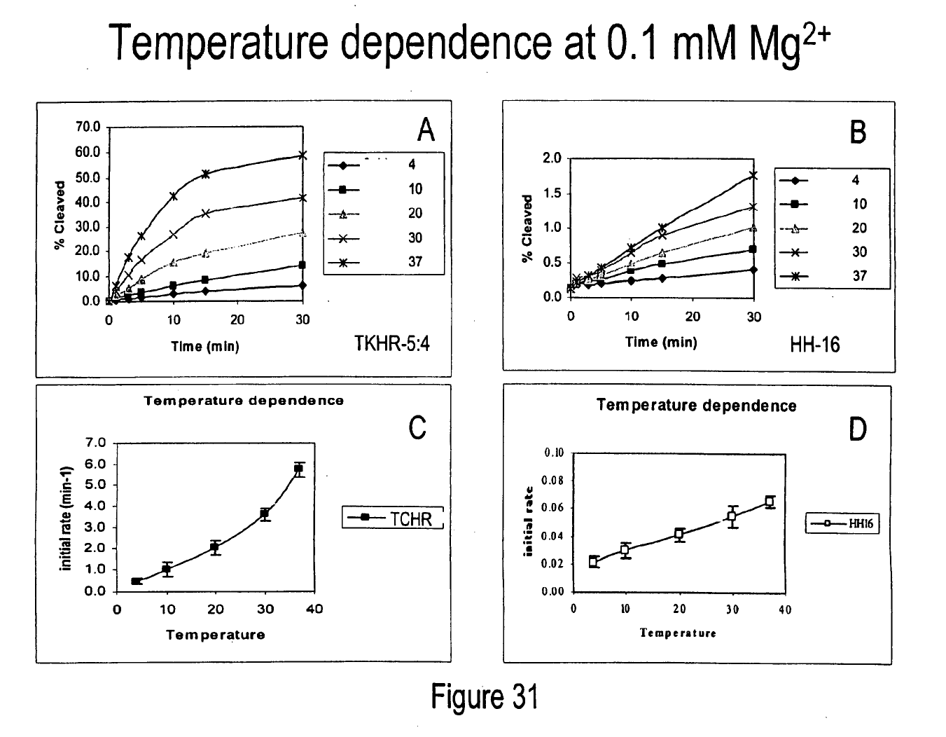

To investigate the effect of pH on the activity of HH16 and TCHR-5:4 in certain embodiments, cleavage reactions were carried out in the presence of 0.1 mM MgCl₂ in a series of buffers with various pH values. Reactions with a pH of 5.5, 6.0, or 6.5 were carried out in 50 mM MES (2-[N-Morpholino]ethanesulfonic acid), which has a pKa of 6.1. Reactions with a pH of 7.0 or 7.7 were carried out in 50 mM MOPS (3-[M-Morpholino]propanesulfonic acid), which has a pKa of 7.2. Reactions with a pH of 8.0 or 8.5 were carried out in 50 mM TAPS (N-tris[hydroxymethyl]methyl-3-aminopropanesulfonic acid), which has a pKa of 8.4. As shown in FIG. 30A, TCHR-5:4 had similar activity in buffers at pH 7.7, pH 8.0, and pH 8.5. The activity of TCHR-5:4 was slightly decreased at pH 7.0, and decreased further at pH values that were tested that were below pH 7.0. In contrast, as shown in FIG. 30B, the activity of HH16 was greatest at pH 8.5 and decreased from pH 8.5 to pH 8.0, from pH 8.0 to pH 7.7, and from pH 7.7 to pH 7.0. HH16 had nearly undetectable activity at pH 7.0 or lower.

FIG. 30C shows a plot of the logarithm of the initial rate of cleavage of TCHR-5:4 and HH16 versus the pH. TCHR-5:4 reached a plateau at about pH 7.0, while HH16 did not plateau over the pH range tested.

E. Effect of Temperature on the Activity of HH16 and TCHR-5:4 in Low Mg²⁺

The initial cleavage rate of HH16 and TCHR-5:4 was studied as a function of temperature in a buffer containing 50 mM Tris-HCl (pH 7.0) and 0.1 mM MgCl₂. As shown in FIGS. 31A and 31B, the percent cleavage of TCHR-5:4 increased exponentially at 30° C. and 37° C., whereas the percent cleavage of HH16 increased linearly at those temperatures. Furthermore, the percent cleavage of HH16 after 30 minutes was less than one-tenth the percent cleavage of TCHR-5:4 at all temperatures tested. Both ribozymes showed decreased activity as the tested temperature values decreased.

As shown in FIGS. 31C and 31D, the initial rate of TCHR-4:5 cleavage was at least 10 fold higher than the initial rate of HH16 cleavage at each temperature tested. The initial rate of cleavage for both ribozymes increased with increased temperature over all temperatures tested. One possible explanation for the greater activity of TCHR-5:4 at all temperatures tested is that TCHR-5:4 has a lower activation energy for cleavage than HH16, and that this lower activation energy is due to an interaction between loop II and the bulge within stem I in TCHR-5:4. This possible explanation, however, is in no way limiting, and such interactions are not required.

F. Effect of Mg²⁺ Concentration on the Activity of HH16 and TCHR-5:4

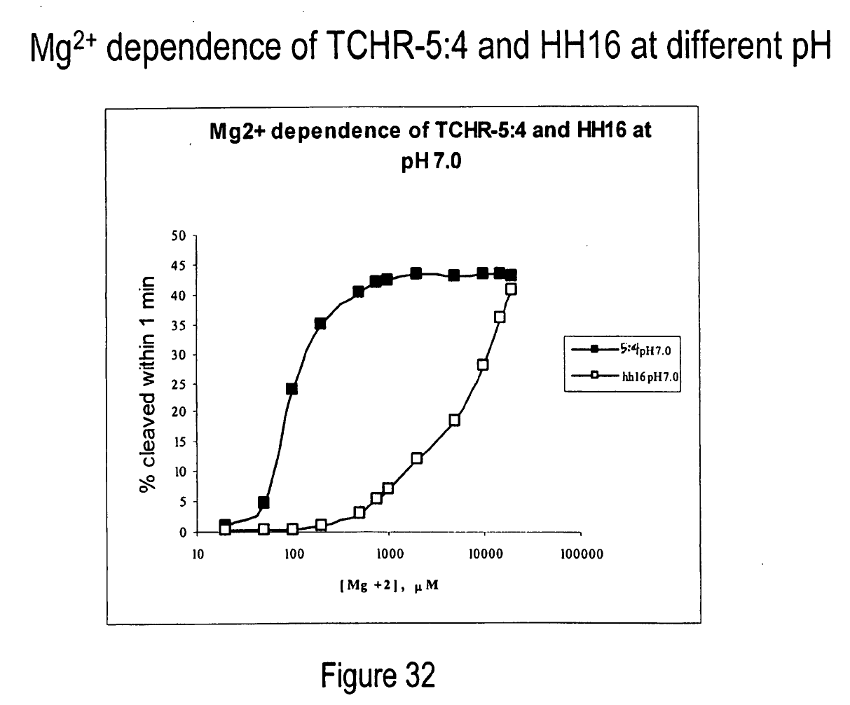

The effect of Mg²⁺ concentration on the activity of HH16 and TCHR-5:4 was studied over a MgCl₂ concentration range of 0.02 mM to 20 mM. Each reaction was carried out in a buffer containing 50 mM Tris-HCl (pH 7.0) at 37° C. for one minute. As shown in FIG. 32, there was a difference in the effect of Mg²⁺ concentration between HH16 and TCHR-5:4. TCHR-5:4 reached a maximum percent cleavage at about 1 mM Mg²⁺, while HH16 did not reach a maximum over the range studied. The activity of HH16 was lower than the activity of TCHR-5:4 at most of the Mg²⁺ concentrations tested, and only approached the activity of TCHR-5:4 at 10 mM Mg²⁺. As discussed previously, one possible explanation for this observation is that loop II and the bulge within stem I of TCHR-5:4 interact and stabilize an active conformation, resulting in higher activity at lower Mg²⁺ concentrations. This possible explanation, however, is in no way limiting, and such interactions are not

G. Activity of TCHR-5:4 with an Asymmetric Stem I Bulge

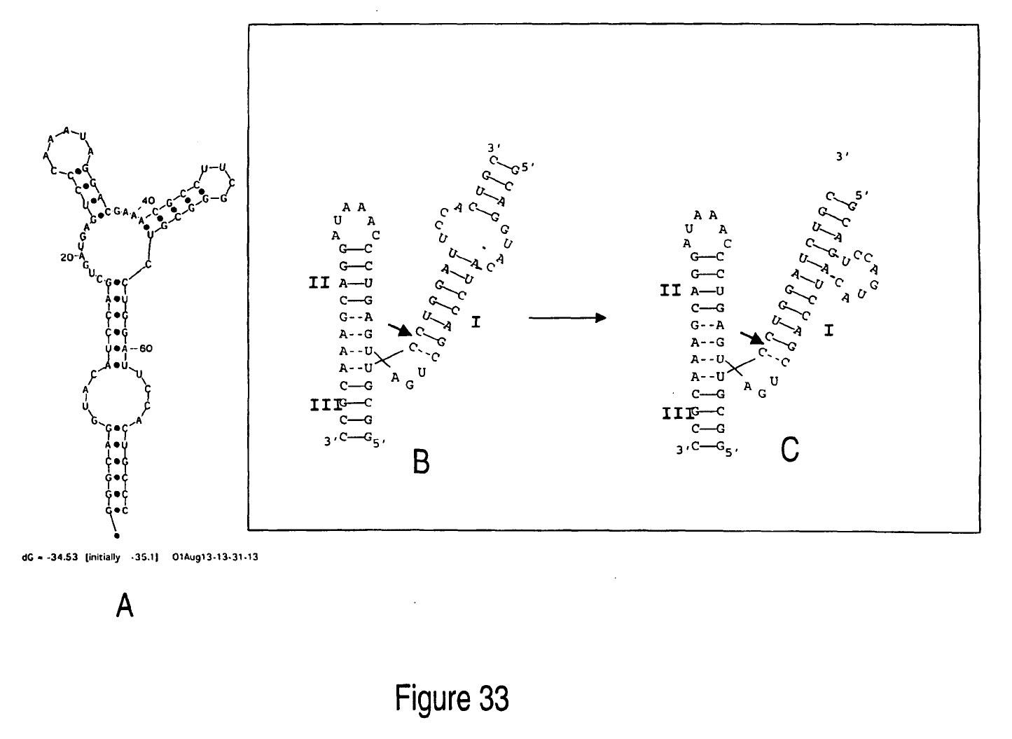

A TCHR-5:4 variant was designed that converted the symmetric bulge of TCHR-5:4 to an asymmetric bulge (see FIG. 33). The asymmetric bulge contains the eight nucleotides of the symmetric bulge.

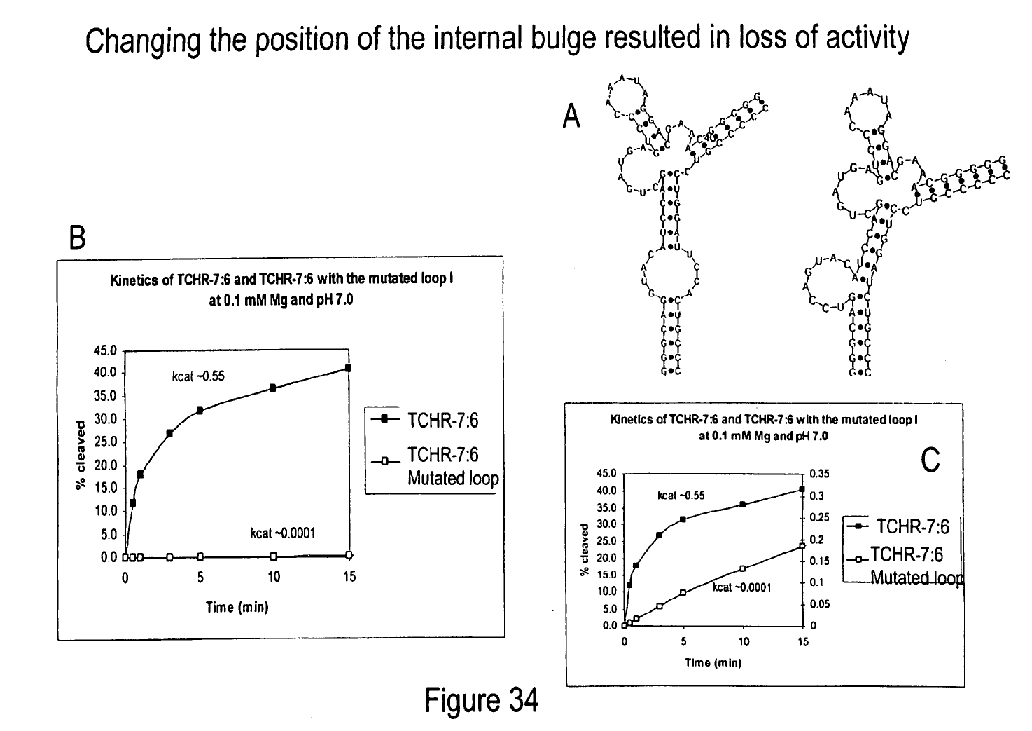

As shown in FIG. 34B, converting the symmetric bulge of TCHR-5:4 to an asymmetric bulge nearly abolished cleavage activity (FIG. 34C shows the same data as FIG. 34B, except the data for TCHR-5:4 with an asymmetric bulge is plotted against a separate scale, shown on the right side of the graph).

### Example 8

### In Vitro Selection of Hammerhead Ribozymes

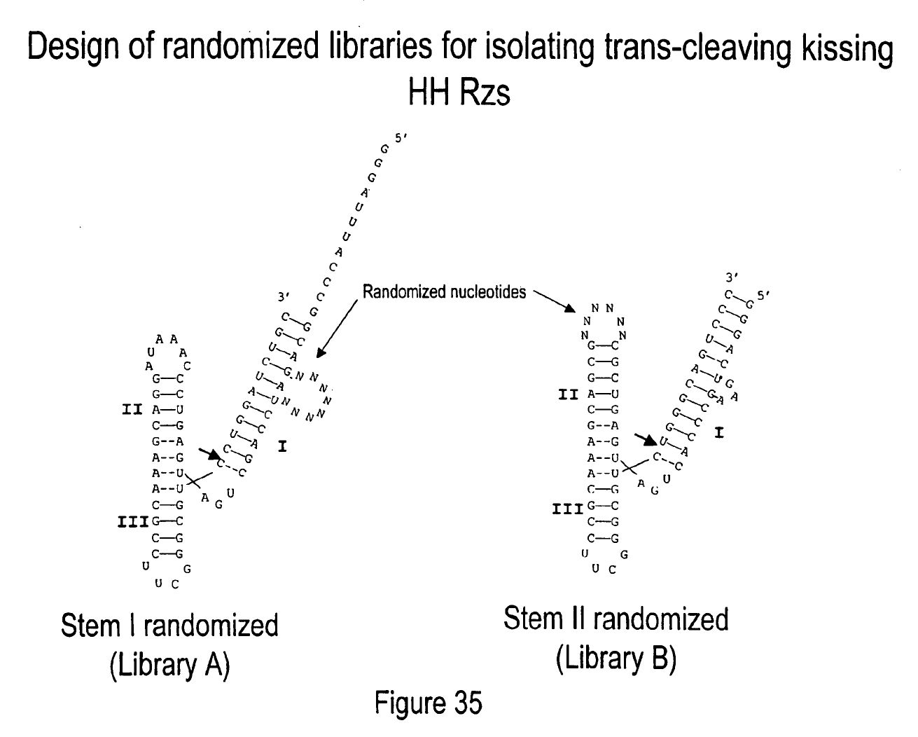

An asymmetric bulge may be useful in the design of trans-cleaving hammerhead ribozymes, in which one strand of the ribozyme has the sequence of the desired target RNA. To select a I/III trans-cleaving hammerhead ribozyme having an asymmetric bulge within stem I that has activity, a library of I/I cis-cleaving hammerhead ribozymes was designed that had an asymmetric bulge within stem I, as shown in FIG. 35. Once a cis-cleaving hammerhead ribozyme is selected, it may be possible to remove loop III to create a trans-cleaving hammerhead ribozyme.

In Library A, the asymmetric bulge within stem I had eight nucleotides that were randomized and the remainder of the ribozyme was the same across the members of the library. In Library B, the six nucleotides in loop II were randomized and the remainder of the ribozyme was the same across the members of the library. In both libraries, the asymmetric bulge within stem I was located six base pairs from the core. The complexity of each library was about 10⁵ individual sequences.

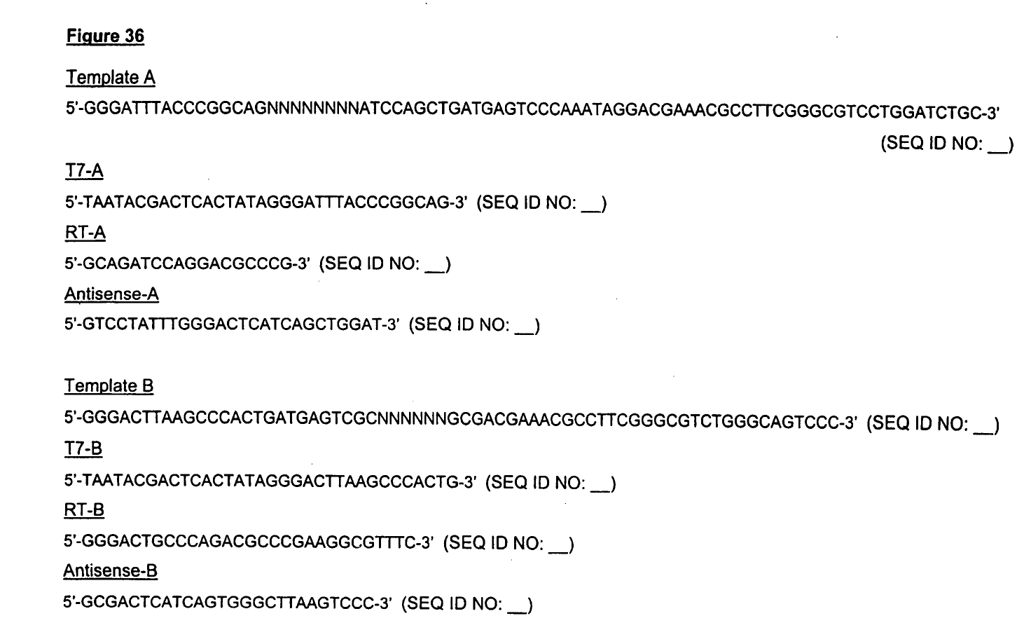

Each ribozyme library was transcribed from its corresponding DNA template in the presence of an antisense oligonucleotide that hybridized to the core. The antisense oligonucleotide was used to prevent or reduce cleavage during the transcription reaction (see FIG. 36 for the template and antisense oligonucleotide sequences). Full-length ribozyme molecules were separated from incomplete transcription products by denaturing polyacrylamide gel electrophoresis (PAGE) and purified from the gel as follows. Gel sections containing full-length ribozymes were cut out, crushed, and eluted into 300 mM NaOAc (pH 5.5). The NaOAc solution was then filtered through a 0.2 micron filter to remove the crushed gel pieces, and the ribozymes were precipitated from the solution by addition of 3 volumes of 100% ethanol. The precipitated ribozymes were pelleted by centrifugation and pellets were washed 3 times with 75% ethanol and 2 times with 100% ethanol. The pellets were then dried and resuspended in 0.0002 mM EDTA. An equal volume of 2× selection buffer (100 mM Tris-HCl (pH 7.0) and 0.2 mM MgCl₂; the final concentration in the selection reactions was therefore 50 mM Tris-HCl (pH 7.0) and 0.1 mM MgCl₂) was then added to the purified ribozymes and they were incubated at 37° C. for 10 minutes for the first selection cycle. For the second, third, fourth, fifth, sixth, and seventh selection cycles, the ribozymes were incubated for 10 min, 10 min, 5 min, 1 min, 0.5 min, and 0.5 min, respectively.

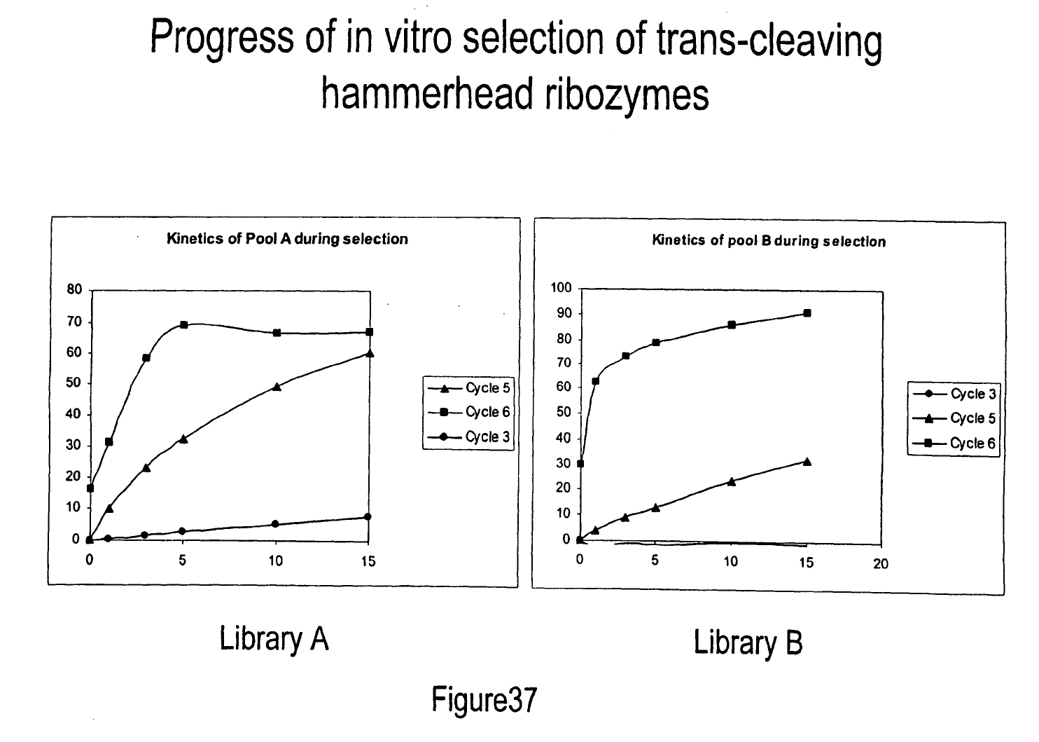

Following incubation, the cleaved ribozymes were separated from the uncleaved ribozymes by denaturing PAGE, purified from the gel, and then reverse transcribed into cDNA using RT-MLV (Epicenter Technologies), according the manufacturer's instructions. The cDNA was amplified by PCR for 10 to 20 cycles of 94° C. for 30 seconds, 55° C. for 45 seconds, and 72° C. for 60 seconds, using 2× Master Mix (Promega), according to the manufacturer's instructions. Following amplification, the PCR products were transcribed in the presence of antisense oligos and purified as described above. Seven rounds of selection were carried out. FIG. 37 shows the activity of the ribozyme pools obtained after the 3^{rd}, 5^{th}, and 6^{th} rounds of selection for Library A and for Library B.

To enrich for ribozymes with greater activity, the time of incubation was decreased in each successive round of selection as described above. After six rounds of selection, the percent cleavage of the pools of ribozymes from both libraries reached near saturation within about 5 minutes in 50 mM Tris-HCl (pH 7.0) and 0.1 mM MgCl₂, as shown in FIG. 37.

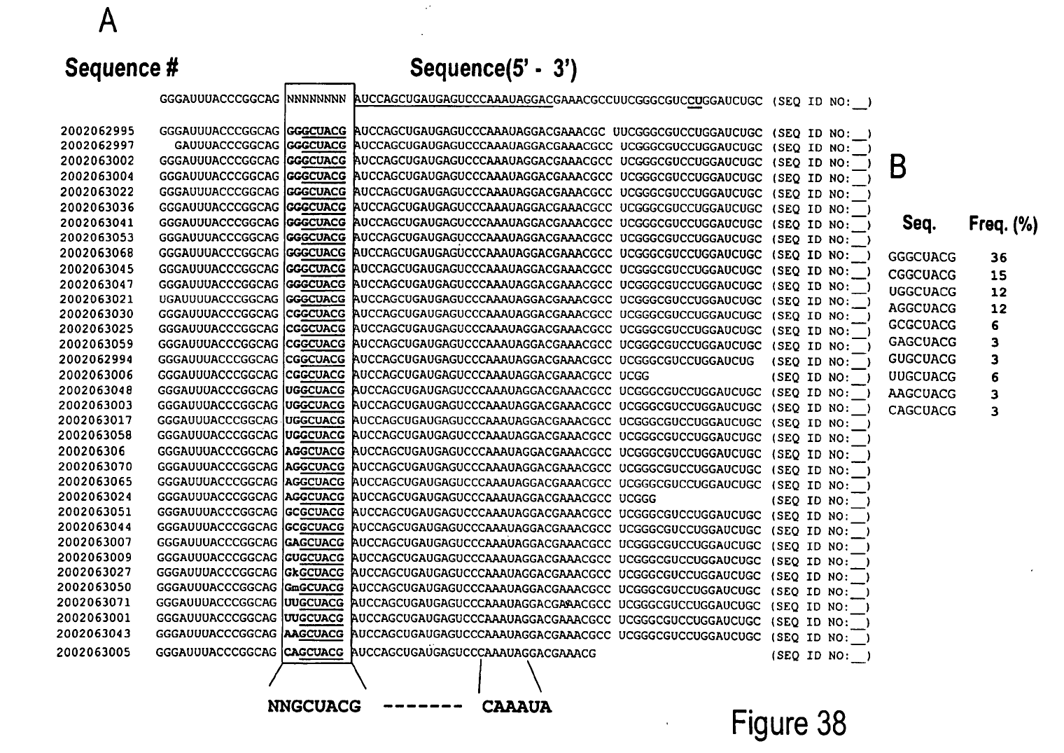

Following seven rounds of selection, the selected ribozymes were reverse transcribed and amplified by PCR, as described above. The PCR products were then cloned and sequenced by standard methods known in the art. Following seven rounds of selection, a consensus motif, NG/NGCUACG, was observed for the randomized bulge within stem I of library A (see FIG. 38); N in the first position means that that position is selected from A, C, G, and U; and G/N in the second position means that that position is selected from A, C, G, and U, although there is a preference for G. While the first two nucleotides showed some variability across the sequenced ribozymes, the last six nucleotides of all of the sequenced ribozymes was GCUACG. One possible explanation for the observed consensus motif is that it is capable of interacting with the fixed loop II sequence of the ribozyme library. This possible explanation, however, is in no way limiting, and such interactions are not required.

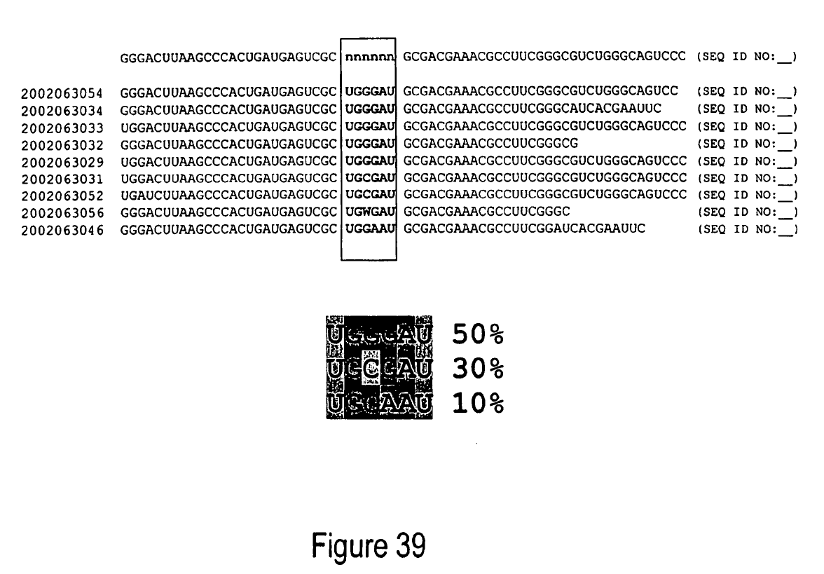

Following seven rounds of selection, a consensus motif, UGG/CG/AAU, was observed for the randomized loop II of library B (see FIG. 39). G/C in the third position means that that position is selected from G and C, and G/A in the fourth position means that that position is selected from G and A. One possible explanation for the observed consensus motif is that it is capable of interacting with the fixed bulge sequence within stem I of the ribozyme library. This possible explanation, however, is in no way limiting, and such interactions are not required.

### Example 9

### Activity of Certain Individual Selected Hammerhead Ribozymes

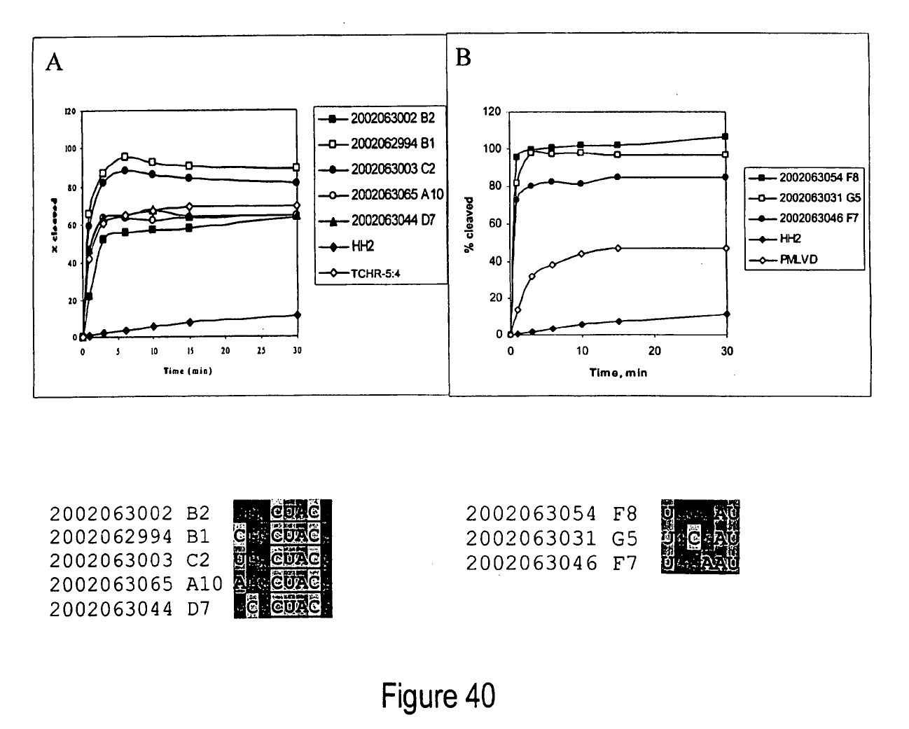

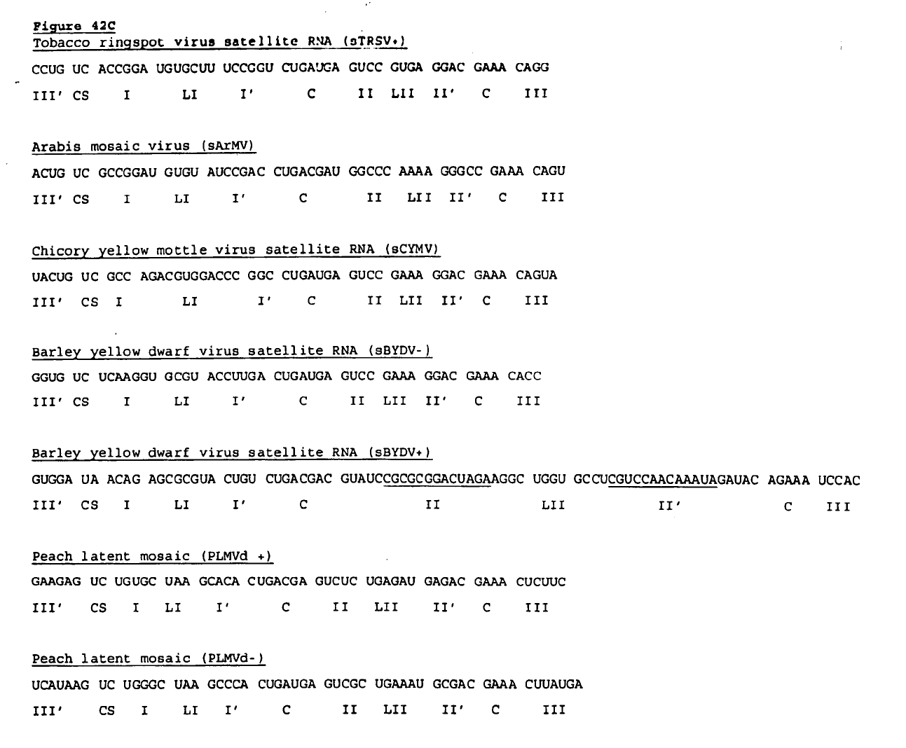

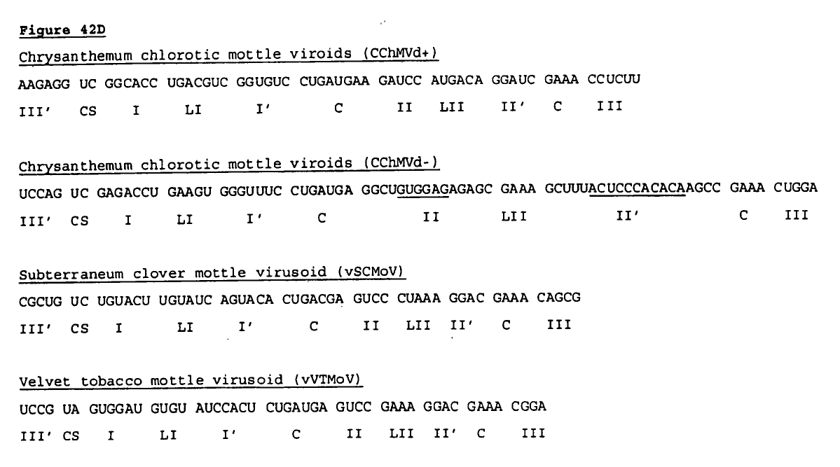

Several individual in vitro selected hammerhead ribozymes were transcribed and the activity of each was determined in 50 mM Tris-HCl (pH 7.0) and 0.1 mM Mg²⁺. FIG. 40A shows the activity of five of the ribozymes selected from library A, along with the sequence of the bulge within stem I for each. FIG. 40B shows the activity of three of the ribozymes selected from library B, along with the loop II sequence for each. HH2, which is a cis-cleaving version of HH16 (see FIG. 42), was included in each experiment for comparison. In addition, TCHR-5:4 (FIG. 40A) and PLMVd (FIG. 40B) were also included for comparison.

Each of the selected ribozymes, as well as TCHR-5:4 and PLMVd, reached a maximum percent cleavage in less than 5 minutes. The ribozymes selected from library A reached 55% to about 90% cleavage in about 5 minutes, while TCHR-5:4 reached a maximum percent cleavage of about 65% in about 5 minutes. HH2, on the other hand, reached a maximum percent cleavage of only about 10% in 30 minutes. The ribozymes selected from library B had greater activity, and reached between about 80% and 100% cleavage after only 5 minutes, while PLMVd reached about 40% cleavage in 5 minutes. HH2 again reached only about 10% cleavage in 30 minutes. These data demonstrate that the selection method succeeded in producing active hammerhead ribozymes.

We claim:

1. A non-natural cis-cleaving hammerhead ribozyme comprising a core, a stem I, a stem II, a stem III, a loop I, and a loop II, wherein loop I and loop II are derived from loop I and loop II of a first hammerhead ribozyme selected from cherry small circular RNA+ (Scc+), cherry small circular RNA− (Scc−), Lucerne transient streak virusoid+ (sLTSV+), Lucerne transient streak virusoid− (sLTSV−), Tobacco ringspot virus satellite RNA+ (sTRSV+), Arabis mosaic virus (sArMV), Chicory yellow mottle virus satellite RNA (sCYMV), Barley yellow dwarf virus satellite RNA− (sBYDV−), Barley yellow dwarf virus satellite RNA+ (sBYDV+), Peach latent mosaic virus RNA+ (PLMVd+), Peach latent mosaic virus RNA− (PLMVd−), Chrysanthemum chlorotic mottle viroid+ (CChMVd+), Chrysanthemum chlorotic mottle viroid− (CChMVd−), Subterraneum clover mottle virusoid (vSCMoV), and velvet tobacco mottle virusoid (vVTMoV), and wherein at least one of stem I, stem II, and stem III is derived from a second hammerhead ribozyme that is not the same as the first hammerhead ribozyme.

2. A non-natural cis-cleaving hammerhead ribozyme comprising a core, a stem I, a stem II, a stem III, a bulge within stem I, and a loop II, wherein loop II and the bulge within stem I are derived from loop II and a bulge within stem I of a first hammerhead ribozyme selected from Notophthalmus viridescens satellite RNA (newt), Ambystoma talpoideum (Am. ta.), Amphiuma tridactylum (Am. tr.), Schistosoma mansoni hammerhead ribozyme (Schistozyme), D. baccettii cricket hammerhead ribozyme (cricketzyme A), D. schiavazzii cricket hammerhead ribozyme (cricketzyme B), and Avocado sunblotch viroid+ (ASBV+), and wherein at least one of stem II, stem III, and a portion of stem I is derived from a second hammerhead ribozyme that is not the same as the first hammerhead ribozyme.

3. A non-natural cis-cleaving hammerhead ribozyme comprising a core, a stem I, a stem II, a stem III, loop I, and a bulge within stem II, wherein loop I and the bulge within stem II are derived from a first ribozyme selected from Chrysanthemum chlorotic mottle viroid− (CChMVd−) and Barley yellow dwarf virus satellite RNA (sBYDV+), and wherein at least one of stem I, stem III, and a portion of stem II is derived from a second hammerhead ribozyme that is not the same as the first hammerhead ribozyme.

4. A non-natural cis-cleaving hammerhead ribozyme comprising a core, a stem I, a stem II, a stem III, a bulge within stem I, and a bulge within stem II, wherein the bulge within stem I and the bulge within stem II are derived from a first ribozyme selected from Avocado sunblotch viroid− (ASBV−) and Carnation small viroid-like RNA+ (CarSV+), and wherein at least one of stem III, a portion of stem I, and a portion of stem II is derived from a second hammerhead ribozyme that is not the same as the first hammerhead ribozyme.

5. A non-natural cis-cleaving hammerhead-ribozyme comprising the structure III: 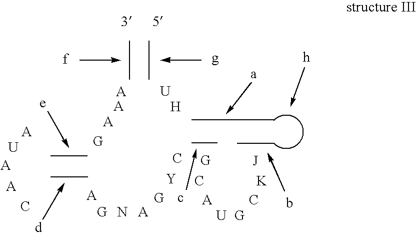
wherein:
J, K, and N are each independently selected from A, C, G, and U;
Y is selected from C and U;
H is selected from A, C, and U;
a is a sequence of 4-20 nucleotides, wherein each nucleotide is independently selected from A, C, G, and U;
b, c, d, e, f, and g are each a sequence of 2-20 nucleotides, wherein each nucleotide is independently selected from A, C, G, and U;
h is a sequence of 1-20 nucleotides, wherein each nucleotide is independently selected from A, C, G, and U;
a, b, JKCGUACG, and c together are stem I;
d and e together are stem II; and
f and g together are at least a portion of stem III.

6. A non-natural cis-cleaving hammerhead ribozyme comprising the structure IV: 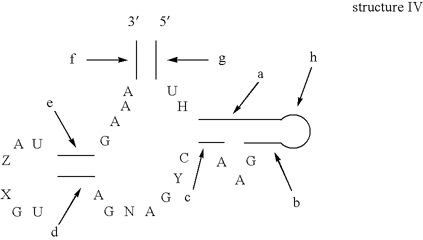
wherein:
N is selected from A, C, G, and U;
Y is selected from C and U;
X is selected from G and C;
Z is selected from G and A;
H is selected from A, C, and U;
a is a sequence of 4-20 nucleotides, wherein each nucleotide is independently selected from A, C, G, and U;
b, c, d, e, f, and g are each a sequence of 2-20 nucleotides, wherein each nucleotide is independently selected from A, C, G, and U;
h is a sequence of 1-20 nucleotides, wherein each nucleotide is independently selected from A, C, G, and U;
a, b, GAA, and c together are stem I;
d and e together are stem II; and
f and g together are stem III.

7. A non-natural cis-cleaving hammerhead ribozyme comprising: the structure VI: 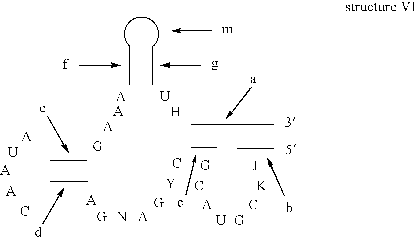
wherein:
J, K, and N are each independently selected from A, C, G, and U;
Y is selected from C and U;
H is selected from A, C, and U;
a is a sequence of 4-20 nucleotides, wherein each nucleotide is independently selected from A, C, G, and U;
b, c, d, e, f, and g are each a sequence of 2-20 nucleotides, wherein each nucleotide is independently selected from A, C, G, and U;
m is a sequence of 1-20 nucleotides, wherein each nucleotide is independently selected from A, C, G, and U;
a, b, JKCGUACG, and c together are stem I;
d and e together are stem II; and
f and g together are at least a portion of stem III.

8. A non-natural cis-cleaving hammerhead ribozyme comprising the structure VII: 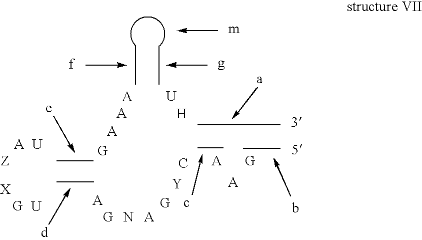
wherein:
N is selected from A, C, G, and U;
Y is selected from C and U;
X is selected from G and C;
Z is selected from G and A;
H is selected from A, C, and U;
a is a sequence of 4-20 nucleotides, wherein each nucleotide is independently selected from A, C, G, and U;
b, c, d, e, f, and g are each a sequence of 2-20 nucleotides, wherein each nucleotide is independently selected from A, C, G, and U;
m is a sequence of 1-20 nucleotides, wherein each nucleotide is independently selected from A, C, G, and U;
a, b, GM, and c together are stem I;
d and e together are stem II; and
f and g together are stem III.

9. The non-natural cis-cleaving hammerhead ribozyme of any of claims 1-8, wherein the ribozyme cis-cleaves at an initial rate of at least 0.5 min⁻¹ in a buffer comprising 50 mM Tris (pH 7.0) and 1 mM Mg²⁺ at 37° C.

10. The non-natural cis-cleaving hammerhead ribozyme of any of claims 1-8, wherein the ribozyme cis-cleaves at an initial rate of at least 0.5 min⁻¹ in a buffer comprising 50 mM Tris (pH 7.0) and 0.5 mM Mg²⁺ at 37° C.

11. The non-natural cis-cleaving hammerhead ribozyme of any of claims 1-8, wherein the ribozyme cis-cleaves at an initial rate of at least 0.5 min⁻¹ in a buffer comprising 50 mM Tris (pH 7.0) and 0.1 mM Mg²⁺ at 37° C.

12. A polynucleotide comprising a first nucleic acid sequence, wherein the first nucleic acid sequence encodes the non-natural cis-cleaving hammerhead ribozyme of any of claims 1-8.

13. The polynucleotide of claim 12, further comprising a second nucleic acid sequence, wherein the second nucleic acid sequence encodes an RNA that is not a non-natural hammerhead ribozyme.

14. The polynucleotide of claim 13, wherein the first nucleic acid sequence is inserted in frame into the second nucleic acid sequence.

15. The polynucleotide of claim 13, wherein the second nucleic acid sequence comprises a non-coding region, wherein the non-coding region is selected from a 3′-untranslated region (3′-UTR), a 5′-untranslated region (5′-UTR), and an intron, and wherein the first nucleic acid sequence is inserted into the non-coding region.

16. A vector comprising the polynucleotide of claim 14.

17. A host cell comprising the polynucleotide of claim 16.

18. A vector comprising the polynucleotide of claim 15.

19. A host cell comprising with the polynucleotide of claim 18.

20. The non-natural hammerhead ribozyme of any of claims 5-8, wherein at least a portion of f and g together comprise an aptamer capable of binding a small molecule.

21. A polynucleotide comprising a first nucleic acid sequence, wherein the first nucleic acid sequence encodes the non-natural cis-cleaving hammerhead ribozyme of claim 20.

22. The polynucleotide of claim 21, further comprising a second nucleic acid sequence, wherein the second nucleic acid sequence encodes an RNA that is not a non-natural hammerhead ribozyme.

23. The polynucleotide of claim 22, wherein the first nucleic acid sequence is inserted in frame into the second nucleic acid sequence.

24. The polynucleotide of claim 22, wherein the second nucleic acid sequence comprises a non-coding region, wherein the non-coding region is selected from a 3′-untranslated region (3′-UTR), a 5′-untranslated region (5′-UTR), and an intron, and wherein the first nucleic acid sequence is inserted into the non-coding region.

25. A vector comprising the polynucleotide of claim 23.

26. A host cell comprising with the polynucleotide of claim 25.

27. A vector comprising the polynucleotide of claim 24.

28. A host cell comprising with the polynucleotide of claim 27.

29. A non-natural trans-cleaving hammerhead ribozyme comprising a core, a stem I, a stem II, a stem II, a bulge within stem I, and a loop II, wherein loop II and the bulge within stem I are derived from loop II and a bulge within stem I of a first hammerhead ribozyme selected from Notophthalmus viridescens satellite RNA (newt), Ambystoma talpoideum Am. ta.), Amphiuma tridactylum (Am. tr.), Schistosoma mansoni hammerhead ribozyme (Schistozyme), D. baccettii cricket hammerhead ribozyme (cricketzyme A), D. schiavazzii cricket hammerhead ribozyme (cricketzyme B), and Avocado sunblotch viroid+ (ASBV+), and wherein at least one of stem II, stem III, and a portion of stem I is derived from a second hammerhead ribozyme that is not the same as the first hammerhead ribozyme.

30. A non-natural trans-cleaving hammerhead ribozyme comprising a core, a stem I, a stem II, a stem III, a bulge within stem I, and a bulge within stem II, wherein the bulge within stem I and the bulge within stem II are derived from a first ribozyme selected from Avocado sunblotch viroid− (ASBV−) and Carnation small viroid-like RNA+ (CarSV+), and wherein at least one of stem III, a portion of stem I, and a portion of stem II is derived from a second hammerhead ribozyme that is not the same as the first hammerhead ribozyme.

31. A non-natural trans-cleaving hammerhead ribozyme comprising strand A of structure I: 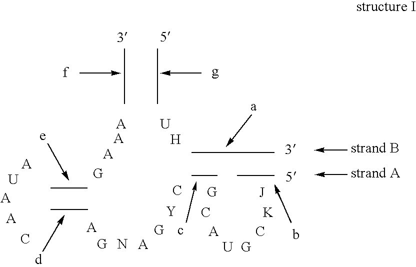
wherein the ribozyme cleaves a target RNA sequence comprising strand B of structure I;
wherein:
J, K, and N are each independently selected from A, C, G, and U;
Y is selected from C and U;
H is selected from A, C, and U;
a is a sequence of 4-20 nucleotides, wherein each nucleotide is independently selected from A, C, G, and U;
b, c, d, e, f, and g are each a sequence of 2-20 nucleotides, wherein each nucleotide is independently selected from A, C, G, and U;
a, b, JKCGUACG, and c together are stem I;
d and e together are stem II; and
f and g together are at least a portion of stem III.

32. A non-natural trans-cleaving hammerhead ribozyme comprising strand A of structure II: 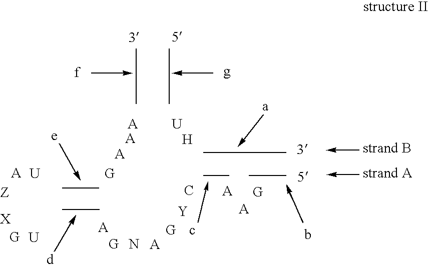
wherein the ribozyme cleaves a target RNA sequence comprising strand B of structure II;
wherein:
N is selected from A, C, G, and U;
Y is selected from C and U;
X is selected from G and C;
Z is selected from G and A;
H is selected from A, C, and U;
a is a sequence of 4-20 nucleotides, wherein each nucleotide is independently selected from A, C, G, and U;
b, c, d, e, f, and g are each a sequence of 2-20 nucleotides, wherein each nucleotide is independently selected from A, C, G, and U;
a, b, GAA, and c together are stem I;
d and e together are stem II; and
f and g together are stem III.

33. The non-natural trans-cleaving hammerhead ribozyme of any of claims 29-32, wherein the ribozyme cleaves the target RNA sequence at an initial rate of at least 0.5 min⁻¹ in a buffer comprising 50 mM Tris (pH 7.0) and 1 mM Mg²⁺ at 37° C.

34. The non-natural trans-cleaving hammerhead ribozyme of any of claims 29-32, wherein the ribozyme cleaves the target RNA sequence at an initial rate of at least 0.5 min⁻¹ in a buffer comprising 50 mM Tris (pH 7.0) and 0.5 mM Mg²⁺ at 37° C.

35. The non-natural trans-cleaving hammerhead ribozyme of any of claims 29-32, wherein the ribozyme cleaves the target RNA sequence at an initial rate of at least 0.5 min⁻¹ in a buffer comprising 50 mM Tris (pH 7.0) and 0.1 mM Mg²⁺ at 37° C.

36. A polynucleotide comprising a nucleic acid encoding the non-natural trans-cleaving hammerhead ribozyme of any of claims 29-32.

37. A vector comprising the polynucleotide of claim 36.

38. A host cell comprising the polynucleotide of claim 36.

39. A pharmaceutical composition comprising the polynucleotide of claim 36 in a pharmaceutically acceptable carrier.

40. A pharmaceutical composition comprising the non-natural trans-cleaving hammerhead ribozyme of any of claims 29-32 in a pharmaceutically acceptable carrier.

41. A method of cleaving a target RNA in a mammal comprising administering the pharmaceutical composition of claim 39, wherein the non-natural hammerhead ribozyme cleaves the target RNA.

42. A method of cleaving a target RNA in a mammal comprising administering the pharmaceutical composition of claim 40, wherein the non-natural hammerhead ribozyme cleaves the target RNA.

43. A method of decreasing the level of a target RNA in a mammal comprising administering the pharmaceutical composition of claim 39, wherein the non-natural hammerhead ribozyme cleaves the target RNA.

44. A method of decreasing the level of a target RNA in a mammal comprising administering the pharmaceutical composition of claim 40, wherein the non-natural hammerhead ribozyme cleaves the target RNA.

45. A method of decreasing the expression of a protein encoded by a target RNA in a mammal comprising administering the pharmaceutical composition of claim 39, wherein the non-natural hammerhead ribozyme cleaves the target RNA.

46. A method of decreasing the expression of a protein encoded by a target RNA in a mammal comprising administering the pharmaceutical composition of claim 40, wherein the non-natural hammerhead ribozyme cleaves the target RNA.

47. A method of cleaving a target RNA sequence in a cell comprising introducing into a cell a polynucleotide comprising a nucleic acid encoding a non-natural trans-cleaving hammerhead ribozyme comprising strand A of structure I: 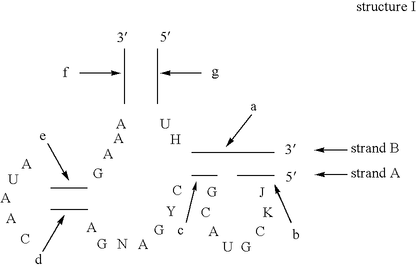
wherein the ribozyme cleaves a target RNA sequence comprising strand B of structure I;
wherein:
J, K, and N are each independently selected from A, C, G, and U;
Y is selected from C and U;
H is selected from A, C, and U;
a is a sequence of 4-20 nucleotides, wherein each nucleotide is independently selected from A, C, G, and U;
b, c, d, e, f, and g are each a sequence of 2-20 nucleotides, wherein each nucleotide is independently selected from A, C, G, and U;
a, b, JKCGUACG, and c together are stem I;
d and e together are stem II; and
f and g together are stem III.

48. A method of cleaving a target RNA sequence in a cell comprising introducing into a cell a polynucleotide comprising a nucleic acid encoding a non-natural trans-cleaving hammerhead ribozyme comprising strand A of structure II: 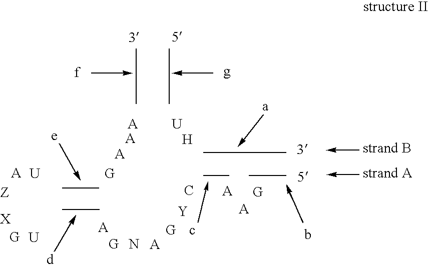
wherein the ribozyme cleaves a target RNA sequence comprising strand B of structure II;
wherein:
N is selected from A, C, G, and U;
Y is selected from C and U;
X is selected from G and C;
Z is selected from G and A;
H is selected from A, C, and U;
a, b, GAA, and c together are stem I;
d and e together are stem II; and
f and g together are stem III.

49. A method of making a trans-cleaving hammerhead ribozyme, comprising:
(a) forming a library of hammerhead ribozyme molecules having structure V: 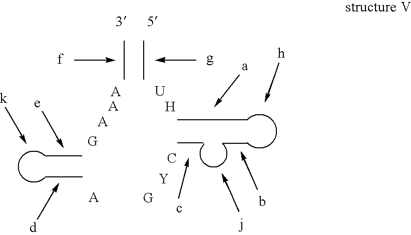
wherein:
N is selected from A, C, G, and U;
Y is selected from C and U;
H is selected from A, C, and U;
a is a sequence of 4-20-nucleotides, wherein each nucleotide is independently selected from A, C, G, and U;
b, c, d, e, f, and g are each a sequence of 2-20 nucleotides, wherein each nucleotide is independently selected from A, C, G, and U;
h is a sequence of 1-20 nucleotides, wherein each nucleotide is independently selected from A, C, G, and U;
j is a bulge of 1-20 nucleotides, wherein each nucleotide is individually selected from A, C, G, and U;
k is a loop of 1-20 nucleotides, wherein each nucleotide is individually selected from A, C, G, and U;
a, b, j, and c together are stem I;
d and e together are stem II;
f and g together are stem III;
wherein each member of the library comprises the same N, Y, H, a, b, c, d, e, f, g, and h; and
wherein each member of the library does not comprise the same j and k.
(b) incubating the library in the presence of less than 1 mM Mg²⁺; and
(c) selecting a hammerhead ribozyme that cleaves at an initial rate of at least 0.5 min⁻¹.
(d) making a trans-cleaving hammerhead ribozyme comprising the nucleic acid sequence b, j, c, CYGANGA, d, k, e, GAAA, f.

50. A method of making a trans-cleaving hammerhead ribozyme, comprising:
(a) forming a library of hammerhead ribozyme molecules having structure V: 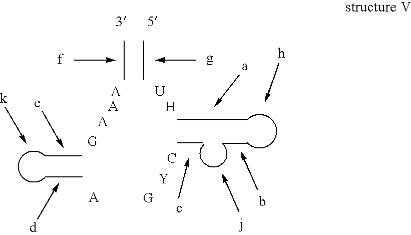
wherein:
N is selected from A, C, G, and U;
Y is selected from C and U;
H is selected from A, C, and U;
a is a sequence of 4-20 nucleotides, wherein each nucleotide is independently selected from A, C, G, and U;
b, c, d, e, f, and g are each a sequence of 2-20 nucleotides, wherein each nucleotide is independently selected from A, C, G, and U;
h is a sequence of 1-20 nucleotides, wherein each nucleotide is independently selected from A, C, G, and U;
j is a bulge of 1-20 nucleotides, wherein each nucleotide is individually selected from A, C, G, and U;
k is a loop of 1-20 nucleotides, wherein each nucleotide is individually selected from A, C, G, and U;
a, b, j, and c together are stem I;
d and e together are stem II;
f and g together are stem III;
wherein each member of the library comprises the same N, Y, b, c, d, e, f, g, h, and j; and
wherein each member of the library does not comprise the same k.
(b) incubating the library in the presence of less than 1 mM Mg²⁺; and
(c) selecting a hammerhead ribozyme that cleaves at an initial rate of at least 0.5 min⁻¹.
(d) making a trans-cleaving hammerhead ribozyme comprising the nucleic acid sequence b, j, c, CYGANGA, d, k, e, GAAA, and f of the selected hammerhead ribozyme.

51. A method of making a trans-cleaving hammerhead ribozyme, comprising:
(a) forming a library of hammerhead ribozyme molecules having structure V: 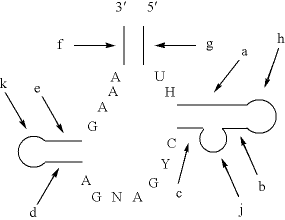
wherein:
N is selected from A, C, G, and U;
Y is selected from C and U;
H is selected from A, C, and U;
a is a sequence of 4-20 nucleotides, wherein each nucleotide is independently selected from A, C, G, and U;
b, c, d, e, f, and g are each a sequence of 2-20 nucleotides, wherein each nucleotide is independently selected from A, C, G, and U;
h is a sequence of 1-20 nucleotides, wherein each nucleotide is independently selected from A, C, G, and U;
j is a bulge of 1-20 nucleotides, wherein each nucleotide is individually selected from A, C, G, and U;
k is a loop of 1-20 nucleotides, wherein each nucleotide is individually selected from A, C, G, and U;
a, b, j, and c together are stem I;
d and e together are stem II;
f and g together are stem III;
wherein each member of the library comprises the same N, Y, H, a, b, c, d, e, f, g, h, and k; and
wherein each member of the library does not comprise the same i.
(b) incubating the library in the presence of less than 1 mM Mg²⁺; and
(c) selecting a hammerhead ribozyme that cleaves at an initial rate of at least 0.5 min⁻¹.
(d) making a trans-cleaving hammerhead ribozyme comprising the nucleic acid sequence b, j, c, CYGANGA, d, k, e, GAAA, and f of the selected hammerhead ribozyme.

52. A method of making a trans-cleaving hammerhead ribozyme, comprising:
(a) forming a library of hammerhead ribozyme molecules having structure VIII: 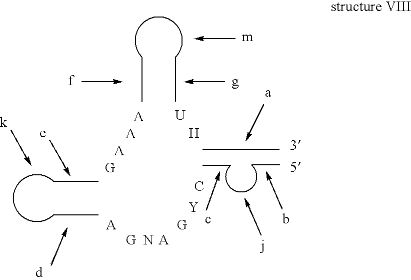
wherein:
N is selected from A, C, G, and U;
Y is selected from C and U;
H is selected from A, C, and U;
a is a sequence of 4-20 nucleotides, wherein each nucleotide is independently selected from A, C, G, and U;
b, c, d, e, f, and g are each a sequence of 2-20 nucleotides, wherein each nucleotide is independently selected from A, C, G, and U;
m is a sequence of 1-20 nucleotides, wherein each nucleotide is independently selected from A, C, G, and U;
j is a bulge of 1-20 nucleotides, wherein each nucleotide is individually selected from A, C, G, and U;
k is a loop of 1-20 nucleotides, wherein each nucleotide is individually selected from A, C, G, and U;
a, b, j, and c together are stem I;
d and e together are stem II;
f and g together are stem III;
wherein each member of the library comprises the same N, Y, H, a, b, c, d, e, f, g, and h; and
wherein each member of the library does not comprise the same j and k.
(b) incubating the library in the presence of less than 1 mM Mg²⁺; and
(c) selecting a hammerhead ribozyme that cleaves at an initial rate of at least 0.5 min⁻¹.
(d) making a trans-cleaving hammerhead ribozyme comprising the nucleic acid sequence b, j, c, CYGANGA, d, k, e, GAAA, f.

53. A method of making a trans-cleaving hammerhead ribozyme, comprising:
(a) forming a library of hammerhead ribozyme molecules having structure VIII: 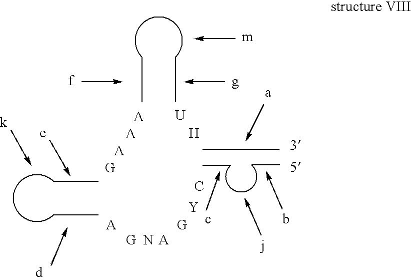
wherein:
N is selected from A, C, G, and U;
Y is selected from C and U;
H is selected from A, C, and U;
a is sequence of 4-20 nucleotides, wherein each nucleotide is independently selected from A, C, G, and U;
b, c, d, e, f, and g are each a sequence of 2-20 nucleotides, wherein each nucleotide is independently selected from A, C, G, and U;
m is a sequence of 1-20 nucleotides, wherein each nucleotide is independently selected from A, C, G, and U;
j is a bulge of 1-20 nucleotides, wherein each nucleotide is individually selected from A, C, G, and U;
k is a loop of 1-20 nucleotides, wherein each nucleotide is individually selected from A, C, G, and U;
a, b, j, and c together are stem I;
d and e together are stem II;
f and g together are stem III;
wherein each member of the library comprises the same N, Y, H, a, b, c, d, e, f, g, h, and j; and
wherein each member of the library does not comprise the same k.
(b) incubating the library in the presence of less than 1 mM Mg²⁺; and
(c) selecting a hammerhead ribozyme that cleaves at an initial rate of at least 0.5 min⁻¹.
(d) making a trans-cleaving hammerhead ribozyme comprising the nucleic acid sequence b, j, c, CYGANGA, d, k, e, GAAA, and f of the selected hammerhead ribozyme.

54. A method of making a trans-cleaving hammerhead ribozyme, comprising:
(a) forming a library of hammerhead ribozyme molecules having structure VIII: 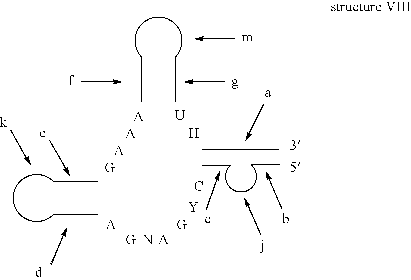
wherein:
N is selected from A, C, G, and U;
Y is selected from C and U;
H is selected from A, C, and U;
a is a sequence of 4-20 nucleotides, wherein each nucleotide is
independently selected from A, C, G, and U;
b, c, d, e, f, and g are each a sequence of 2-20 nucleotides, wherein each nucleotide is independently selected from A, C, G, and U;
m is a sequence of 1-20 nucleotides, wherein each nucleotide is independently selected from A, C, G, and U;
j is a bulge of 1-20 nucleotides, wherein each nucleotide is individually selected from A, C, G, and U;
k is a loop of 1-20 nucleotides, wherein each nucleotide is individually selected from A, C, G, and U;
a, b, j, and c together are stem I;
d and e together are stem II;
f and g together are stem III;
wherein each member of the library comprises the same N, Y, H, a, b, c, d, e, f, g, h, and k; and
wherein each member of the library does not comprise the same
(b) incubating the library in the presence of less than 1 mM Mg²⁺; and
(c) selecting a hammerhead ribozyme that cleaves at an initial rate of at least 0.5 min⁻¹.
(d) making a trans-cleaving hammerhead ribozyme comprising the nucleic acid sequence b, j, c, CYGANGA, d, k, e, GAAA, and f of the selected hammerhead ribozyme.

55. The method of any of claims 49-54, wherein the selecting comprises denaturing polyacrylamide gel electrophoresis (denaturing PAGE).

56. The method of any of claims 49-54, wherein making the trans-cleaving hammerhead ribozyme comprises transcribing a DNA that encodes the sequence b, j, c, CYGANGA, d, k, e, GAAA, f.

57. The method of any of claims 49-54, wherein making the trans-cleaving hammerhead ribozyme comprises chemically synthesizing a nucleic acid comprising the sequence b, j, c, CYGANGA, d, k, e, GAAA, f.
